(19) United States
(12) Patent Application Publication
(10) Pub. No.: US 2025/0009854 A1
(43) Pub. Date: Jan. 9, 2025

(54) Antiviral Agent Comprising a Cellular Entry Receptor and Fc Region Component

(71) Applicant: The Macfarlane Burnet Institute for Medical Research and Public Health Limited, Melbourne, Victoria (AU)

(72) Inventors: Phillip Mark HOGARTH, Williamstown, Victoria (AU); Bruce David WINES, Heidelberg, Victoria (AU)

(73) Assignee: Macfarlane Burnet Institute for Medical Research and Public Health Limited, Melbourne, Victoria (AU)

(21) Appl. No.: 18/708,721

(22) PCT Filed: Oct. 26, 2022
(86) PCT No.: PCT/AU2022/051285

§ 371 Date: May 9, 2024

(30) Foreign Application Priority Data
Nov. 11, 2021 (AU) 2021903608

(51) Int. Cl. A61K 38/48; C12N 9/48

(52) U.S. Cl. CPC A61K 38/4813; C12N 9/485; C12Y 304/17023; C07K 2318/20

(57) ABSTRACT

An immunotherapeutic protein and methods of use and production thereof are disclosed, wherein the immunotherapeutic protein comprises, for example, a cell surface receptor polypeptide which is a cellular entry receptor for the entry of a virus into a host cell, which is linked to a polypeptide comprising an Fc region component. When the cell surface receptor polypeptide is an angiotensin converting enzyme 2 (ACE2) polypeptide or a fragment thereof, the immunotherapeutic protein is capable of binding to a coronavirus spike protein (S protein) and may be useful as an antiviral agent for the prevention or treatment of a coronavirus infection. The Fc region component may comprise an amino acid substitution at the position corresponding to H429 of the amino acid sequence of the human IgG1 heavy chain polypeptide which may enable the production of soluble oligomeric forms or the assembly of oligomeric forms from soluble monomeric forms upon binding to an S protein of a coronavirus (i.e. as present on a virion or the surface of virus-infected cells).

### TECHNICAL FIELD

The present disclosure relates to antiviral agents for treating and/or preventing viral infection, particularly by coronaviruses, using a cell surface receptor polypeptide which is a cellular entry receptor for the entry of a virus into a host cell, linked to a polypeptide comprising an Fc region component.

### PRIORITY DOCUMENT

The present application claims priority from Australian Provisional Patent Application No 2021903608 titled “Antiviral agent” and filed on 11 Nov. 2021, the content of which is hereby incorporated by reference in its entirety.

### BACKGROUND

As of 30 Sep. 2021, it has been reported that COVID-19, caused by the severe acute respiratory syndrome coronavirus 2 (SARS-CoV-2), has caused 233 million infections and 4.7 million deaths globally (COVID-19 Dashboard, by the Center for Systems Science and Engineering (CSSE) at Johns Hopkins University; Dong E et al., Lancet Infect Dis 20(5):533-534, 2020). Publication of the SARS-CoV-2 genome in January 2020 has expedited the development, approval, and deployment of vaccines (Ledford H., Nature Epub 2020. Doi: 10.1038/d41586-020-03593-7; and https://www.gavi.org/vaccineswork/covid-19-vaccine-race). The use of neutralising monoclonal antibodies (mAbs) is another prophylactic and therapeutic response to the COVID-19 challenge, and panels of neutralising mAbs recognising the CoV-2 spike protein (a glycoprotein which facilitates entry of SARS-CoV-2 into host cells) have been isolated from convalescent patients, libraries and humanised mice (see, for example, Liu L et al., Nature 584(7821):450-456, 2020; and Brouwer P J M et al., Science 369(6504):643-650, 2020). Examples of such neutralising mAbs, particularly mAb LY-CoV555 (bamlanivimab, Chen P et al., New Engl J Med 384:229-237, 2020), REGN10933 and REGN10987 (casirivimab and imdevimab when provided in a cocktail) (Hansen J et al., Science 369(6506):1010-1014, 2020, and Baum A et al., Science 369(6506):1014-1018, 2020), and bamlanivimab and etesevimab (mAb LY-CoV016) in combination (Hurt A C and AK Wheatley, Viruses 13(4):628, 2021), have been approved for use in humans.

Thus, the neutralisation of virus, by the targeting of viral entry, is a key aspect of vaccine and therapeutic/preventative (i.e. neutralising) antibody development. However, virus escape mutants comprising variants of the SARS-CoV-2 spike protein (SARS-CoV-2 S) threaten the future efficacy of COVID-19 vaccines and therapeutic antibodies, and underscore a critical vulnerability to pandemic coronaviruses. Notably, SARS-CoV-2 has already demonstrated adaptation to its human host with a D614G mutation first dominating viral strains globally (Korber B et al., Cell 182(4):812-827, 2020) and, more recently, the multiple emergence of strains incorporating an N501Y mutation that has gained dominance in the United Kingdom and South Africa (Zhang g et al., bioRxiv 10.1101/2021.02.02.428884, 2021). Moreover, other strains, particularly showing variation in the receptor binding domain (RBD) of SARS-CoV-2 S and which include examples with little “fitness cost” to the virus (Davis A K F et al., In Ovo J Virol 92:e00859-18, 2018; and Lee J M et al., eLife 8:e49324, 2019), have demonstrated a reduced susceptibility to neutralisation by some individual mAbs and human antisera (Li Q et al., Cell 182(5):1284-1294 e9, 2020). Unfortunately, in combination with other mutations found in the recently emerged variants in South Africa (B.1.351, 20H/501Y.V2) (Tegally H et al., medRxiv 10.1101/2020.12.21.20248640, 2020) and in Brazil (P1, 20J/501Y.V3), the binding of neutralising mAbs (Liu H et al., bioRxiv 10/1101/2021.02.16.431305, 2021; Hastie K M et al., Science 0.1126/science.abh2315, 2021) and convalescent sera (Wibmer C K et al., bioRxiv 10.1101/2021.01.18.427166, 2021) is now impacted. The emergence of these strains, as well as a reality that protective responses are likely to be coronavirus species-specific (NB only a very limited number of mAbs have been isolated which cross-neutralise SARS-CoV-1 and SARS-CoV-2, and antibody responses to SARS-CoV-2 RBD show little or no binding to SARS-CoV-1 or MERS-CoV; Ju B et al., Nature 584(7819):115-119, 2020), means that a critical vulnerability to pandemic coronaviruses is likely to persist unless preventative and/or therapeutic interventions are developed with a more “broad spectrum” effectiveness against coronaviruses.

### SUMMARY

In the study described hereinafter, the inventors have sought to identify and develop novel antiviral agents for treating and/or preventing viral infection in a manner which might also provide a level of cross-neutralisation of multiple virus species or strains (and thereby potentially combat virus escape mutants), by producing agents comprising angiotensin converting enzyme 2 (ACE2), or an appropriate fragment thereof, which constitutes the virus-binding component of the cellular entry receptor for the human pathogenic coronaviruses (e.g.SARS-CoV-1 and SARS-CoV-2) as well as the human endemic coronavirus NL63 (Zhou P et al., Nature 579(7798):270-273, 2020; Hoffmann M et al., Cell 181 (20; 271-280, 2020; and Devarakonda C K V et al., J Immunol doi:10.4049/jimmunol.2001062, 2020), and potential future zoonotic coronaviruses including related coronaviruses of bats (e.g. similar to the bat CoV-RaTG13 virus) and pandolins (e.g. similar to GX-P5L) (Wacharapluesadee S et al., Nat Commun 12(1):972, 2021) in order to provide a “decoy” to block viral interaction and cellular entry in host cells. These agents further comprise an Fc region component to assess whether virus neutralising activity of the ACE2 (or fragment thereof) could be enhanced and/or the beneficial effect of the antiviral agents improved by, for example, inducing or enhancing possible Fc receptor-mediated and complement-based effector functions.

In a first aspect, the present disclosure provides an immunotherapeutic protein comprising an angiotensin converting enzyme 2 (ACE2) polypeptide or a fragment thereof linked to a polypeptide comprising an Fc region component, wherein said immunotherapeutic protein is capable of binding to a coronavirus spike protein (S protein).

As such, the immunotherapeutic protein comprises two polypeptide components; the ACE2 polypeptide (or a fragment thereof) linked to the Fc region component polypeptide thereby forming an “ACE2-Fc” polypeptide as a single fused polypeptide or two covalently linked polypeptides.

The immunotherapeutic protein of the first aspect may comprise a full-length (fl) ACE2 polypeptide or a fragment thereof that is capable of binding to a coronavirus S protein (e.g. CoV-2 S) such as, for example, a fragment of ACE2 comprising all or a portion of the ACE2 ectodomain.

The Fe region component of the immunotherapeutic protein may be derived from any immunoglobulin type (e.g.an IgG or IgA) and may comprise, for example, a full-length (i.e. “complete”) Fc region polypeptide or a minimal fragment of an immunoglobulin heavy (H) chain comprising a CH3 domain (or at least a CH4 domain of an IgE or IgM).

The Fc region component may also be capable of self-associating (e.g. to form an Fc or Fc-like region) such that, in some embodiments, the immunotherapeutic protein is provided in a self-associated (i.e. self-assembled) form between two Fc region components. While comprising two immunotherapeutic proteins of the first aspect, this form is nevertheless referred to herein as a monomer or monomer molecule, and is to be understood as comprising two copies of the ACE2 polypeptide (or a fragment thereof) and two copies of the Fc region component (i.e. an “(ACE2-Fc)₂” protein).

In some embodiments, the immunotherapeutic protein is provided in an oligomeric form such as, for example, a hexamer comprising 6 copies of the monomer described in the preceding paragraph, such that the immunotherapeutic protein comprises twelve copies of the ACE2 polypeptide (or a fragment thereof) and twelve copies of the Fc region component (i.e. an “((ACE2-Fc)₂)₆” protein).

In some other embodiments, the immunotherapeutic protein comprises an ACE2 polypeptide (or a fragment thereof) linked to a polypeptide comprising an Fe region component, wherein said Fe region component comprises an amino acid substitution at the position corresponding to H429 of the amino acid sequence of the human IgG1 heavy chain polypeptide (EU numbering), preferably an H429F or H429Y amino acid substitution.

In a second aspect, the disclosure provides an immunotherapeutic protein comprising a cell surface receptor polypeptide or co-receptor polypeptide or fragment thereof, wherein said cell surface receptor polypeptide is a cellular entry receptor for the entry of a virus into a host cell, linked to a polypeptide comprising an Fc region component, wherein said immunotherapeutic protein is capable of binding to a structural protein of said virus.

The immunotherapeutic protein of the first or second aspect may be useful as an antiviral agent.

In a third aspect, the present disclosure provides an expression construct comprising a polynucleotide sequence encoding the immunotherapeutic protein of the first or second aspect, or a host cell comprising said expression construct for the expression of the immunotherapeutic protein of the first or second aspect.

In a fourth aspect, the present disclosure provides a method for the treatment and/or the prevention of a viral infection in a subject, comprising administering to the subject an effective amount of the immunotherapeutic protein of the first or second aspect.

In a fifth aspect, the present disclosure provides the use of the immunotherapeutic protein of the first or second aspect, for the treatment and/or the prevention of a viral infection in a subject.

In a sixth aspect, the present disclosure provides the use of an immunotherapeutic protein of the first or second aspect, in the manufacture of a medicament for the treatment and/or the prevention of a viral infection in a subject.

In a seventh aspect, the present disclosure provides a pharmaceutical composition or medicament comprising an immunotherapeutic protein of the first or second aspect, and a pharmaceutically acceptable carrier, diluent and/or excipient.

### BRIEF DESCRIPTION OF FIGURES

FIG. 1 provides representations of the human ACE2 polypeptide and ACE2-Fc fusion proteins according to the present disclosure: (A) CryoEM structure of human ACE2 in its membrane context (Yan R et al., Science 367:1444-1448, 2020); (B) flACE2-Fc fusion protein comprising a full-length ACE2 ectodomain fused to a human IgGl Fc region via an S⁷⁴⁰-GGGGS-T²²³ linker sequence (SEQ ID NO: 4), where S⁷⁴⁰ is the last residue of the ACE2 ectodomain and T²²³ is the fusion point to an IgG1 Fc region on the amino-terminus (N-terminus) side of the core hinge sequence containing the inter-heavy chain disulphide bonds; (C) trACE2-Fc fusion protein containing a truncated ACE2 ectodomain fused to an IgG1 Fc region via a D⁶¹⁵-GSGSGSG-T²²³ linker sequence (SEQ ID NO: 6); (D) the truncated ACE2 ectodomain; and (E) provides a schematic representation of the self-association of one embodiment of an ACE2-Fc fusion polypeptide into, first, a monomeric species comprising two copies of the ACE2-Fc fusion polypeptide (i.e. (ACE2-Fc)₂), and then onto a hexameric species, ((ACE2-Fc)₂)₆, through intermediate molecules such as a dimeric species (i.e. ((ACE2-Fc)₂)₂);

FIG. 2 shows the results of the purification of ACE2-Fc fusion proteins according to the present disclosure: (A) Anion exchange (IEX) chromatography of flACE2-Fc-WT (flow through (ft), eluted fractions, and wash) with the ACE2-Fc-containing peak highlighted by *; (B) SDS-PAGE of IEX peak fractions with the flACE2-Fc migrating above the 250 kDa marker and low molecular weight (mw) impurities marked †; (C) Size-exclusion chromatography (SEC) of IEX fractions containing flACE2-Fc-WT using a Superose 6 column, with oligomeric (oli), monomeric (mn) and low mw impurities (†) indicated; and (D) SEC of IEX fractions containing flACE2-Fc-H429Y, showing the high proportion of oligomeric species. The monomeric species are considered to be single molecules (i.e. monomer molecules) comprising two copies of the respective ACE2-Fc fusion polypeptide self-associated through the Fc region components (i.e. (ACE2-Fc)₂);

FIG. 3 provides the results of assays measuring the affinity of ACE2-Fc fusion proteins according to the present disclosure for the SARS-CoV-2 S RBD. The ACE2-Fc fusion proteins were immobilised on anti-human Fc (AHA) biolayer interferometry (BLI) sensors and exposed to RBD. The Dissociation constants, K_{D} (nM), are derived from global fitting of the association and dissociation curves to a Langmuir binding model. BLI sensograms are presented for (A) trACE2-Fc-WT, (B) flACE2-Fc-WT, (C) flACE2-Fc-H429F, (D) EflACE2-Fc-WT (E), flACE2-Fc-WT (F) flACE2-Fc-H429Ymn pH7, (G) flACE2-Fc-H429Ymn pH5, (H) EflACE2-Fc-WT and (I) EflACE2-Fc-H429Ymn. The order of curves in each sensogram correspond, top to bottom, to the order shown in the associated legends: 250 nM-16 nM (A-C), 31 nM-2 nM (D) or 500 nM-16 nM (E-G) or 63 nM-2 nM (H—I);

FIG. 4 provides graphical results showing SARS-CoV-2 RBD-Ig binding activities of ACE2-Fc fusion proteins according to the present disclosure. ACE2-Fc fusion protein binding to immobilised RBD-Ig was determined by ELISA: (A) trACE2-Fc-WT and the following variants thereof:

F: trACE2-Fc proteins including a mutated H429F Fc region
Yₒₗᵢ: SEC purified trACE2-Fc proteins including a mutated H429Y Fc region component (oligomer proteins)
Yₘₙ: SEC purified trACE2-Fc proteins including a mutated H429Y Fc region component (monomer proteins; comprising two copies of the trACE2-Fc fusion polypeptide protein self-associated through the Fc region components)
kif: trACE2-Fc-WT proteins produced in the presence of the mannosidase inhibitor, kifunensine. EC₅₀ (nM) values from the representative curve fits are shown. (B) flACE2-Fc-WT and variants equivalent to those listed above for trACE2-Fc-WT; (C) EflACE2-Fc-WT and variants equivalent to those listed above for trACE2-Fc-WT; and (D) Summary of EC₅₀ binding constants for the trACE2-Fc-WT, flACE2-Fc-WT and variant proteins demonstrating a higher apparent RBD binding affinity for the flACE2-Fc-WT over trACE2-Fc-WT and a weaker binding affinity for the flACE2-Fc-H429Y monomer (Ymn; i.e. comprising two copies of the flACE2-Fc-H429Y fusion polypeptide self-associated through the Fc region components). Welch's unpaired t test, p=0.0332 (*), p<0.0001 (****);

FIG. 5 provides results showing that the ACE2-Fc fusion proteins according to the present disclosure form high molecular complexes with SARS-CoV-2 RBD-Ig and that the flACE2-Fc variants are enzymatically active: (A) The flACE2-Fc-WT and variant proteins and trACE2-Fc-WT were resolved by native polyacrylamide gel electrophoresis (N-PAGE) and showed that the flACE2-Fc fusion protein comprising a mutant H429Y Fc region is composed of oligomeric species, even after separation by SEC into notionally oligomeric (Yₒₗᵢ) and monomeric (Yₘₙ) species; comprising two copies of the fusion polypeptide self-associated through the Fc region components (i.e. (ACE2-Fc)₂ fractions); (B) Gel-shift analysis of ACE2-Fc fusion WT and variant proteins (H429F, F; H429Y, Y) (1 μg, ˜5 μmol) alone (−) or when mixed with SARS-CoV-2 RBD-Ig (0.5 μg, ˜5 μmol; +) and analysed by N-PAGE. The resulting shift in size of the fusion proteins in the mixtures demonstrated the formation of ACE2-Fc: RBD-Ig complexes; and (C) shows the enzymatic activities, as measured by the cleavage of the quenched fluorescence substrate MCA (mean±SEM technical replicates, n as indicated);

FIG. 6 provides results showing that the flACE2-Fc-H429Y fusion protein according to the present disclosure forms pH-dependent oligomers: flACE2-Fc-H429Y purified by IEX was dialysed against (A) PBS 7.4 or (B) 100 mM citrate, 100 mM NaCl pH 5 and then SEC was performed in the same buffers. SEC at pH 5 yielded a greater proportion of monomeric (mn) fusion protein than separation at pH 7.4; t, low mw impurities. (C) Native PAGE (1 μg) of flACE2-Fc-H429Y. Lane 1, SEC pH 5 oligomers (oli); Lane 2, SEC pH 5 monomer (mn); Lane 3, SEC pH 7.4 oligomers; Lane 4, SEC pH 7.4 monomer; Lane 5, SEC pH 5 monomer re-dialysed against PBS pH 7.4. (D) SEC chromatogram of material analysed in lane 2 of panel C, re-dialysed against PBS pH 7.4 and SEC performed in PBS pH 7.4. (E) flACE2-Fc-H429Y monomer (Yₘₙ) prepared at pH 5 is highly active in SARS-CoV-2 RBD-Ig binding activity. The monomers are considered to be single molecules (i.e. monomer molecules) comprising two copies of the respective ACE2-Fc fusion polypeptide self-associated through the Fc region components;

FIG. 7 provides results showing that the SARS-CoV-2 neutralisation potency of ACE2-Fc fusion proteins according to the present disclosure is affected by the ACE2 scaffold (i.e. truncated or full-length) and Fc modification. Neutralisation potencies of the ACE2 polypeptide and the three groups of ACE2-Fc-WT fusion and variant proteins were determined by titration to cytopathic effect (CPE) endpoint in a micro-neutralisation assay. The fusion proteins are trACE2-Fc, flACE2-Fe and EflACE2-Fc WT, and Fc variant, H429F, F; H429Y oligomers on SEC, Yₒₗᵢ; and H429Y monomers on SEC, Yₘₙ(comprising two copies of the fusion polypeptide self-associated through the Fe region components). The trACE2-Fc fusion protein includes the glycan-modified trACE2-Fc-kif. Neutralisation endpoint mean±SEM, unpaired t-tests as indicated, ns=not significant, p<0.0332 (*), p<0.0021 (**), p<0.0002 (***);

FIG. 8 provides graphical results showing that some ACE2-Fc fusion proteins according to the present disclosure exhibit variable binding to Ramos-S cells: (A) ACE2-Fc fusion proteins and variant proteins (5 μg/ml) were reacted with Ramos-S cells and binding determined by flow cytometry, n=3. Binding of concentration series of (B) trACE2-Fc WT and variant proteins, and (C) flACE2-Fc WT and variant proteins, along with EflACE2-Fc-WT were used to determine binding activities to Ramos-S cells by flow cytometry; FI, fluorescence intensity;

FIG. 9 provides the results of assays to assess the interaction of ACE2-Fc fusion proteins according to the present disclosure with FcγR. The ACE2-Fc WT fusion protein and variant proteins (5 μg/ml) were reacted with Ramos-S cells as described in the preceding paragraph. Biotinylated (A) dimeric rsFcγRIIa or (B) dimeric rsFcγRIIIa probes, followed by streptavidin-APC were bound to the cells and detected by flow cytometry. FcγR binding activity of the cells opsonised with ACE2-Fc fusion proteins was readily detected with the exception of ACE2-Fc H429Y fusion proteins which were greatly diminished in FcγR binding activity (3 replicates, mean±SEM); FI, fluorescence intensity;

FIG. 10 provides the results of assays to assess the capacity of ACE2-Fc fusion proteins according to the present disclosure to mediate cell activation via FcγRIIIa. The results demonstrate that flACE2-Fc proteins are potent activators of FcγRIIIa with the exception of the Fc H429Y mutants in any ACE2 format which fail to stimulate. Ramos-S target cells were opsonised with (A) trACE2-Fc, (B) flACE2-Fc and (C) EflACE2-Fc, WT and variant proteins, including H429F, F; H429Y, oligomers, Yₒₗᵢ; H429Y monomer, Yₘₙ (comprising two copies of the fusion polypeptide self-associated through the Fc region components); or trACE2-Fc kif produced from trACE2-Fc WT in Expi293 cells in the presence of the mannosidase inhibitor, kifunensine. In some experiments, Ramos-S target cells were separately opsonised with rituximab, RIT. Opsonised targets were incubated with FcγRIIIa-NF—KB-RE nanoluciferase reporter cells, and FcγRIIIa activation measured by the induction of nanoluciferase activity (relative light units; RLU). Data was fitted to agonist response curves to estimate EC₅₀. (D) EC₅₀ (nM) values from the curve fits are shown. Mean±SEM. n≥4. ANOVA with Dunnett's multiple comparisons test comparing to trACE2-Fc-WT. ns=not significant, p=0.0021 (**), p<0.0001 (****), nd, not determined;

FIG. 11 provides results showing that ACE2-Fc fusion proteins according to the present disclosure comprising Fc regions with the H429F mutation, strongly fix complement and direct complement dependent cytotoxicity of Ramos-S target cells; as determined using ELISA analysis of the complement binding activity of trACE2-Fc (A, C, E) or flACE2-Fc (B, D, F) bound to SARS-CoV-2 RBD-biotin captured by plate bound avidin: C1q binding (A, B) to ACE2-Fc fusion protein variants (2.5 g/ml) at different concentrations of avidin-captured RBD on the plate; C1q binding (C, D) or C5b-9 (E, F) formation by serially diluted ACE2-Fc fusion protein variants bound to avidin-captured RBD-biotin (2.5 μg/ml) (mean±SEM); two independent ELISA experiments. (G) flow cytometric analysis of complement dependent cytotoxicity (CDC) of opsonised Ramos-S cells was determined using a 1:3 dilution of normal human serum as a source of complement (EC₅₀ (nM) values from the curve fits are shown); nf, not fitted;

FIG. 12 provides the results of a multiplex-array assay of competitive inhibitory activity of ACE2-Fc fusion proteins according to the present disclosure to SARS CoV-2 S and its RBD variants and the SARS-CoV-1 (SARS-1) and NL63 spike proteins. The results are presented as “heat maps” showing the capacity of unlabelled trACE2-Fc, flACE2-Fc and EflACE2-Fc fusion proteins, and the comparator human mAb 535, to inhibit the binding of a trACE2-Fc biotin probe to a broad array of CoV-2 RBD variants and the S proteins of SARS-CoV-1 and NL63 (IC₅₀, nM). Biotinylated trACE2-Fc (70 nM) was incubated with a concentration series, eight two-fold dilutions from 282 nM, of unlabelled trACE2-Fc, flACE2-Fc, EflACE2-Fc or the inhibitory human mAb S35 and the binding to RBD or S proteins coupled to beads was determined by multiplex analysis. For inhibition by trACE2-Fc the maximum possible inhibition is 80%. IC50 (nM) values are indicated; NB, no binding;

FIG. 13 provides the results of a flow cytometric analysis of binding (A, C) and CDC (B, D) of purified flACE2-Fc fusion proteins (A, B) and SARS-CoV-2 spike-specific mAbs (C, D) using Ramos-S cells. For the binding analysis, the proteins were serially two-fold titrated and binding activity quantified by flow cytometry. The level of non-specific background binding of fluorescent conjugate to cells is shown (conj only); MFI=Median Fluorescence Intensity. For CDC analysis, the proteins were serially two-fold titrated and diluted human serum used as a source of complement. The CDC lysis was quantitated using Zombie Green. Background lysis by complement in the absence antibodies was determined and is shown (no mAb C′ only); and

FIG. 14 provides graphical results from flow cytometric analysis showing that cooperative and functional synergy of the H429F-modified flACE2-Fc fusion protein and H429F-modified anti-SARS-CoV-2 mAbs further enhances complement dependent killing (CDC). (A) The mAbs S2P6-H429F or S2P6-WT were titrated alone or titrated in the presence of a fixed concentration of the flACE2-Fc-H429 fusion protein (1 μg/ml final concentration) (S2P6-H429F+flACE2-Fc-H429F). When the flACE2-Fc-H429 fusion protein was used alone it mediated CDC killing of 23.4% (indicated by open diamond symbol). Or S2P6-WT was titrated in the presence of a fixed concentration of flACE2-Fc-WT (1 μg/ml final) (S2P6-WT+flACE2-Fc-WT) which, when used alone, mediated a % kill of 5.0% (filled diamond). The background lysis by complement in the absence of mAb or Fe fusion protein is shown (C′ only). Arrow shows examples of greatest synergy; (B) CDC killing potency of H429F-modified flACE2-Fc was evaluated on Ramos-S cells. The mAbs CC40.8—H429F (CC40.8—HF) and CV3-25-H429F mAbs (CV3-25-HF) were used alone (2.5 μg/ml final concentration) or mixed with flACE2-Fc-H429F (CC40.8-HF+flACE2-Fe—HF; CV3-25-HF+flACE2-Fc-HF; note that the final concentrations of the mAbs was 2.5 μg/ml and of the flACE2-Fe—H429F was 1 g/ml). Killing potency was evaluated in the flow cytometric assay using Zombie Green. The % CDC killing mediated by flACE-2-Fc-WT and background lysis by complement in the absence of mAb or Fe fusion protein (C′ only) is also shown. Four replicate values and SEM are shown. Mean value is shown above each column;

FIG. 15 provides representations of immunoglobulin (antibody)-like molecules showing the modular nature of the antibody, and results showing the successful production of such molecules and their function:
(A) Left panel: Depicts an ACE2-Fc Ab-like fusion protein, wherein the ACE2 ectodomain (shown as X1) is present in all of the polypeptide chains; as is described in Example 3 (where an EflACE2 polypeptide is separately linked to both the H chain at the CHi domain and the L chain constant domain, enabling assembly into an H₂L₂ Ab-like fusion protein).
Right panel: Depicts other possible Ab-like fusion proteins comprising fusions to different cell surface receptor polypeptides or co-receptor polypeptides (or fragment thereof) in any combination of specificities (e.g. “X1 X1 X1 X2”, “X1 X1 X2 X2”, “X1 X1 X2 X3 ” or “X1 X2 X3 X4”; where X1, X2, X3 and X4 represent different cell surface receptor polypeptide/co-receptor polypeptide (or fragment thereof);
(B) SDS-PAGE analysis of the EflACE2-Ab-like-WT fusion protein eluted from Protein A affinity matrix with 0.4 M arginine (pH 4) demonstrating that a fully disulphide-linked molecule consistent with an H₂L₂ Ab-like configuration was achieved (lane 1). This molecule comprises an EflACE2 polypeptide fusion to the immunoglobulin constant heavy chain (EflACE2-CH) which self assembles with an equivalent EflACE2 polypeptide fused to the immunoglobulin light chain constant domain (EflACE2-CL). Upon reduction with dithiothreitol, these two chains (i.e. EflACE2-CH (ACE2-CH) and EflACE2-CL (ACE2-CL; lane 2)) are resolved separately.
(C, D) Provide results which show that the EflACE2-Ab-like-H429F fusion protein (H429F) strongly directs CDC of Ramos-S target cells, whereas the corresponding EflACE2-Ab-like-WT fusion protein with H chains of wild type (WT) sequence was ineffective. CDC of the opsonised Ramos-S cells was determined using the presence of a 1/3 dilution of normal human serum as a source of complement. This potent CDC mediated by the EflACE2-Ab-like-H429F fusion protein, was not attributable to different levels of opsonisation compared to the EflACE2-Ab-like fusion protein, as the binding of these to Ramos cells expressing SARS-CoV-2 spike were comparable for both proteins. Binding was determined using flow cytometric analysis using an anti-IgG secondary reagent labelled with goat anti-hIgG Fc FITC. (MFI=median fluorescent intensity; background binding of the anti-Ig fluorescent conjugate shown as (conj only); and

FIG. 16 shows the results of purification of the EflACE2-Ab-like-Fc-H429F fusion protein using Protein A and elution with arginine. (A) Protein A chromatography using Hitrap™ Protein A column with gradient elution from 30 mM arginine (pH 4) to 35% of 130 mM arginine (pH 4); (B) Size-exclusion chromatography (SEC) of the pooled and concentrated Protein A fractions containing EflACE2-Ab-like-Fc-H429F using a Superose 6 Increase 10/300 column with oligomeric material indicated; (HMW) and (C) SDS-PAGE analysis of the pooled Protein A eluate and the pooled SEC monomeric fractions under non-reducing (without DTT, dithiothreitol) and reducing (with DTT) conditions.

### DETAILED DESCRIPTION

The inventors have recognised that the targeting of viral entry provides a potential means for preventing and/or treating viral infection in a manner which might also provide a level of cross-neutralisation of multiple virus species or strains (and thereby potentially combat virus escape mutants), and therefore sought to identify and develop novel antiviral agents targeted to the cellular entry receptor for particular viruses in order to competitively inhibit viruses from infecting their host cells. For the major human pathogenic coronaviruses (i.e. SARS-CoV-1 and SARS-CoV-2), as well as the human endemic coronavirus NL63 and potential future zoonotic coronaviruses including related coronaviruses of, for example, bats and pandolins (Wacharapluesadee et al., 2021 supra), angiotensin converting enzyme 2 (ACE2) is the cell surface receptor for their entry into their host cells (Zhou P et al., Nature 579(7798):270-273, 2020; Hoffmann M et al., Cell 181(20; 271-280, 2020; and Devarakonda C K V et al., J Immunol doi:10.4049/jimmunol.2001062, 2020). ACE2 is a carboxypeptidase expressed on the cell membrane and consists of a cytoplasmic tail, membrane spanning region and an ectodomain. This ectodomain, which is comprised of a catalytic domain (responsible for the carboxypeptidase activity) and a collectrin, or neck, domain thought to be involved in the dimerisation of ACE2 (Yan R et al., 2020 supra), has already been the subject of studies to determine whether it might provide a useful therapeutic and/or preventative antiviral agent by acting as a decoy to block viral interaction and cellular entry in host cells. These studies have used recombinant ACE2 ectodomain in its native form (Zoufaly A et al., Lancet Respir Med 8(11):1154-1158, 2020; and Monteil V et al., Cell 181(4):905-913, 2020), with or without the collectrin domain (Li Y et al., J Virol 94(22):e01283-20, 2020), ACE2 engineered for higher binding affinity to SARS-CoV-2 S (Chan K K et al., Science 369(6508):1261-1265, 2020; and Glasgow A et al., Proc Nat Acad Sci USA 117(45):28046-28055, 2020), ACE2 engineered for higher avidity (Li et al., 2020 supra) or in the form of computational ACE2 decoys (Linsky T W et al., 370(6521):1208-1214, 2020). The inventors have therefore sought to identify and develop novel ACE2-based antiviral agents for the prevention and/or treatment of infections by coronaviruses, such as SARS-CoV-2, and which potentially may be beneficial in the prevention and/or treatment of related, newly emergent virus species and strains.

In a first aspect, the disclosure provides an immunotherapeutic protein comprising an angiotensin converting enzyme 2 (ACE2) polypeptide, or a fragment thereof, linked to a polypeptide comprising an Fc region component, wherein said immunotherapeutic protein is capable of binding to a coronavirus spike protein (S protein) to, for example, inhibit viral infection of a host cell via an ACE2 cell surface receptor of said host cell.

The immunotherapeutic protein of the first aspect may comprise a full-length ACE2 polypeptide, or a fragment thereof, that is capable of binding to a coronavirus S protein (e.g. CoV-2 S) such as, for example, a fragment of ACE2 comprising all or a portion of the ACE2 ectodomain (i.e. the domain of ACE2 that resides outside of the host cell in the membrane-bound ACE2 cellular entry receptor, and which comprises, in the wild type (WT) human protein, amino acids 19 to 740 shown in SEQ ID NO: 1 of Table 2). Suitable portions of the ACE2 ectodomain that may comprise the immunotherapeutic protein include truncated ectodomain fragments comprising at least amino acids 21 to 119 of human ACE2 (which includes residues of ACE2 involved in the interaction of the receptor binding domain (RBD) of CoV-2 S; Wan Y et al., J Virol 94(7):1-9, 2020; and Basit A et al., J Biomol Struct Dyn doi.org/10.1080/07391102.2020.1768150, 2020) or comprising at least amino acids 21 to 450 of human ACE2 (which includes a highly conserved five amino acid sequence (KGDFR: SEQ ID NO: 3) at amino acids 353 to 357 involved in the interaction of the SARS-CoV-2 S RBD; Hayashi T et al., Vet Q 40(1):243-249, 2020) such as a fragment comprising amino acids 19 to 615 (as shown in SEQ ID NO: 2 of Table 2), 21 to 615 or 21 to 740 of human ACE2 or a fragment corresponding to any of those as derived from an ACE2 polypeptide from another species (e.g. a corresponding fragment of an ACE2 polypeptide from primate species such as Pan troglodytes (chimpanzee), Gorilla gorilla gorilla (Western lowland gorilla) and Nomascus leucogenys (Northern white-cheeked gibbon predicted to have very high binding to SARS-CoV-2 spike, or a corresponding fragment of an ACE2 polypeptide from Oryctolagus cuniculus (European rabbit), Saguinus imperator (Emperor tamarin) or Vicugna pacos (Alpaca) predicted to have medium binding to SARS-CoV-2 spike (Damas J et al., Proc Natl Acad Sci USA 117(36):22311-22322, 2020). Other suitable ACE2 fragments may be determined by producing, for example, a series of ACE2 ectodomain truncations (e.g. by recombinant expression) and assaying the fragments for their binding capability to coronavirus S protein using standard methodologies that will be clearly apparent to those skilled in the art. Alternatively, in silico methods such as those described by Basit A et al., 2020 supra may be used.

In some preferred embodiments, the immunotherapeutic protein comprises an ACE2 ectodomain portion comprising the amino acid sequence of amino acids 19 to 740 of SEQ ID NO:1 or an amino acid sequence showing ≥90%, more preferably, ≥95%, and most preferably ≥98% sequence identity to the amino acid sequence of amino acids 19 to 740 of SEQ ID NO:1. As such, the immunotherapeutic protein may comprise, for example, an ACE2 ectodomain portion including one or more natural amino acid variation(s) within the sequence of amino acids 19 to 740 of SEQ ID NO:1 (e.g. as described in, for example, Heinzelman P and PA Romero, bioRxiv 10.1101/2020.09.17.301861, 2020).

In some other preferred embodiments, the immunotherapeutic protein comprises an ACE2 ectodomain portion comprising the amino acid sequence of amino acids 19 to 615 of SEQ ID NO:1 or an amino acid sequence showing ≥90%, more preferably, ≥95%, and most preferably ≥98% sequence identity to the amino acid sequence of amino acids 19 to 615 of SEQ ID NO:1. As such, the immunotherapeutic protein may comprise, for example, an ACE2 ectodomain portion including one or more natural amino acid variation(s) within the sequence of amino acids 19 to 615 of SEQ ID NO:1 (e.g. as described in, for example, Heinzelman and Romero, 2020 supra).

In yet some further preferred embodiments, the immunotherapeutic protein comprises an ACE2 ectodomain portion comprising the amino acid sequence of amino acids 21 to 119 of SEQ ID NO:1 or an amino acid sequence showing ≥90%, more preferably, ≥95%, and most preferably ≥98% sequence identity to the amino acid sequence of amino acids 21 to 119 of SEQ ID NO:1. As such, the immunotherapeutic protein may comprise, for example, an ACE2 ectodomain portion including one or more natural amino acid variation(s) within the sequence of amino acids 21 to 119 of SEQ ID NO:1 (e.g. as described in, for example, Heinzelman and Romero, 2020 supra).

Further, the full-length ACE2 polypeptide, or a fragment thereof, that may comprise the immunotherapeutic protein may be linked either covalently (e.g. “fused”) via a peptide bond, linker sequence (e.g. a short peptide linker sequence) or other chemical linkage (e.g. a disulphide bond (e.g. through one or more cysteine (C) residue) or cross-linker compound) or non-covalently (e.g. by hydrogen bonding) to an exogenous peptide or polypeptide. The exogenous peptide or polypeptide may be linked at the N- or C-terminus of the ACE2 polypeptide (or fragment thereof), but otherwise may be linked at any other suitable site on the ACE2 polypeptide (or fragment thereof). The exogenous peptide or polypeptide may be selected from, for example, immunoglobulin light chain polypeptides, or fragments thereof, such as a constant light (CL) chain domain (e.g. kappa or lambda), peptides or polypeptides of therapeutic significance (e.g. a toxin polypeptide) or one or more other useful activity(ies) or function(s) (e.g. a peptide or polypeptide that may improve protein recovery or expression such as human serum albumin (HSA) and glutathione S-transferase (GST), or provide various affinity-tags such as a polyhistidine tag (His-tag) or a FLAG-tag).

Still further, the full-length ACE2 polypeptide, or a fragment thereof, that may comprise the immunotherapeutic protein may comprise one or more sequence mutation(s). For example, as mentioned above, ACE2 has been engineered for higher binding affinity to SARS-CoV-2 S by introducing a triple mutation (i.e. T27Y, L79T and N330Y) into the ACE2 ectodomain (Chan et al., 2020 supra). Accordingly, the present disclosure extends to immunotherapeutic proteins which comprise a full-length ACE2 polypeptide, or a fragment thereof, (such as those mentioned in paragraph [0038]) comprising, for example, the triple mutation of T27Y, L79T and N330Y. Other such mutations include D30E (Li Y et al., 2020 supra) and K31F, N33D, H34I, H34S and E35Q (Glasgow A et al., Proc Natl Acad Sci USA 117(45):28046-28055, 2020).

The polypeptide comprising an Fc region component (also referred to hereinafter as the “Fe component”) of the immunotherapeutic protein may be derived from one or more immunoglobulin type (e.g. an IgG or IgA) and may comprise a full-length (i.e. “complete”) Fc region polypeptide such as, for example, a heavy chain polypeptide fragment corresponding to that generated by papain digestion (i.e. wherein the polypeptide is cleaved within the upper hinge sequence to generate an Fc region comprising the core hinge sequence (amino acids C226 to C229), the constant heavy domain 2 (CH2; amino acid P230 to K340 of the human IgG1 heavy chain polypeptide (EU numbering)) and the constant heavy domain 3 (CH3; amino acid G341 to G446 or K447 of the human IgG1 heavy chain polypeptide (EU numbering)) and includes similar fragments that may be prepared through digestion of an immunoglobulin heavy chain polypeptide with plasmin and human neutrophil elastase (NHE). Further examples of suitable polypeptides comprising an Fe region component may comprise an entire immunoglobulin heavy chain polypeptide or comprise fragments of the heavy chain polypeptide which comprise, for example, in addition to the CH2 and CH3 domains and the lower and core hinge sequences, all or part of the upper hinge sequence and, optionally, the constant heavy domain 1 (CHI). On the other hand, other suitable Fc region components may comprise fragments of the heavy chain polypeptide which comprise only the CH3 domain (e.g. amino acid G341 to G446 or K447 of the human IgG1 heavy chain polypeptide (EU numbering)), or an equivalent CH4 domain of an IgE or IgM, or a fragment thereof. In addition, suitable polypeptides comprising an Fc region component may comprise a heterogeneous (hybrid) CH3 domain (or CH4 domain) such as a strand exchange engineered domain (SEED) form of a CH3 domain comprising fragments derived from the IgG1 CH3 domain and other proteins such as IgA (Davies A M et al., J Mol Biol 426(3):630-644, 2009).

Further, the polypeptide comprising an Fc region component that may comprise the immunotherapeutic protein may comprise one or more sequence mutations. For example, the Fc component may comprise one or more sequence mutation known to those skilled in the art (see, for example, the examples listed in Table 1 Wang X et al., Protein Cell 9(1); 63-73, 2018; the entire disclosure of which is herein incorporated by reference) such as, for example: mutations that may modulate FcγR binding (e.g. S239D/I332E of IgG1 (EU numbering) which increase FcγRIIIa binding); mutations to improve antibody-dependent cellular cytotoxicity (ADCC) such as S239D/I332E (Lazar G A et al., Proc Natl Acad Sci USA 103(11):4005-4010, 2006), opsonic phagocytosis (e.g. G236A/S239D/I332E; Richards J O et al., Mol Cancer Ther 7:2517-2527, 2008) or complement activation (e.g. S267E/H268F/S324T; Moore G L et al., Mabs 2:181-189, 2010), or decrease effector function (e.g. IgG1: L234A/L235A; IgG4: F234A/L235A; Xu D et al., Cell Immunol 200(1):16-26, 2000); mutations that may increase inhibition via FcγRIIb co-engagement (S267E/L328F; Chu S Y et al., Mol Immunol 45(15):3926-3933, 2008); and mutations which confer enhanced binding to the neonatal Fe receptor (FcRn) such as M252Y/S254T/T256E (Dall'Acqua W F et al., J Immunol 169(9):5171-5180, 2002) to increase in vivo half-life and thereby improved pharmacokinetics (PK).

Other mutations that may be included in the Fc region component include mutations which modulate glycosylation (e.g. a mutation at a position corresponding to Asn297 of the amino acid sequence of the human IgG1 heavy chain polypeptide (EU numbering) such as N297A, N297Q or N297G (Wang X et al., 2018 supra) to provide a site with modified glycosylation (e.g. the lack of glycan at position 297)) to abolish FcγR and complement C1 binding and/or activation. Alternatively, the Fe region component may also be treated to achieve modified glycosylation by producing the immunotherapeutic protein in the presence of kifunensine (a mannosidase inhibitor which prevents normal maturation of the N-linked glycan including core fucosylation of the N-linked glycan); a modification that specifically enhances activity via FcγRIIIa. Further, an Fe component lacking core fucosylation of the Asn297 glycan can also be achieved by culturing host cells expressing the immunotherapeutic protein with inhibitors of fucosylation (e.g. 2-fluoro peracetylated fucose, or similar) or by the expression of enzymes that modify glycosylation pathways (e.g. GDP-6-deoxy-D-lyxo-4-hexulose reductase; Neha M et al., J Biotech 5:100015, 2020) or by modification of these pathways by gene supression (e.g. siRNA silencing of the α-1,6-Fucosyltransferase fucosyl transferase gene FUT8; Imai-Nishiya H et al., BMC Biotechnol 7:84, 2007) or knock-out (Yamane-Ohnuki N et al., Biotechnol Bioeng 87:614-622, 2004).

In some embodiments, the Fc region component is derived from a human immunoglobulin heavy chain polypeptide.

In other embodiments, the Fc region component is derived from an IgG heavy chain polypeptide, and preferably an IgG1 (e.g. human IgG1) heavy chain polypeptide.

The immunotherapeutic protein of the first aspect may be provided in the form of a fusion protein or protein conjugate. Those skilled in the art will understand that in a fusion protein, the ACE2 polypeptide (or fragment thereof) will be covalently linked (i.e. “fused”) to the polypeptide comprising an Fc component (i.e. as a fusion partner) via a peptide bond or linker sequence (e.g. a short peptide linker sequence such as an immunoglobulin hinge sequence on a well-known glycine-serine linker such as GGGGS; SEQ ID NO: 4) at the carboxy-terminus (C-terminus), but more preferably at the N-terminus, of the fusion partner (i.e. Fc component), whereas in a protein conjugate, it is to be understood that the ACE2 polypeptide (or fragment thereof) will be covalently or non-covalently linked to the Fc component (i.e. as a conjugate partner) through a chemical linkage such as a disulphide bond (e.g. through one or more cysteine (C) residue) or cross-linker compound such as a homobifunctional cross-linker such as disuccinimidyl suberate (DSS) (e.g. bis(sulfosuccinimidyl)suberate (BS³); Thermo Fisher Scientific, Waltham, MA, United States of America) or disuccinimidyl tartrate (DST) to link amine groups or a heterobifunctional cross-linker such as m-maleimidobenzoyl-N-hydroxysuccinimide ester (MDS) and N—(ε-maleimidocaproloxy) succinimide ester (EMCS), or by other non-covalent bonding such as hydrogen bonding. Where the Fe component is a conjugate partner, the immunotherapeutic protein conjugate may be considered as a cross-linked protein, and the Fc component may be conjugated to the ACE2 polypeptide (or fragment thereof) at the N-terminus, but more preferably at the C-terminus, but otherwise at any other suitable site on the ACE2 polypeptide (or fragment thereof) such as, for example, at a site of glycosylation. Alternatively, the Fc component (as a conjugate partner) may be conjugated to the ACE2 polypeptide (or fragment thereof) at the N- or C-terminus of the Fc component or otherwise at any other suitable site on the Fc component (e.g. within CH1 or the hinge sequence if these are included in the polypeptide comprising an Fc region component).

Preferably, the immunotherapeutic protein of the first aspect is provided in the form of a fusion protein comprising the ACE2 polypeptide (or fragment thereof) fused to the N-terminus of the Fc component via a peptide bond or short peptide linker sequence.

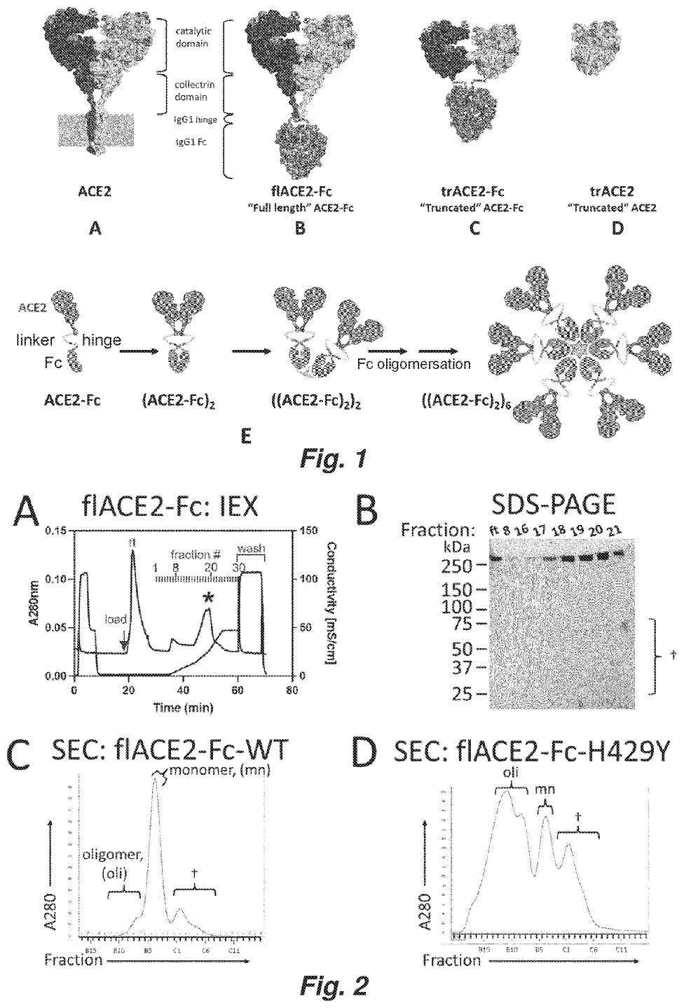

The Fc region component may be capable of self-associating (e.g. to form an Fc or Fc-like region) such that, in some embodiments, the immunotherapeutic protein of the first aspect is provided in a self-associated (i.e. self-assembled) form. For example, in some embodiments, the immunotherapeutic protein comprises a single polypeptide chain, wherein the polypeptide chain comprises one copy of the ACE2 polypeptide (or a fragment thereof) fused to the Fc region component, while in other embodiments, the immunotherapeutic protein comprises, for example, two of such polypeptide chains and wherein the Fc region components self-associate by non-covalent bonding such as hydrogen bonding or through the formation of disulphide bonds through one or more cysteine (C) residue, particularly where situated within the hinge sequence, especially the core hinge sequence (if present) (Yoo E M et al., J Immunol 170:3134-3138, 2003). In such a monomer molecule, the immunotherapeutic protein comprises two Fc region component polypeptides (associated to one another) and two fused/conjugated ACE2 polypeptides (or two fragments thereof), and as such may be considered as being bivalent in respect of the ACE2 polypeptide (or fragment thereof) (see FIG. 1) and likely to show enhanced antiviral potency by increased avidity of binding to coronavirus spike protein as well as possible increased virus neutralisation activity (e.g. conferred by cross-linking spike protein and/or aggregating viral particles (virions)).

Thus, in some embodiments, the Fc region component polypeptide comprises a core hinge sequence to enable self-association through the formation of inter-chain disulphide bonds between one or more cysteine (C) residues of the core hinge sequence of two Fc region components. It is to be noted that in the Examples and Figures hereinafter, such self-associated forms of the immunotherapeutic protein are considered to be single protein molecules (i.e. each comprising two copies of the fusion polypeptide or conjugated polypeptide self-associated through the Fc region components) and are referred to as monomers/monomeric.

In yet other embodiments, the immunotherapeutic protein comprises two polypeptides that are non-identical (i.e. heterodimeric), wherein the Fe region components self-associate (i.e. to form the single protein molecule comprised of two different polypeptide chains) either by non-covalent bonding such as hydrogen bonding or through the formation of disulphide bonds across the core hinge sequences (if present), but wherein only one of the Fc region component polypeptides is fused/conjugated to an ACE2 polypeptide (or fragment thereof).

The immunotherapeutic protein of the first aspect may also be oligomeric. For instance, the immunotherapeutic protein region may form soluble oligomers at neutral pH (such as physiological pH of about 7.4), or the immunotherapeutic protein may form oligomers upon binding to the target, that is a spike protein of a coronavirus present on a viral particle (virion) or surface of virus-infected cells (i.e. through “on target” oligomerisation).

For example, certain mutations of the Fc region have been identified which enable, at physiological pH, self-association of the mutated Fc regions into soluble oligomeric forms (e.g. forms comprising 3 copies, 4 copies, 5 copies, or 6 copies of the monomer (e.g. a hexamer assembled from 6 monomers each comprising two fusion protein chains, such that this oligomeric form of the immunotherapeutic protein comprises, in total, 12 copies of the fusion or conjugate protein comprising an ACE2 polypeptide (or fragment thereof) linked to a polypeptide comprising an Fc region component). These mutations include an amino acid substitution at the position corresponding to H429 of the amino acid sequence of the human IgG1 heavy chain polypeptide (EU numbering). Included among the suitable mutations at this site are: H429X¹, where X¹ is selected from tyrosine (i.e. H429Y), methionine, isoleucine, leucine, tryptophan and valine;

but preferably, the immunotherapeutic protein comprises an H429Y mutation.

Where immunotherapeutic protein is oligomeric, it is also to be understood that the immunotherapeutic protein may be hetero-oligomeric (i.e. wherein the oligomer is formed from two or more different polypeptides).

Other Fc region mutations which enable self-association of monomers and/or dimers of the immunotherapeutic proteins into oligomeric forms (e.g. hexameric forms), at physiological pH, may be readily determined by producing, for example, a series of mutated Fc regions (e.g. comprising one or more amino acid substitution) by, for example, recombinant expression, and determining the molecular weight of the expressed proteins (e.g. by mass spectrometry, standard size-based chromatography techniques and non-denaturing electrophoretic assays (e.g. native-PAGE) as is well known to those skilled in the art.

In some particular embodiments, the immunotherapeutic protein of the first aspect is soluble and oligomeric at physiological pH, and comprises a polypeptide comprising an Fc region component which includes a single mutation enabling self-association of monomers of the immunotherapeutic proteins into oligomeric forms, wherein said single mutation is an amino acid substitution at the position corresponding to H429 of the amino acid sequence of the human IgG1 heavy chain polypeptide (EU numbering). For the avoidance of doubt, where the Fc region component is derived from another immunoglobulin type or isotype (or from an immunoglobulin from another species), the position corresponding to H429 of the human IgG1 heavy chain polypeptide IgG1 includes the examples shown in Table 1.

TABLE 1

| Immunoglobulin | H429 |
|---|---|
| Human IgG2 | H429 |
| Human IgG3 | H429 |
| Human IgG4 | H429 |
| Human IgA1 and IgA2 (numbered by | H436 |
| correspondence to Bur IgA1 myeloma) | |
| Chimpanzee/Gorilla/Macaque/Orangutan | H429 |
| IgG1 | |

Alternatively, oligomeric forms of the immunotherapeutic protein may be produced by employing other techniques well known to those skilled in the art such as, for example, Fc multimeric forms (Stradomers™) comprising linked multimerisation domain (MD) sequences from the hinge region of human IgG2 or the isoleucine zipper (ILZ) to the N- or C-terminus of IgG, e.g. murine IgG2a (Fitzpatrick E A et al., Front Immunol 11, article 496, 2020), sequences from IgM (Mekhalel D N et al., Sci Rep 1:124, 2011), or dimerisation sequences from unrelated proteins such as cyclic adenosine monophosphate-dependent protein kinase and A-kinase anchoring proteins (Rossi E A et al., Blood 113(24):6161-6171, 2009).

Preferably, monomeric or oligomeric forms of the immunotherapeutic protein are soluble (i.e. in physiological saline).

In some particular embodiments, the immunotherapeutic protein of the first aspect comprises a hexamer (at physiological pH) of monomer forms of the immunotherapeutic protein, each comprised of two fusion polypeptide chains or two conjugate proteins; that is, a hexamer comprising 12 copies of the ACE2 polypeptide (or a fragment thereof) and 12 copies of the Fc region component.

While not wishing to be bound by theory, it is considered that where the immunotherapeutic protein is capable of forming oligomers at physiological pH through self-association, the immunotherapeutic protein may be particularly suited for achieving virus neutralisation due to enhanced avidity for spike proteins of coronavirus, and without perhaps optimal involvement of complement activation (since it was found in the study described hereinafter, that an immunotherapeutic protein with an Fe component comprising an H429Y mutation showed complement fragment, C1q and C5b-C9, fixing activity like that of a wild type (WT) Fc component in the context of a trACE2-Fc-H429Y fusion protein, but abrogated complement activation in the context of a flACE2-Fc-H429Y fusion protein). Similarly, complement-dependent cytotoxicity (CDC) using trACE2-Fc-H429Y fusion protein showed only modestly more potent lytic activity than trACE2-Fc-WT and thereby may be particularly suited for achieving virus neutralisation due to enhanced avidity for spike proteins of coronavirus, and without perhaps optimal involvement of complement activation of, for example, cells infected with coronavirus or undergoing infection with coronavirus. As such, in some embodiments, wherein the immunotherapeutic protein comprises an ACE2 polypeptide (or a fragment thereof) linked to a polypeptide comprising an Fc region component including an H→Y amino acid substitution at the position corresponding to H429 of the amino acid sequence of the human IgG1 heavy chain polypeptide (EU numbering), the immunotherapeutic protein may show enhanced antiviral activity (e.g. anti-SARS-CoV-2 activity) by neutralisation of infectivity by inhibiting viral entry.

On the other hand, and again while not wishing to be bound by theory, it is considered that where the immunotherapeutic protein may form oligomers (or dimers) upon binding to a spike protein of a coronavirus (i.e. present on a virion or the surface of virus-infected cells), the immunotherapeutic protein may be less suited for achieving virus neutralisation, but may show an enhanced capability for complement activation and thereby complement-dependent cytotoxicity (CDC) of, for example, virions or cells infected with coronavirus or undergoing infection with coronavirus. In some embodiments of such an immunotherapeutic protein, the immunotherapeutic protein may comprise a polypeptide comprising an Fc region component which comprises an amino acid mutation at a position corresponding to H429 and, optionally, K447 of the amino acid sequence of the human IgG1 heavy chain polypeptide (EU numbering). Included among the suitable mutations at these sites are:
H429X², where X² is selected from phenylalanine (i.e. H429F), glutamate, glutamine and serine; and
K447X³, where X³ is selected from null (i.e. K447del; an amino acid deletion or truncation of the Fc component), and glutamate (i.e. K447E) (see van der Bremer E T J et al., mAbs 7(4):672-680, 2015);
   but preferably, the immunotherapeutic protein includes the following mutations: H429F and, optionally, K447del (e.g. an immunotherapeutic protein comprising an H429F mutation). In addition, the Fe region component may comprise one or more sequence mutations known to those skilled in the art (such as those described above at paragraph [0046]). The ACE2 polypeptide (or fragment thereof) of an immunotherapeutic protein of these embodiments may preferably comprise a portion of the ACE2 ectodomain which excludes a collectrin domain.

In any case, in some embodiments, the immunotherapeutic protein of the first aspect comprises an ACE2 polypeptide (or a fragment thereof) linked to a polypeptide comprising an Fc region component, wherein said Fc region component comprises an amino acid substitution at the position corresponding to H429 of the amino acid sequence of the human IgG1 heavy chain polypeptide (EU numbering). Such an immunotherapeutic protein may be monomeric or oligomeric. Preferably, the monomeric or oligomeric forms are soluble (i.e. in physiological saline). Monomeric forms may form oligomers (or dimers) upon binding to a spike protein of a coronavirus (i.e. present on a virion or the surface of virus-infected cells). The amino acid substitution at the position corresponding to H429 may be the only mutation in the Fc component. Preferably, the amino acid substitution at the position corresponding to H429 is H429F. As such, in some embodiments, wherein the immunotherapeutic protein comprises an ACE2 polypeptide (or a fragment thereof) linked to a polypeptide comprising an Fc region component including an H→F amino acid substitution at the position corresponding to H429 of the amino acid sequence of the human IgG1 heavy chain polypeptide (EU numbering), the immunotherapeutic protein may show enhanced antiviral activity (e.g. anti-SARS-CoV-2 activity) by enhanced complement activation and complement-dependent cytotoxicity (CDC) of, for example, cells infected with coronavirus or undergoing infection with coronavirus, or even inactivation/clearance (“killing”) of the virus itself (e.g. virions).

In some embodiments, the immunotherapeutic protein of the first aspect is an antibody-like molecule (Ab-like molecule). Such molecules are not antibodies, but minimally comprise an ACE2 polypeptide or a fragment thereof linked to a polypeptide comprising an Fe region component (e.g. to at least a CH3 domain (or a CH4 domain) of an immunoglobulin heavy (H) chain (providing the molecule with an “H-like” chain). Examples of such molecules are depicted in FIG. 15A. As can be seen, the molecules may have the standard H₂L₂ antibody structure, wherein each H-like chain is self-assembled with an “L-like” chain (e.g. comprising an ACE2 polypeptide or fragment thereof linked to an immunoglobulin light (L) chain constant domain) or an H2 format (where the H-like chains are produced in the absence of L chains).

The immunotherapeutic protein may be useful as an antiviral agent. Such an antiviral agent may be useful in the treatment of a coronavirus infection (e.g. a SARS-CoV-2 infection) in a subject and/or the prevention of a coronavirus infection (e.g. a SARS-CoV-2 infection) in a subject. That is, the immunotherapeutic protein may be useful as a therapeutic and/or preventative antiviral agent. As indicated above, and while not wishing to be bound by theory, the immunotherapeutic agent may act to achieve virus neutralisation by acting as a decoy to block viral interaction and cellular entry into host cells.

In use as an antiviral agent, the immunotherapeutic protein of the first aspect may provide one or more beneficial activity(ies) or characteristic(s) over an antiviral agent that may comprise an ACE2 polypeptide (or fragment thereof) alone (i.e. without an Fc region component). For example, it may be anticipated that the Fc region component may provide improved pharmacokinetic properties (e.g. a longer plasma half-life) by one or more mechanism such as improving stability, providing resistance to proteases and/or through interaction with the neonatal Fc receptor (FcRn) which is known to be involved in the binding and/or transportation of proteins such as IgG and albumin to thereby extend the plasma half-life of such proteins (Stapleton N M et al., Immunol Rev 268(1):253-268, 2015). In addition, by the inclusion of the Fc region component, the immunotherapeutic protein may attract, for example, Fc receptor-mediated and complement-based effector functions.

Thus, in some embodiments, the immunotherapeutic protein may attract an Fc-mediated effector function selected from antibody-dependent cell-mediated cytotoxicity (ADCC), antibody-dependent cell-mediated phagocytosis (ADCP) or endocytosis, removal of immune complexes from the circulatory or lymphatic systems, and/or Fe-mediated cytokine release (Chenoweth A M et al., Immunol Cell Biol 98(4):287-304, 2020). In the study described hereinafter, it was found that the use of an immunotherapeutic protein of the first aspect that comprises a full-length ACE2 ectodomain fragment including the collectrin domain may show enhanced binding and/or activation of FcγR and may therefore be preferred where an Fe receptor-mediated effector function is desired. In addition, it was found that the inclusion of an Fc component comprising modified glycosylation may also enhance FcγR activation.

And in other embodiments, the immunotherapeutic protein, through interaction with the inhibitory FcγRIIb, may inhibit, for example, the antibody-dependent inflammation that has been observed with some coronaviruses (e.g. by inhibiting the activation of effector cells expressing FcγRIIb that may in turn thwart the production of proinflammatory cytokines and mediators: Jaume M et al., J Virol 85(20):10582-10597, 2011; and Wen J et al., Int J Infect Dis 100:483-489, 2020), and/or enable “sweeping” and removal of coronavirus complexed with the immunotherapeutic protein in a manner analogous to the removal or sweeping of immune complexes including viral particles (virions) from the circulatory or lymphatic systems (Chenoweth et al., 2020 supra).

Further, in still other embodiments, the immunotherapeutic protein may induce a complement-based effector function through the Fc component initiating the classical pathway of the complement cascade by binding of C1q to lead to the activation of other plasma complement proteins and culminating in lysis by complement-dependent cytotoxicity (CDC) of, for example, cells infected with coronavirus or undergoing infection with coronavirus, or even inactivation/clearance (“killing”) of coronavirus itself (e.g. virions). In the study described hereinafter, it was found that the use of an immunotherapeutic protein of the first aspect that comprises a truncated ACE2 polypeptide, that is a fragment of the ACE2 polypeptide comprising a portion of the ectodomain which excludes a collectrin domain (e.g. a fragment comprising amino acids 19 to 615 of human ACE2) may be particularly suitable for initiating CDC if desired.

In some embodiments, it may be desirable to avoid, for example, any Fc receptor-mediated effector function, in which case it may be preferred that the immunotherapeutic protein comprise a polypeptide comprising an Fc region component which comprises certain amino acid substitutions at the position corresponding to H429 of the amino acid sequence of the human IgG1 heavy chain polypeptide (EU numbering), since in the study described hereinafter it was found that such an immunotherapeutic protein with an Fc component comprising an H429Y mutation had diminished activity to bind and/or ability to activate FcγR.

It is considered that the immunotherapeutic protein may be readily modified for use as an antiviral agent for the prevention and/or treatment of an infection by a virus other than a coronavirus by replacing the ACE2 polypeptide with another relevant cell surface receptor or fragment thereof, or co-receptor, for the entry of the other virus into a host cell.

Thus, in a second aspect, the disclosure provides an immunotherapeutic protein comprising a cell surface receptor polypeptide, co-receptor polypeptide or fragment thereof, wherein said cell surface receptor polypeptide is a cellular entry receptor for the entry of a virus into a host cell, linked to a polypeptide comprising an Fc region component, wherein said immunotherapeutic protein is capable of binding to a structural protein of said virus to, for example, inhibit viral infection of a host cell via the cell surface receptor of said host cell.

The immunotherapeutic protein of the second aspect may comprise a full-length cell surface receptor polypeptide or a fragment thereof that is capable of binding to a structural protein of said virus such as, for example, a fragment comprising all or a portion of an ectodomain of the cell surface receptor (i.e. the domain of the cell surface receptor that resides outside of the host cell in the membrane-bound receptor). Alternatively, the immunotherapeutic protein of the second aspect may comprise a full-length co-receptor polypeptide (i.e. a co-receptor of the cell surface receptor polypeptide) or a fragment thereof that is capable of binding to a structural protein of said virus such as, for example, a fragment comprising all or a portion of an ectodomain of the co-receptor (i.e. the domain of the co-receptor that resides outside of the host cell in the membrane-bound receptor).

Examples of cell surface receptor polypeptides (or fragments thereof) that may comprise the immunotherapeutic protein of the second aspect include Hsp70 (the cellular entry receptor for Japanese encephalitis virus), hepatitis A virus cellular receptor 1 (HAVCR1/TIM-1; the cellular entry receptor for hepatitis A virus and ebola virus), cluster of differentiation 155 (CD155; the cellular entry receptor for poliovirus), intracellular adhesion molecule 1 (ICAM-1, aka CD54; a cellular entry receptor for human rhinoviruses; Insulin-like growth factor-1 receptor (IGF1R; a cellular entry receptor for human respiratory syncytial virus (RSV)); glucose transporter 1 (GLUTI; the cellular entry receptor for human T cell leukemia virus 1), the CD4 receptor (the cellular entry receptor for human immunodeficiency virus (HIV)), and dipeptidyl peptidase-4 (DPP4, aka CD26; the entry receptor for MERS, Middle East respiratory syndrome coronavirus (MERS-CoV), to name just a few.

Examples of co-receptor polypeptides (or fragments thereof) that may comprise the immunotherapeutic protein of the second aspect include C—X—C chemokine receptor type 4 (CXCR4), C—C chemokine receptor type 5 (CCR5)(co-receptors of the CD4 receptor which binds to the HIV viral glycoprotein gp120 and enables HIV to fuse with the host cell membrane), nucleolin (which is a co-receptor involved in the cellular entry of RSV) and occludin (which are co-receptors required to enable infection by hepatitis C virus (HCV)), among many others.

The Fc region component of the immunotherapeutic protein of the second aspect may be as described above at, for example, paragraphs [0044] to [0048], [0054], [0055] and [0062].

The immunotherapeutic protein of the second aspect may be provided in the form of a fusion protein or protein conjugate. Preferably, the immunotherapeutic protein of the second aspect is provided in the form of a fusion protein comprising the cell surface receptor polypeptide (or fragment thereof) fused to the N-terminus of the Fc component via a peptide bond or linker sequence (e.g. a short peptide linker sequence such as an immunoglobulin hinge sequence or a glycine-serine linker).

The immunotherapeutic protein of the second aspect may be monomeric, comprising two copies of, for example, a fusion protein or protein conjugate comprising a cell surface receptor polypeptide or co-receptor polypeptide (or fragment thereof) linked to a polypeptide comprising an Fc region component, but, may also be oligomeric at physiological pH (e.g. a hexameric form comprising, in total, 12 copies of the cell surface receptor polypeptide or co-receptor polypeptide (or fragment thereof) and 12 copies of the Fc region component), wherein the Fe region component comprises one or more mutation (e.g. as described above in, for example, paragraph [0055]) which enables self-association of the immunotherapeutic proteins into oligomeric forms.

In some other embodiments, the immunotherapeutic protein of the second aspect comprises a cell surface receptor polypeptide or co-receptor polypeptide (or fragment thereof) linked to a polypeptide comprising an Fe region component, wherein said Fc region component comprises an amino acid substitution at the position corresponding to H429 of the amino acid sequence of the human IgG1 heavy chain polypeptide (EU numbering). Such an immunotherapeutic protein may be monomeric or oligomeric (including hetero-oligomeric). Preferably, monomeric or oligomeric forms are soluble (i.e. in physiological saline). The monomeric forms may form oligomers upon binding (i.e. to a structural protein of the virus). The amino acid substitution at the position corresponding to H429 may be the only mutation in the Fc component. For example, in some particular embodiments, the amino acid substitution at the position corresponding to H429 is H429Y or H429F.

In yet some other embodiments, the immunotherapeutic protein of the second aspect comprises a cell surface receptor polypeptide or co-receptor polypeptide (or fragment thereof) linked to a polypeptide comprising an Fc region component, wherein said Fc region component includes an amino acid mutation at the position corresponding to H429 and, optionally, K447 of the amino acid sequence of the human IgG1 heavy chain polypeptide (EU numbering). Included among the suitable mutations at the K447 site is K447del.

Oligomeric forms of the immunotherapeutic protein of the second aspect may be produced by employing other techniques such as those mentioned above such as, for example, the use of Fc multimeric forms (Stradomers™) comprising linked multimerisation domain (MD) sequences from the hinge region of human IgG2 or the isoleucine zipper (ILZ) to the N- or C-terminus of murine IgG2a; Fitzpatrick et al., 2020 supra).

In some embodiments, the immunotherapeutic protein of the second aspect is an antibody-like molecule (Ab-like molecule) such as is discussed above in respect of the immunotherapeutic proteins of the first aspect. However, in this case, the molecules minimally comprise one or more cell surface receptor polypeptide or co-receptor polypeptide or fragment thereof that or which is other than an ACE2 polypeptide linked to a polypeptide comprising an Fe region component (e.g. to at least a CH3 domain (or a CH4 domain) of an immunoglobulin heavy (H) chain (to provide the molecule with an “H-like” chain), and may, for example, have a H₂ format or a format like the standard H₂L₂ antibody structure, wherein each H-like chain is self-assembled with an “L-like” chain (e.g. comprising cell surface receptor polypeptide or co-receptor polypeptide or fragment thereof linked to an immunoglobulin light (L) chain constant domain). As depicted in FIG. 15A, for an Ab-like protein with a H₂L₂ format, the protein may comprise a total of four different cell surface receptor polypeptide/co-receptor polypeptide (or fragment thereof) (i.e. wherein each of X1, X2, X3 and X4 in the depicted molecule are different). The different cell surface receptor polypeptide/co-receptor polypeptide (or fragment thereof) may be targeted against the same virus or against different viruses or viral stains (e.g. to provide an Ab-like protein for use as tetravalent antiviral agent).

Immunotherapeutic proteins according to the present disclosure may be produced in accordance with any of the standard methodologies known to those skilled in the art. For instance, those skilled in the art can readily prepare an immunotherapeutic fusion protein by generating a construct, using standard molecular biology techniques, which comprises a polynucleotide sequence encoding the fusion protein, introducing the construct into a suitable host cell (e.g. a human kidney (HEK) host cell or derivative thereof such as Expi293 cells (Thermo Fisher Scientific)) for expression of the fusion protein, culturing the host cells according to standard culturing protocols and recovering the expressed fusion protein from the culture supernatant using, for example, any of the known suitable methodologies for purification (e.g. affinity chromatography (e.g. Protein A), ion exchange chromatography (IEX), size exclusion chromatography (SEC) and combinations thereof). In the study described hereinafter, it was found that where it is desirable that the immunotherapeutic protein be in a monomeric form, the recovery of the expressed protein be preferably conducted under conditions of mildly acidic pH (e.g. a pH of less than neutral pH such as pH 5.0), while if an oligomeric form of the immunotherapeutic protein is desired, then preferably the recovery of the expressed protein will be conducted under conditions of substantially neutral pH (e.g. a pH in the range of 7.0 to 8.5, preferably 7.5 to 8.0). Further, it was found that where affinity chromatography (e.g. Protein A) is used in the purification, especially where the immunotherapeutic proteins are in the form of an Ab-like molecule, the use of mild elution conditions such as the use of an elution buffer comprising a low concentration of arginine (e.g. less than 130 mM) and at less than or equal to pH 5, is advantageous so as to suppress the formation of aggregates. This can be achieved by, for example, standard liquid chromatography systems that can deliver a buffer gradient to the column, in this case, a linear gradient to, for example, 35% of 130 mM arginine (pH 4.0).

Thus, in some preferred embodiments, the recovery of an expressed immunotherapeutic protein according to the present disclosure is:
(i) preferably conducted under conditions of mildly acidic pH (e.g. a pH of less than neutral pH such as pH 6.5, preferably pH 5.5, and more preferably, pH 5.0) where it is desired that the immunotherapeutic protein be provided in a monomeric form; or
(ii) where it is desired that the immunotherapeutic protein be provided in an oligomeric form (e.g. as a hexamer), preferably conducted under conditions of substantially neutral pH (e.g. a pH in the range of 7.0 to 8.5, preferably 7.5 to 8.0, or more preferably, at physiological pH of about 7.4); or
(iii) preferably conducted using a method comprising affinity chromatography using an elution buffer comprising a low concentration of arginine (e.g. less than 130 mM) and at less than or equal to pH 5.0 (preferably about pH 4.0), especially where it is desired that the immunotherapeutic protein be provided as an antibody-like molecule.

Further, in some particular embodiments, the recovery of an expressed immunotherapeutic protein according to the present disclosure comprises recovery by size exclusion chromatography (SEC), for example under conditions of mildly acidic pH for the production of monomeric forms of the immunotherapeutic protein, or under substantially neutral pH for the production of the immunotherapeutic protein in oligomeric forms. The SEC may, if desired, follow a recovery stage comprising ion exchange chromatography (IEX).

In a third aspect, the present disclosure provides an expression construct comprising a polynucleotide sequence encoding the immunotherapeutic protein of the first or second aspect, or a host cell comprising said expression construct for the expression of the immunotherapeutic protein of the first or second aspect.

In a fourth aspect, the present disclosure provides a method for the treatment and/or the prevention of a viral infection in a subject, comprising administering to the subject an effective amount of the immunotherapeutic protein of the first or second aspect.

The method of the present disclosure will be typically applied to the treatment and/or prevention of a viral infection in a human subject. However, the subject may also be selected from, for example, livestock animals (e.g. cows, horses, pigs, sheep and goats), companion animals (e.g. dogs and cats) and exotic animals (e.g. non-human primates, tigers, elephants etc).

In some embodiments, the immunotherapeutic protein may be administered with an antibody directed against the target virus. Where the immunotherapeutic protein comprises an ACE2 polypeptide (or a fragment thereof) which binds with the RBD of the CoV-2 spike protein, the antibody may be selected from antibodies that are, for example, broadly neutralising coronavirus mAbs (ie bNmAbs which can neutralise multiple coronavirus types or strains), broadly reactive coronavirus mAbs (ie mAbs which may not be neutralising but can bind with multiple coronavirus types or strains), broadly neutralising SARS-CoV-2 mAbs (ie bNmAbs which can neutralise multiple SARS-CoV-2 strains) and broadly reactive SARS-CoV-2 mAbs, broadly neutralising coronavirus spike stem specific mAbs, broadly reactive coronavirus spike stem specific mAbs, broadly neutralising SARS-CoV-2 spike stem specific mAbs, and broadly reactive SARS-CoV-2 spike stem specific mAbs. In some specific embodiments, the antibody may be, for example, targeted to an epitope of a SARS-CoV-2 structural protein other than the spike protein (S) such as the envelope protein (E), the membrane protein (M) or the nucleocapsid protein (N). In some other specific embodiments, the antibody may be targeted to, for example, an epitope on the spike protein (S) but at a site distinct from the RBD. More generally, where the immunotherapeutic protein comprises a cell surface receptor polypeptide other than an ACE2 polypeptide, or a co-receptor polypeptide, or a fragment thereof, the antibody directed against the target virus may be selected from antibodies that are, for example, broadly neutralising against a class/family of viruses (e.g. human immunodeficiency viruses (HIV)), or which are broadly reactive against a class/family of viruses, broadly neutralising of strains of a specific virus type (e.g. bNmAbs which can neutralise multiple HIV-1 strains), and mAbs which are broadly reactive to a specific virus type. As shown hereinafter, it has been found that an immunotherapeutic protein of the present disclosure and an antibody directed against the target virus may synergistically cooperate to enhance CDC killing of cells (e.g. virus-infected cells). Preferably, the antibody may comprise an Fe region component comprising an amino acid substitution at the position corresponding to H429 of the amino acid sequence of the human IgG1 heavy chain polypeptide (EU numbering) such as, for example, an H429X² amino acid substitution, where X² is selected from phenylalanine (H429F), glutamate (H429E), glutamine (H429Q) and serine (H429S).

In a fifth aspect, the present disclosure provides the use of the immunotherapeutic protein of the first or second aspect, for the treatment and/or the prevention of a viral infection in a subject.

In some embodiments, the immunotherapeutic protein may be used with an antibody directed against the target virus. Preferably, the antibody may comprise an Fe region component comprising an amino acid substitution at the position corresponding to H429 of the amino acid sequence of the human IgG1 heavy chain polypeptide (EU numbering) such as, for example, an H429X² amino acid substitution, where X² is selected from phenlyalanine (H429F), glutamate (H429E), glutamine (H429Q) and serine (H429S).

In a sixth aspect, the present disclosure provides the use of an immunotherapeutic protein of the first or second aspect, in the manufacture of a medicament for the treatment and/or the prevention of a viral infection in a subject.

In a seventh aspect, the present disclosure provides a pharmaceutical composition or medicament comprising an immunotherapeutic protein of the first or second aspect, and a pharmaceutically acceptable carrier, diluent and/or excipient.

Such a pharmaceutical composition or medicament may further comprise an antibody directed against the target virus as described in paragraph [0091] above.

In this specification, a number of terms are used which are well known to those skilled in the art. Nevertheless, for the purposes of clarity, a number of these terms are hereinafter defined.

As used herein, the term “Fc region component” is to be understood as referring to a part of the Fc region of an immunoglobulin heavy (H) chain comprising at least a CH3 domain (or at least a CH4 domain where the Fc region component is derived from an IgE or IgM), where H429 is located, but preferably comprising a CH2 and CH3 domain and optionally further comprising an immunoglobulin hinge sequence (which may, in turn, comprise all or a portion of the lower hinge, core hinge and upper hinge sequences), that is capable of forming (eg by dimerisation) an Fc fragment or Fc-like fragment. An “Fc-like fragment” is to be understood as referring to an Fc fragment-like structure, but which comprises fragments or components of the Fe region such as, for example, the CH3 domain (or CH4 domain) alone or a CH3 domain in combination with a CH2 domain and optionally further comprising an immunoglobulin hinge sequence (which may, in turn, comprise all or a portion of the lower hinge, core hinge and upper hinge sequences).

As used herein, the term “% sequence identity” between two amino acid sequences refers to sequence identity percentages understood as having been calculated using a mathematical algorithm such as that described by Karlin S and SF Altschul, Proc Natl Acad Sci USA 87:2264-2268, 1990, and as modified as in Karlin S and SF Altschul, Proc Natl Acad Sci USA 90:5873-5877, 1993. Such an algorithm is incorporated into the NBLAST and XBLAST programs of Altschul S F et al., J Mol Biol 215:403-410, 1990. BLAST protein searches can be performed with the XBLAST program using the default parameters (see ncbi.nlm.nih.gov/BLAST/). To determine the sequence identity percentage between two amino acid sequences, the mathematical algorithm may align the sequences for optimal comparison purposes, and calculate the percent identity between the sequences as a function of the number of identical positions shared by the sequences (i.e. percent identity=number of identical positions/total number of positions (e.g. (overlapping positions)×100).

As used herein, the term “treating” and “treatment” includes prophylaxis as well as the alleviation of established symptoms of a viral infection. As such, the act of “treating” a viral infection therefore includes: (1) preventing or delaying the appearance of clinical symptoms of the viral infection developing in a subject; (2) inhibiting the viral infection (i.e. arresting, reducing or delaying the development of the viral infection or a relapse thereof, in case of a maintenance treatment, or at least one clinical or subclinical symptom thereof); and (3) relieving or attenuating a viral infection (i.e. causing regression of the viral infection or at least one clinical or subclinical symptom thereof).

As used herein, the phrase “manufacture of a medicament” includes the use of one or more immunotherapeutic protein of the first or second aspect directly as the medicament or in any stage of the manufacture of a medicament comprising one or more immunotherapeutic protein of the first or second aspects.

The term “effective amount” is an amount sufficient to effect beneficial or desired clinical results. An effective amount can be administered in one or more administrations. Typically, an effective amount is sufficient for treating a viral infection or otherwise to palliate, ameliorate, stabilise, reverse, slow or delay the progression of a viral infection. By way of example only, an effective amount of an immunotherapeutic protein of the first or second aspects may comprise between about 0.1 and about 250 mg/kg body weight per day, more preferably between about 0.1 and about 100 mg/kg body weight per day and, still more preferably between about 0.1 and about 25 mg/kg body weight per day. However, notwithstanding the above, it will be understood by those skilled in the art that an effective amount may vary and depend upon a variety of factors including the age, body weight, sex and/or health of the subject being treated, the activity of the particular protein, the metabolic stability and length of action of the particular protein, the route and time of administration of the particular protein, the rate of excretion of the particular protein and the severity of, for example, the viral infection being treated.

The immunotherapeutic protein may be administered in combination with one or more additional agent(s) for the treatment of the viral infection being treated. For example, the immunotherapeutic protein may be used in combination with other agents for treating viral infections (e.g. such as an antibody directed against the target virus, as described in paragraph [0091] above) or at least one clinical or subclinical symptom thereof. Where used in combination with other agents, the immunotherapeutic protein can be administered in the same pharmaceutical composition or in separate pharmaceutical compositions. If administered in separate pharmaceutical compositions, the immunotherapeutic protein and the other agent(s) may be administered simultaneously or sequentially in any order (e.g. within seconds or minutes or even hours (e.g. 2 to 48 hours)).

The immunotherapeutic protein may be formulated into a pharmaceutical composition with a pharmaceutically acceptable carrier, diluent and/or excipient. Examples of suitable carriers and diluents are well known to those skilled in the art, and are described in, for example, Remington's Pharmaceutical Sciences, Mack Publishing Co., Easton, PA 1995. Examples of suitable excipients for the various different forms of pharmaceutical compositions described herein may be found in the Handbook of Pharmaceutical Excipients, 2^{nd} Edition, (1994), Edited by A Wade and P J Weller. Examples of suitable carriers include lactose, starch, glucose, methyl cellulose, magnesium stearate, mannitol, sorbitol and the like. Examples of suitable diluents include glycerol and water. The choice of carrier, diluent and/or excipient may be made with regard to the intended route of administration and standard pharmaceutical practice.

A pharmaceutical composition comprising an immunotherapeutic protein of the first or second aspects may further comprise any suitable binders, lubricants, suspending agents, coating agents and solubilising agents. Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilising agents, and even dyes may be provided in the pharmaceutical composition. Examples of preservatives include sodium benzoate, sorbic acid and esters of p-hydroxybenzoic acid. Anti-oxidants and suspending agents may be also used.

A pharmaceutical composition comprising an immunotherapeutic protein of the first or second aspects will typically be adapted for intravenous or subcutaneous administration. As such, a pharmaceutical composition may comprise solutions or emulsions which may be injected into the subject, and which are prepared from sterile or sterilisable solutions. A pharmaceutical composition may be formulated in unit dosage form (i.e. in the form of discrete portions containing a unit dose, or a multiple or sub-unit of a unit dose).

The immunotherapeutic proteins, uses and pharmaceutical composition of the present disclosure are hereinafter further described with reference to the following, non-limiting Examples.

### EXAMPLES

### Example 1 Production of ACE-2-Fc Fusion Proteins and Activity Analysis Methods and Materials

### Constructs, Fusion Polypeptides and Proteins

The amino acid sequence of the human ACE2 polypeptide is available from the European Nucleotide Archive (ENA, European Molecular Biology Laboratory) at Genbank accession no. BAB40370. The ectodomain of the protein (amino acids 19 to 740; shown as SEQ ID NO: 1 in Table 2) is comprised of a catalytic domain and a collectrin domain. Different forms of the ACE2 ectodomain were produced and studied in this example (as depicted in FIG. 1); particularly, a full-length (fl) ACE2 ectodomain (named flACE2; comprising amino acids 19 to 740 of the mature ACE2 polypeptide), a truncated (tr) ACE2 ectodomain (trACE2) comprising amino acids 19 to 615 of the mature ACE2 polypeptide that excludes the collectrin domain, and an enhanced flACE2 ectodomain (EflACE2) comprising a triple mutation within the ACE2 polypeptide that has been reported to improve its binding affinity to the S protein (Chan et al., 2020 supra). These proteins were produced as fusion polypeptide chains with an Fc component (e.g. an Fc region) derived from human IgG1 (GenBank Accession no. AXN93652.1), to generate flACE2-Fc (SEQ ID NO: 1), trACE2-Fc (SEQ ID NO: 2), and EflACE2-Fc proteins according to standard techniques.

```
TABLE 2
SEQ ID NO: 1
                                      20 30 40 50 60 70 80
                                ETGST IEEQAKTFLD KFNHEAEDLF YQSSLASWNY NTNITEENVQ NMNNAGDKWS AFLKEQSTLA
                90 100 110 120 130 140 150 160
QMYPLQEIQN LTVKLQLQAL QQNGSSVLSE DKSKRLNTIL NTMSTIYSTG KVCNPDNPQE CLLLEPGLNE IMANSLDYNE
              170 180 190 200 210 220 230 240
RLWAWESWRS EVGKQLRPLY EEYVVLKNEM ARANHYEDYG DYWRGDYEVN GVDGYDYSRG QLIEDVEHTF EEIKPLYEHL
              250 260 270 280 290 300 310 320
HAYVRAKLMN AYPSYISPIG CLPAHLLGDM WGRFWTNLYS LTVPFGQKPN IDVTDAMVDQ AWDAQRIFKE AEKFFVSVGL
              330 340 350 360 370 380 390 400
PNMTQGFWEN SMLTDPGNVQ KAVCHPTAWD LGKGDFRILM CTKVTMDDFL TAHHEMGHIQ YDMAYAAQPF LLRNGANEGF
              410 420 430 440 450 460 470 480
HEAVGEIMSL SAATPKHLKS IGLLSPDFQE DNETEINFLL KQALTIVGTL PFTYMLEKWR WMVFKGEIPK DQWMKKWWEM
              490 500 510 520 530 540 550 560
KREIVGVVEP VPHDETYCDP ASLFHVSNDY SFIRYYTRTL YQFQFQEALC QAAKHEGPLH KCDISNSTEA GQKLFNMLRL
              570 580 590 600 610 620 630 640
GKSEPWTLAL ENVVGAKNMN VRPLLNYFEP LFTWLKDQNK NSFVGWSTDW SPYADQSIKV RISLKSALGD RAYEWNDNEM
              650 660 670 680 690 700 710 720
YLFRSSVAYA MRQYFLKVKN QMILFGEEDV RVANLKPRIS FNFFVTAPKN VSDIIPRTEV EKAIRMSRSR INDAFRLNDN
              730 740 750 760 770 780 790 800
                Fc (EU numbering →) 227 237 247 257 267 277
SLEFLGIQPT LGPPNQPPVS GGGGSTHTCP PCPAPELLGG PSVFLFPPKP KDTLMISRTP EVTCVVVDVS HEDPEVKFNW
              810 820 830 840 850 860 870 880
              287 297 307 317 327 337 347 357
YVDGVEVHNA KTKPREEQYN STYRVVSVLT VLHQDWLNGK EYKCKVSNKA LPAPIEKTIS KAKGQPREPQ VYTLPPSREE
              890 900 910 920 930 940 950 960
              367 377 387 397 407 417 427 437
MTKNQVSLTC LVKGFYPSDI AVEWESNGQP ENNYKTTPPV LDSDGSFFLY SKLTVDKSRW QQGNVFSCSV MHEALHNHYT
970
447
QKSLSLSPGK
Comments: SEQ ID NO: 1 is the predicted sequence of the mature ″flACE2-Fc″ fusion polypeptide.
The full-length (fl) ACE2 ectodomain comprises the catalytic domain and the collectrin (or neck)
domain and sequence proximal to the transmembrane region. The N-terminus sequence ETG is
predicted to remain following cleavage of the following underlined leader sequence
(MGILPSPGMPALLSLVSLLSVLLMGCVAETG; SEQ ID NO: 5) upon secretion. The numbering above the fusion
protein sequence utilises the numbering of ACE2, amino acid residues S19 to S740, then a linking
sequence (GGGGS; SEQ ID NO: 4), followed by a human IgG1 Fc comprising T223-K447 (according to
the EU numbering convention, labeled immediately above the C-terminus Fc sequence) and includes
the CH2 and CH3 domains. The IgG1 Fc amino acid, H429, is shown in bold and underlined (EU
numbering). The ACE2 ectodomain sequence matches that of Accession no. BAB40370. Also, a variant
immunotherapeutic protein ″EflACE2-Fc″ was constructed whereby, the amino acids at positions 27,
79 and 330 were mutated to enhance binding affinity to CoV-2 S as follows: T27Y, L79T and N330Y
(Chan et al., 2020 supra). The IgG1 Fc sequence matches the immunoglobulin gamma 1 constant
region, partial [Homo sapiens] sequence of GenBank Accession no. AXN93652.1.
SEQ ID NO: 2
                                      20 30 40 50 60 70 80
                              ETGST IEEQAKTFLD KFNHEAEDLF YQSSLASWNY NTNITEENVQ NMNNAGDKWS AFLKEQSTLA
                90 100 110 120 130 140 150 160
QMYPLQEIQN LTVKLQLQAL QQNGSSVLSE DKSKRLNTIL NTMSTIYSTG KVCNPDNPQE CLLLEPGLNE IMANSLDYNE
              170 180 190 200 210 220 230 240
RLWAWESWRS EVGKOLRPLY EEYVVLKNEM ARANHYEDYG DYWRGDYEVN GVDGYDYSRG QLIEDVEHTF EEIKPLYEHL
              250 260 270 280 290 300 310 320
HAYVRAKLMN AYPSYISPIG CLPAHLLGDM WGRFWTNLYS LTVPFGQKPN IDVTDAMVDQ AWDAQRIFKE AEKFFVSVGL
              330 340 350 360 370 380 390 400
PNMTQGFWEN SMLTDPGNVQ KAVCHPTAWD LGKGDFRILM CTKVTMDDFL TAHHEMGHIQ YDMAYAAQPF LLRNGANEGF
              410 420 430 440 450 460 470 480
HEAVGEIMSL SAATPKHLKS IGLLSPDFQE DNETEINFLL KQALTIVGTL PFTYMLEKWR WMVFKGEIPK DQWMKKWWEM
              490 500 510 520 530 540 550 560
KREIVGVVEP VPHDETYCDP ASLFHVSNDY SFIRYYTRTL YQFQFQEALC QAAKHEGPLH KCDISNSTEA GOKLENMLRL
              570 580 590 600 610 620 630 640
                                                                                                  Fc (EU numbering →)
GKSEPWTLAL ENVVGAKNMN VRPLLNYFEP LFTWLKDQNK NSFVGWSTDW SPYADGSGSG SGTHTCPPCP APELLGGPSV
              650 660 670 680 690 700 710 720
              250 260 270 280 290 300 310 320
FLFPPKPKDT LMISRTPEVT CVVVDVSHED PEVKFNWYVD GVEVHNAKTK PREEQYNSTY RVVSVLTVLH QDWLNGKEYK
              730 740 750 760 770 780 790 800
              330 340 350 360 370 380 390 400
CKVSNKALPA PIEKTISKAK GOPREPOVYT LPPSREEMTK NOVSLTCLVK GFYPSDIAVE WESNGOPENN YKTTPPVLDS
              810 820 830 840 847
              410 420 430 440 447
DGSFFLYSKL TVDKSRWQQG NVFSCSVMHE ALHNHYTOKS LSLSPGK
Comments: SEQ ID NO: 2 is the predicted sequence of the mature ″trACE2-Fc″ fusion polypeptide.
The N-terminus sequence ETG is predicted to remain following cleavage of the following underlined
leader sequence (MGILPSPGMPALLSLVSLLSVLLMGCVAETG; SEQ ID NO: 5) upon secretion. The
numbering above the fusion protein sequence utilises the numbering of ACE2, amino acid residues
S19 to D615, then a linking sequence (GSGSGSG; SEQ ID NO: 6), followed by a human IgG1 Fc, and
which, according to the EU numbering convention (labeled immediately above the C-terminus Fc
sequence), comprises T223-K447. The IgG1 Fc amino acid, H429, is shown bold and underlined. The
truncated ACE2 ectodomain sequence matches that of Accession no. BAB40370. The IgG1 Fc sequence
matches the immunoglobulin gamma 1 constant region, partial [Homo sapiens] sequence of Accession
no. AXN93652.1.
```

For example, a construct encoding the trACE2 ectodomain in pcDNA3.4 (Thermo Fisher Scientific) was prepared by joining a polynucleotide sequence encoding the trACE2 ectodomain to a synthetic sequence encoding a linker and a sequence encoding human IgG1 Fc (particularly, IgG1 Fc having the amino acid sequence of GenBank accession no. AXN93652.1 (immunoglobulin gamma 1 constant region, partial [Homo sapiens]; National Center for Biotechnology Information (NCBI) database). For the generation of a flACE2-Fc expression construct, a KpnI digestion of the trACE2 construct was conducted followed by insertion of a codon-optimised polynucleotide sequence encoding the ACE2 collectrin domain (GeneArt, Thermo Fisher Scientific). For the generation of an EflACE2-Fc expression construct, a synthetic polynucleotide sequence equivalent to that encoding flACE2-Fc, but with three mutations (i.e. T27Y, L79T and N330Y; sACE2.v2.4 described by Chan et al., 2020 supra) was used. In addition, variants of the fusion proteins were produced that incorporated H429F and H429Y mutations in the Fc region introduced using cleavage at a unique AfeI site within the IgG Fc-encoding sequence, and the subsequent insertion of appropriate mutagenic oligonucleotides using NEBuilder (New England Biolabs, Ipswich, MA, United States of America) according to the manufacturer's instructions.

Expression of the fusion proteins was conducted using transient transfection of Expi293 cells (Thermo Fisher Scientific). The supernatant of the Expi293 cells transiently transfected for the expression of flACE2-Fc-WT (i.e. where the Fc region was according to the wild type (WT) hIgG1 Fc mentioned in the preceding paragraph) was extensively dialysed against 10 mM Tris-HCl pH 8 (buffer A) and applied to a High-Q column (Bio-Rad Laboratories, Hercules, CA, United States of America). Bound proteins were eluted with a linear gradient to buffer A with 0.4 M NaCl and washed with 1 M NaCl. Fractions were examined by SDS-PAGE, and those fractions containing the flACE2-Fc-WT fusion protein were pooled and concentrated using a 30 kDa cut-off filtration device (Pall Corporation, Port Washington, NY, United States of America) and separated by size-exclusion chromatography (SEC) using a Superose 6 column (GE Life Sciences, Chicago, IL, United States of America).

The SARS-CoV-2 RBD-Ig and RBD AviTag have been described previously (see Juno J A et al., Nat Med 26:1428-1434, 2020, Lopez E et al., JCI Insight. 6(16):e150012, 2021 and Hartley G E et al., Sci Immunol 5, 2020, respectively). The RBD AviTag was biotinylated in vitro using BirA ligase or in situ using Expi293BirA cells (Wines B D et al., J Immunol 197(4):1507-1516, 2016).

Lamelli native PAGE (N-PAGE), 150V, 2.5 h, 4° C., was according to Wines B D et al., J Immunol 162(4):2146-2153, 1999.

### Virus Neutralisation Assays

Antiviral titre was determined using SARS-CoV-2 (CoV/Australia/VIC01/2020) in a micro-neutralisation assay as described previously (Juno J A et al., Nat Med 26(9):1428-1434, 2020).

### Bio-Layer Interferometry

Measurements of the affinity of the ACE2-Fc fusion proteins for the CoV-2 S RBD were performed on the Octet RED96e (FortéBio, Fremont, CA, United States of America). All assays were performed at 25° C. using anti-human IgG Fc capture (AHC) biosensor tips (FortéBio) in kinetics buffer (PBS pH 7.4 supplemented with 0.1% (w/v) BSA and 0.05% (v/v) TWEEN-20). After a 60 second (60 s) biosensor baseline step, the fusion proteins (20 μg/mL) were loaded onto anti-human IgG Fc capture (AHC) biosensors by submerging sensor tips for 200 s and then washing in kinetics buffer for 60 s. For most of the fusion proteins, association measurements were performed by dipping into a two-fold dilution series of SARS-CoV-2 RBD from 16-250 or 500 nM for 180 s and then measuring dissociation in kinetics buffer for 180 s. For EflACE2-Fc WT, a two-fold dilution series of 2-31 or 63 nM was used. The biosensor tips were regenerated five times using a cycle of 5 s in 10 mM glycine pH 1.5 and 5 s in kinetics buffer, and baseline drift was corrected by subtracting the average shift of a fusion protein-loaded sensor not incubated with SARS-CoV-2 RBD, and an unloaded sensor incubated with SARS-CoV-2 RBD. Curve fitting analysis was performed with Octet Data Analysis 10.0 software using a global fit 1:1 model to determine K_{D} values and kinetic parameters. Curves that could not be fitted were excluded from the analyses. Kinetic constants reported were representative of two independent experiments.

ACE2-Fc Fusion Proteins and Dimeric rsFcγR Binding by Flow Cytometry

The ACE2-Fc fusion proteins or rituximab (a chimeric mAb targeted to CD20), at 5 μg/ml, or the indicated concentrations were incubated with Ramos cells expressing transfected spike protein (Ramos-S cells; Lee W S et al., medRxiv doi:10.1101/2020.12.13.20248143, 2020) at 5×10⁶ cells/ml in 25 μl of FACS buffer (PBS containing 0.5% BSA, 1 mM glucose) for 30 min on ice, and then washed twice with FACS buffer, incubated with PE or FITC conjugated anti-human IgG-Fc for 30 min on ice, before being washed again and resuspended in 25 μl of FACS buffer.

Evaluation of binding of dimeric recombinant soluble FcγR (rsFcγR) was performed based on work previously described (Wines et al., 2016 supra). ACE2-Fc opsonised Ramos-S cells were resuspended in 0.5 μg/ml of biotinylated dimeric rsFcγRIIa (H131 allelic form) or dimeric rsFcγRIIla (V158 form) or FACS buffer and incubated for 30 min on ice followed by 1/500 streptavidin-APC (or anti-hIgG-Fc FITC for confirmation of ACE2-Fc opsonisation) for 20 min on ice. The cells were washed, resuspended in FACS buffer and analysed on a BD FACSCanto™ II flow cytometer (Becton Dickinson, Franklin Lakes, NJ, United States of America).

### RBD Variant ACE2-Fc Inhibition Multiplex Assay

A custom coronavirus multiplex array (Lopez E et al., JCI Insight. 6(16):e150012, 2021) was performed using the SARS-CoV-2 S1, the SARS-CoV-1 Si subunit (ACRObiosystems, Newark, DE, United States of America), the HCoV NL63 S1 and S2 subunits (Sino Biological Inc., Beijing, China), NL63 S trimer (BPS Bioscience, San Diego, United States of America), and hexahistidine tagged RBD WT (SARS CoV-2, isolate 2019-nCov, NCBI Ref: NC_045512.2, aa residues 334-527) and 24 variants identified from the GISAID RBD surveillance repository expressed from pcDNA3 (GenScript) in HEK293 Expi cells (Thermo Fisher Scientific) and purified by affinity chromatography. The multiplex bead coupling, experimental steps, and data acquisition on a FlexMap3D™ (Luminex Corporation, Austin, TX, United States of America) analyser was as previously described (Lee W S et al., 2020 supra, and Wheatley A K et al., Nature Comm 12, Article number:1162, 2021). Briefly, trACE2-Fc was biotinylated using EZ-Link® Sulfo-NHS-LC-Biotin (Thermo Fisher Scientific) according to the manufacturer's instructions. The trACE2-Fc-biotin (70 nM) was incubated with a two-fold concentration series (2.2 to 282 nM) of the decoy fusion proteins, trACE2-Fc and flACE2-Fc, and the affinity-enhanced EflACE2-Fc, or the inhibitory mAb S35 (ACROBiosystems, Newark, DE, United States of America), along with the above-described coronavirus multiplex beads in 384 well plates, for 2 hours on a plate shaker at RT. The beads were then washed twice in PBS, 0.05% (w/v) Tween 20 then 40 μl of R-Phycoerythrin conjugated streptavidin (SAPE; Thermo Fisher Scientific) at 4 μg/ml was then added for 1 hour. This was followed by a further incubation for 1 hour after the addition of 10 μl of 10 μg/ml of R-Phycoerythrin Biotin-XX conjugate (Thermo Fisher Scientific) for the purpose of amplifying the signal. Plates were then washed three times, and 60 μl of sheath buffer was added to each well, with plates left to shake for 10 minutes on a plate shaker prior to acquisition on a FlexMap3D™ (Luminex Corporation) analyser. The binding of phycoerythrin-labelled reporter is measured as MFI (Median Fluorescence Intensity). For inhibition by trACE2-Fc and flACE2-Fc, values were divided by 0.8 to account for a maximum possible inhibition of 80% (NB IC50 (nM) values are reported).

### Complement Dependent Cytotoxicity (CDC) Assay

CDC was measured by opsonising Ramos-S cells as above (5×10⁶ cells/ml in 25 μl of FACS buffer for 30 minutes on ice) before resuspending in 1/3 diluted normal human serum for 30 minutes at 37° C. Cells were washed twice with PBS and the dead cells were then enumerated by staining with 1/500 Zombie Green (BioLegend, San Diego, CA, United States of America) before fixing with 2% paraformaldehyde in PBS and analysis on a BD FACSCanto™ LI flow cytometer.

### Complement Fixation Immunoassay for ACE2-Fc Fusion Proteins

Complement fixation immunoassays were performed by ELISA wherein the 96-well plates were coated with 5 μg/ml avidin in PBS overnight, blocked, and then incubated with either serially two-fold titrated or at a single concentration (2.5 μg/ml) biotinylated RBD in 0.1% casein for 1 hr at RT. The ACE2-Fc fusion proteins were then added at 2 μg/ml or titrated over an indicated concentration range. C1q fixation or the formation of the membrane attack complex (MAC) was detected as described in Kurtovic L et al., BMC Med 17:45, 2019.

FcγRIIIa-NF-κB-RE nanoluciferase reporter assay

FcγRIIIa-NF-κB-RE nanoluciferase reporter assays were conducted using IIA1.6/FcR-γ/FcγRIIIa V158 cells expressing a NF-κB response element-driven nanoluciferase (NanoLuc, pNL3.2.NF-κB-RE[NlucP/NF-κB-RE/Hygro], Promega Corporation, Madison, WI, United States of America), and was performed essentially as previously described (Lee et al., 2020 supra). Briefly, Ramos cells expressing Spike-IRES-orange2 were used as target cells and were incubated with agonists and the FcγRIIIa/NF-κB-RE reporter cells for 5 hours before measurement of induced nanoluciferase with Nano-Glo substrate (Promega).

### Results and Discussion

### ACE2-Fc Fusion Protein Construction and Production

A series of ACE2-Fc fusion proteins (Table 3) were produced and analysed for their capacity to neutralise SARS-CoV-2 infection and mediate Fe-dependent effector functions normally attributed to the mechanisms of action of antibodies.

TABLE 3

| | | ACE2 | IgG1 Fc |
|---|---|---|---|
| | ACE2 ectodomain | mutation/ | mutation/modification |
| Protein name | form (sequence) | modification | (EU numbering) |
| trACE2 | Truncated ACE2 | Not mutated | N/A* |
| | (amino acids 19-615) | | |
| trACE2-Fc-WT | Truncated ACE2 | Not mutated | Not modified |
| | (amino acids 19-615) | | |
| trACE2-Fc-H429F | Truncated ACE2 | Not mutated | His 429 Phe |
| | (amino acids 19-615) | | |
| trACE2-Fc-H429Y | Truncated ACE2 | Not mutated | His 429 Tyr |
| | (amino acids 19-615) | | |
| trACE2-Fc-kif | Truncated ACE2 | Not mutated/ | Modified N-glycan at |
| | (amino acids 19-615) | modified N- | Asn 297 |
| | | glycans | |
| flACE2-Fc-WT | Full-length ACE2 | Not mutated | Not modified |
| | (amino acids 19-740) | | |
| flACE2-Fc-H429F | Full-length ACE2 | Not mutated | His 429 Phe |
| | (amino acids 19-740) | | |
| flACE2-Fc-H429Y | Full-length ACE2 | Not mutated | His 429 Tyr |
| | (amino acids 19-740) | | |
| EflACE2-Fc-WT | Full-length ACE2 | Thr 27 Tyr | Not modified |
| | (amino acids 19-740) | Leu 79 Thr | |
| | | Asn 330 Tyr | |
| EflACE2-Fc-H429F | Full-length ACE2 | Thr 27 Tyr | His 429 Phe |
| | (amino acids 19-740) | Leu 79 Thr | |
| | | Asn 330 Tyr | |
| EflACE2-Fc-H429Y | Full-length ACE2 | Thr 27 Tyr | His 429 Tyr |
| | (amino acids 19-740) | Leu 79 Thr | |
| | | Asn 330 Tyr | |

*N/A: Not applicable as no Fc present (i.e. truncated ectodomain only)

In an attempt to enhance the avidity of binding to the SARS-CoV-2 spike protein or to confer and improve Fc-dependent effector functions, three versions of the ACE2 ectodomain were fused to an Fc region of IgG1 (FIG. 1) either unmodified or altered by mutation (i.e. substitution of histidine 429 with phenylalanine (H429F) or tyrosine (H429Y)) or by modified glycosylation (i.e. a high mannose glycan lacking core fucose; trACE2-Fc-kif).

### ACE2-Fc Fusion Protein Purification

Proteins were produced in Expi293 cells and purified by (an) ion exchange (IEX) then by size exclusion chromatography (SEC) at pH 7.4 (FIG. 2). All of the fusion proteins showed a similar IEX purification profile to that of flACE2-Fc (FIG. 2A) which contained a major elution peak (peak *; FIG. 2A), and SDS-PAGE analysis of the collected fractions showed a single major species of about 270 kDa (FIG. 2B). Except for those fusion proteins comprising the H429Y mutation, SEC analysis of the major IEX peak confirmed the presence of a major monomeric species (NB the monomer species are considered to be single molecules (i.e. monomer molecules) comprising two (dimerised) copies of the respective ACE2-Fc fusion polypeptide self-associated through the Fc region components). For further analysis, the major monomeric species resolved by SEC was collected so as to exclude higher molecular weight oligomers and other impurities (FIG. 2C). In contrast, in SEC analysis, the fusion proteins comprising the H429Y mutation (i.e. of the major IEX peak from flACE2-Fc-H429Y mutant (FIG. 2D), and also of the major IEX peaks from the trACE2-Fc-H429Y and EflACE2-Fc-H429Y proteins (not shown)), revealed the presence of significant quantities of oligomeric (Yₒₗᵢ) and monomeric (Yₘₙ) species. The oligomeric and monomeric species were subsequently evaluated separately for functional activity along with the other mutated and WT ACE2-Fc fusion proteins.

Affinity of ACE2-Fc Fusion Protein Interaction with SARS CoV-2 S RBD

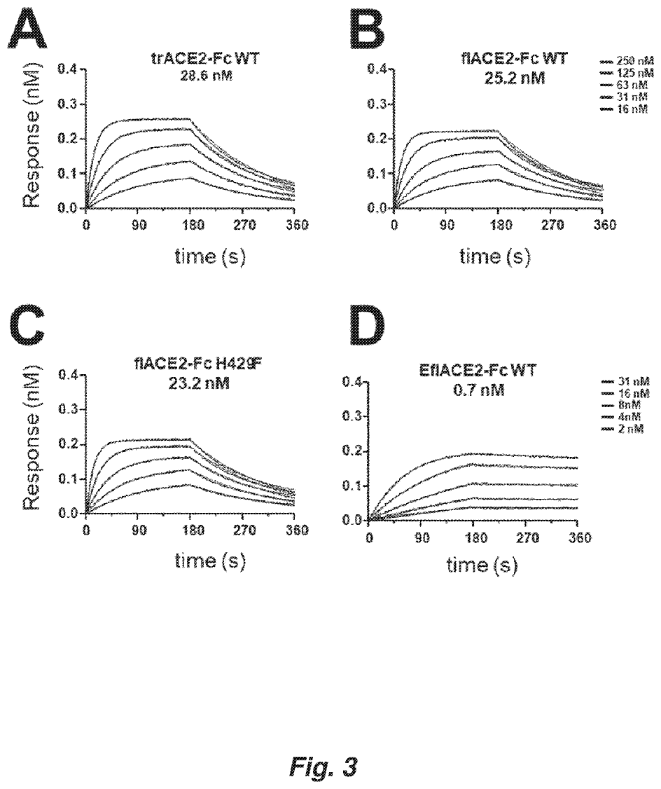

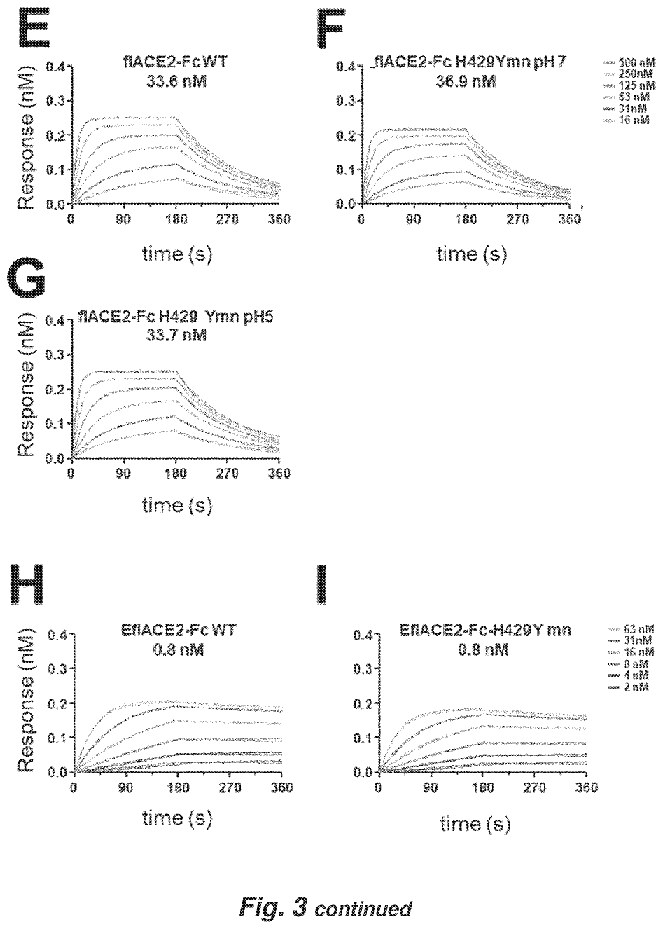

Using biolayer inferometry (with immobilised ACE2-Fc fusion proteins), it was found that only the triple mutation of ACE2 in the EflACE2-Fc fusion protein increased the intrinsic affinity for the spike protein RBD (FIG. 3). Importantly, modification of the IgG Fc region did not affect the interaction affinities; with the mutated Fc region components exhibiting affinities equivalent to their wildtype (WT) Fc region counterparts. Thus, K_{D} values were similar for the trACE2-Fc-WT (28.6 nM) (FIG. 3A) and flACE2-Fc-WT 25.2-33.6 nM (FIG. 3B, 3E), but the EflACE2-Fc-WT protein showed a ˜30-40 fold increase in affinity to K_{D}=0.7-0.8 nM (FIG. 3H). Further, the mutated Fc proteins, and flACE2-Fc-H429F (FIG. 3C) showed the same level of affinity as the non-mutated trACE2-Fc-WT and flACE2-Fc-WT (FIGS. 3A and 3B, respectively). Similarly, the affinity of the flACE2-Fc-H429Y fusion protein, purified at pH 7.4 or pH 5.0 (FIG. 3F, 3G) was identical to that of flACE2-Fc-WT (FIG. 3E), and the same sub-nanomolar (0.8 nM) affinity was also apparent with both the EflACE2-Fc-H249Y (FIG. 3I) and EflACE2-Fc-WT (FIG. 3H) fusion proteins.

### SARS-CoV-2 RBD-Ig Binding Activities of ACE2-Fc Fusion Proteins

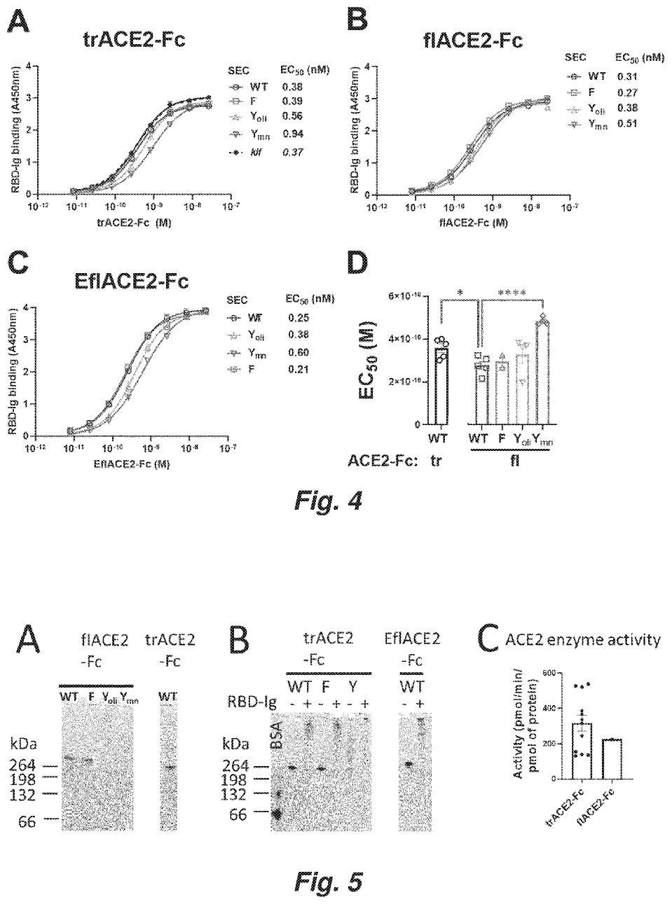

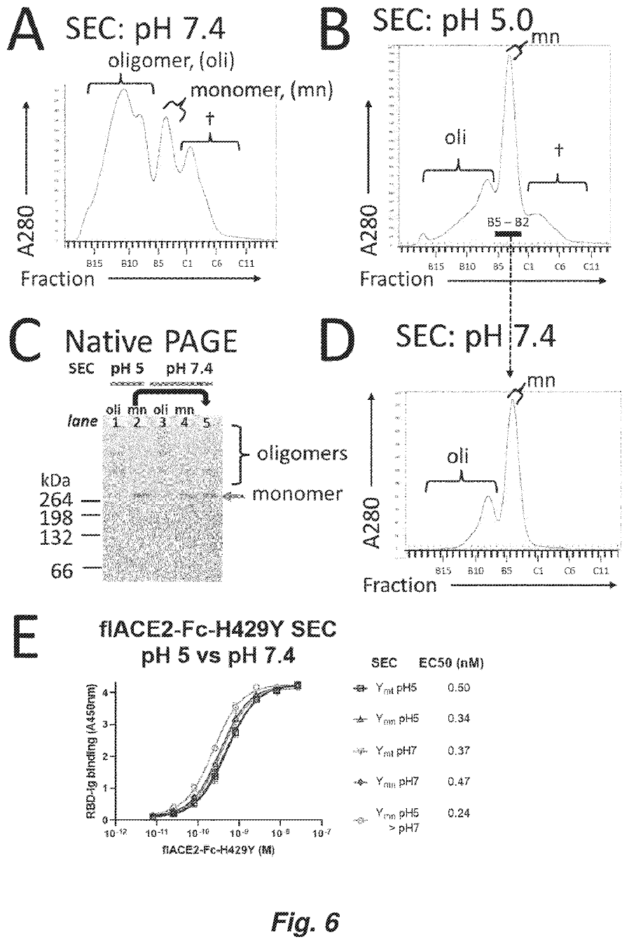

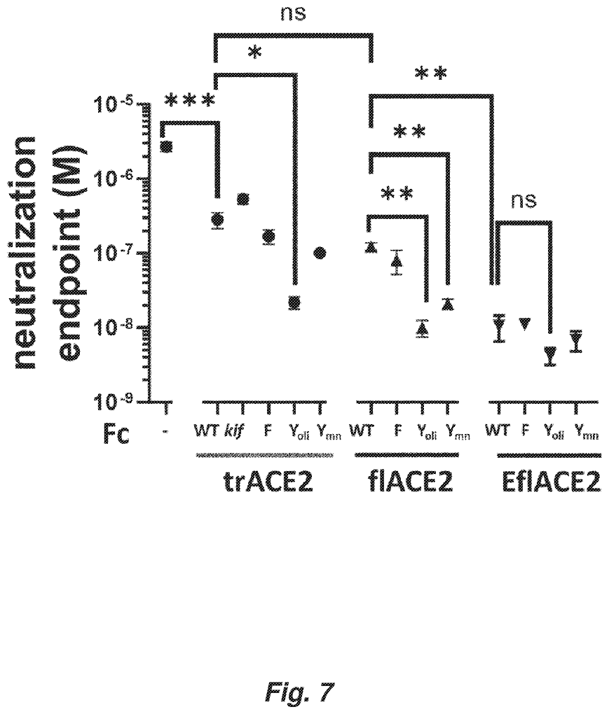

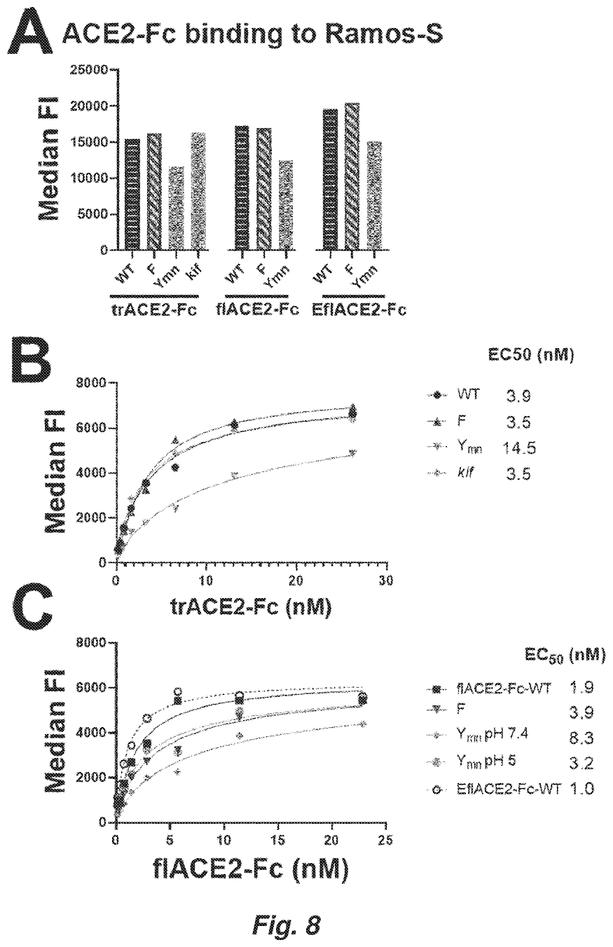

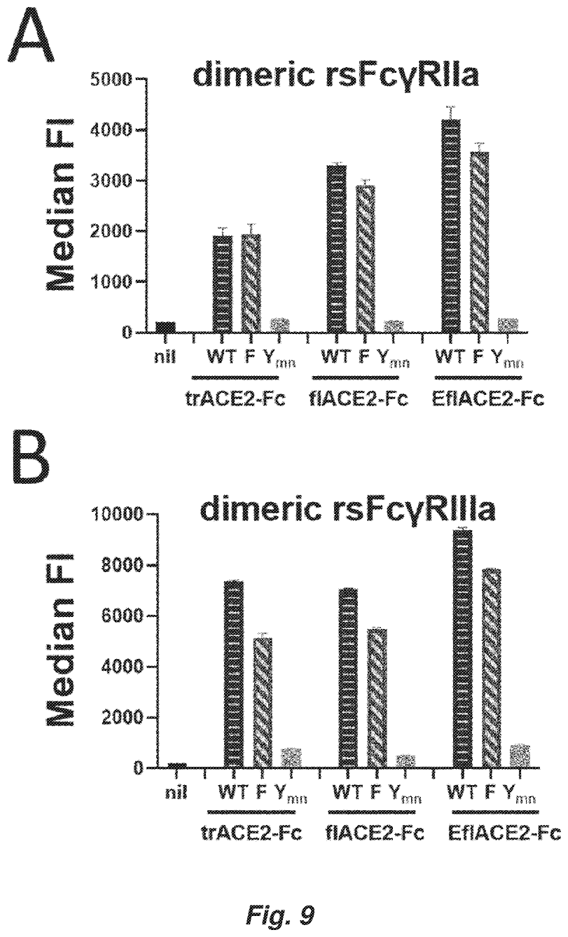

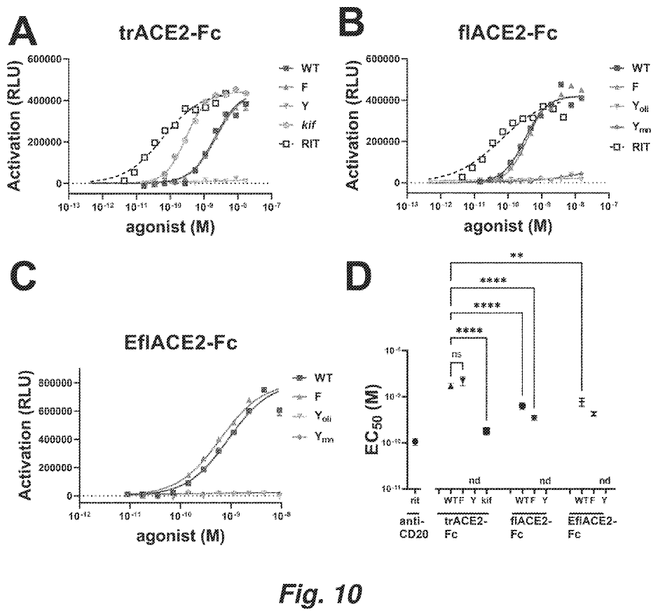

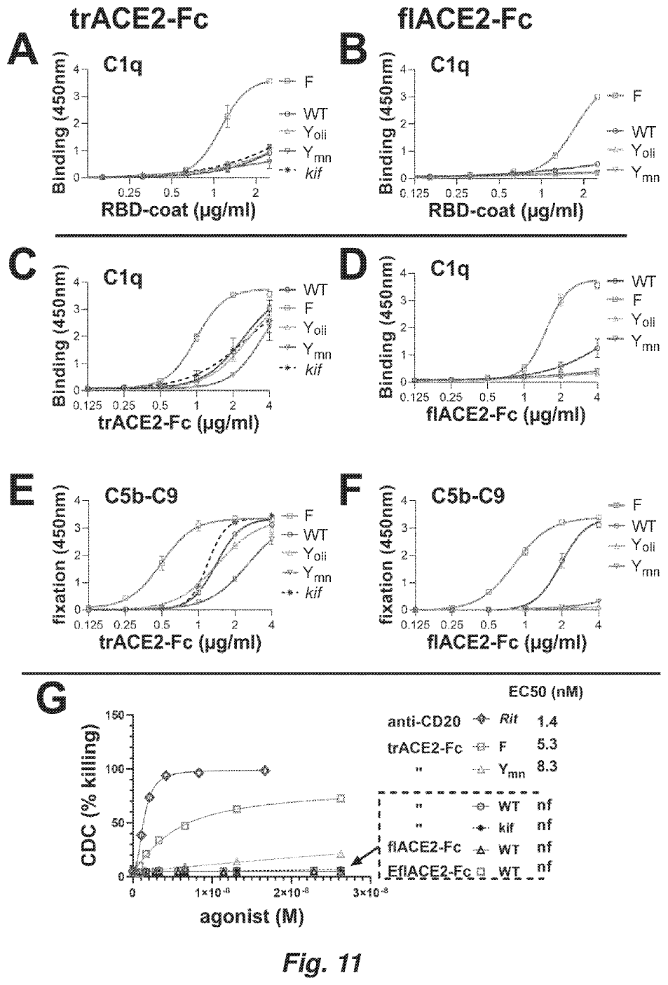

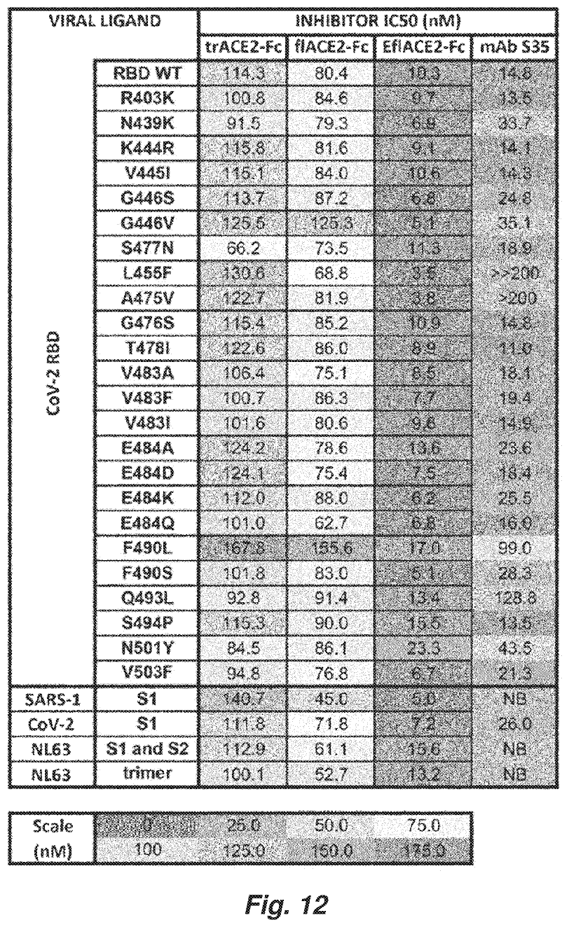

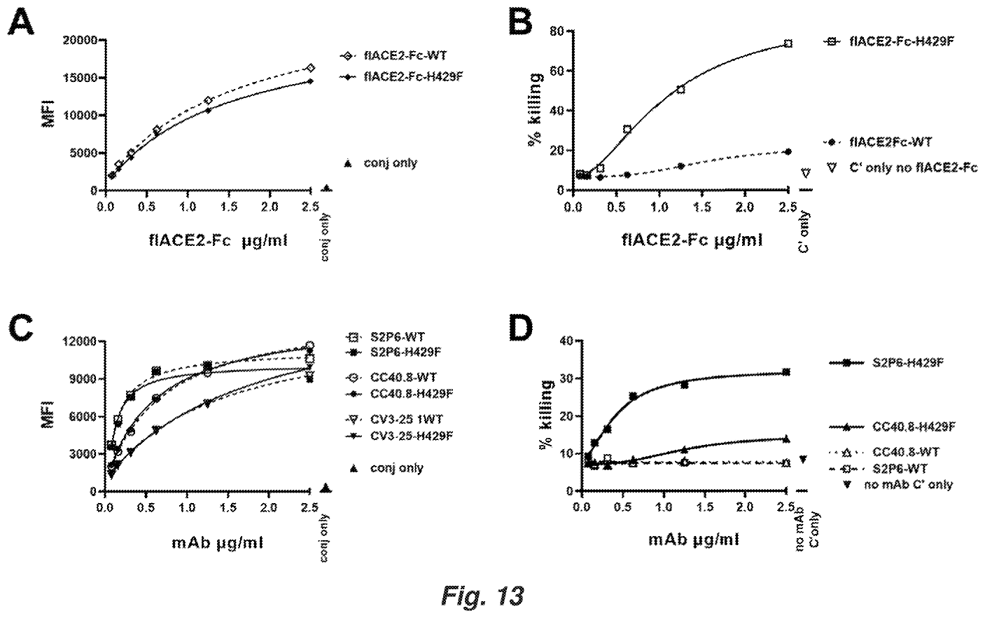

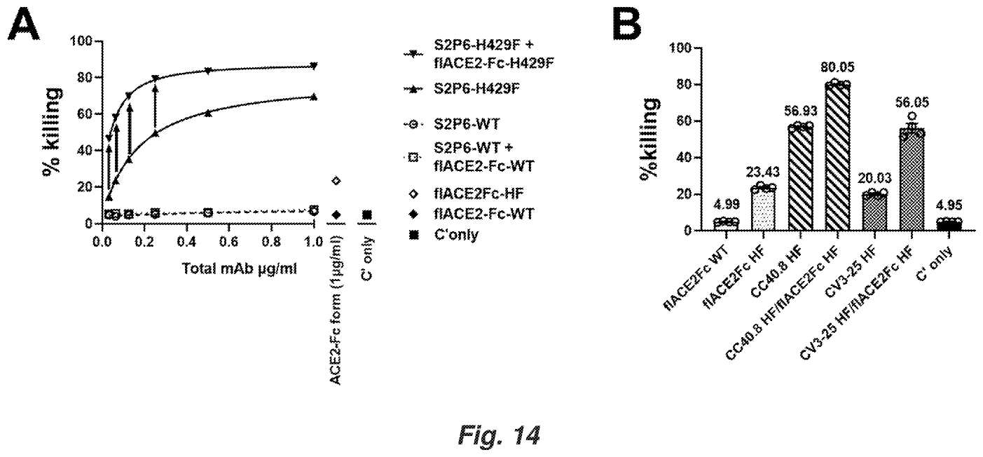

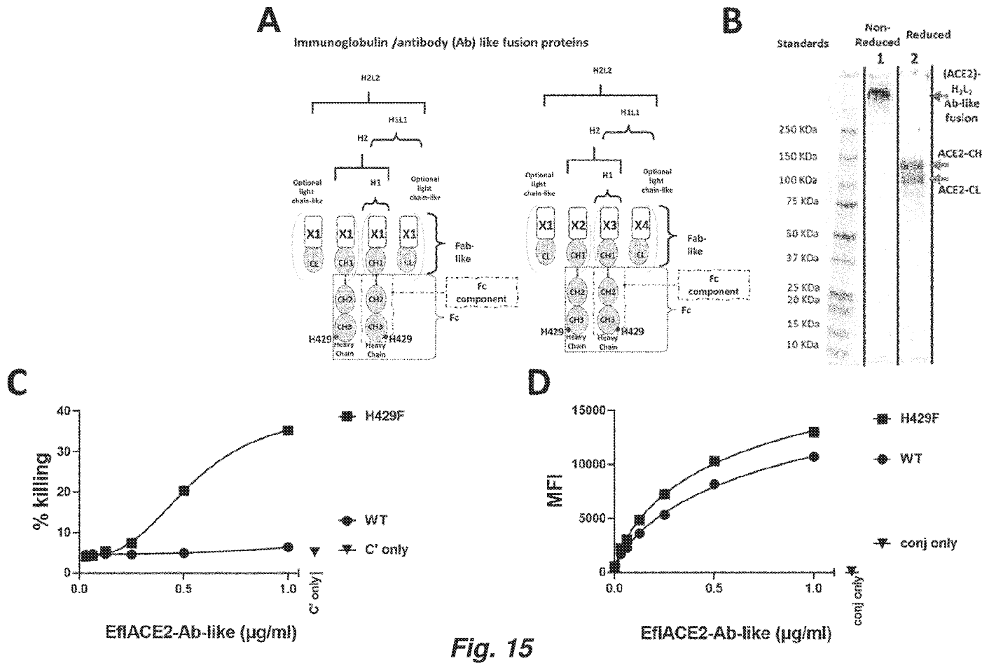

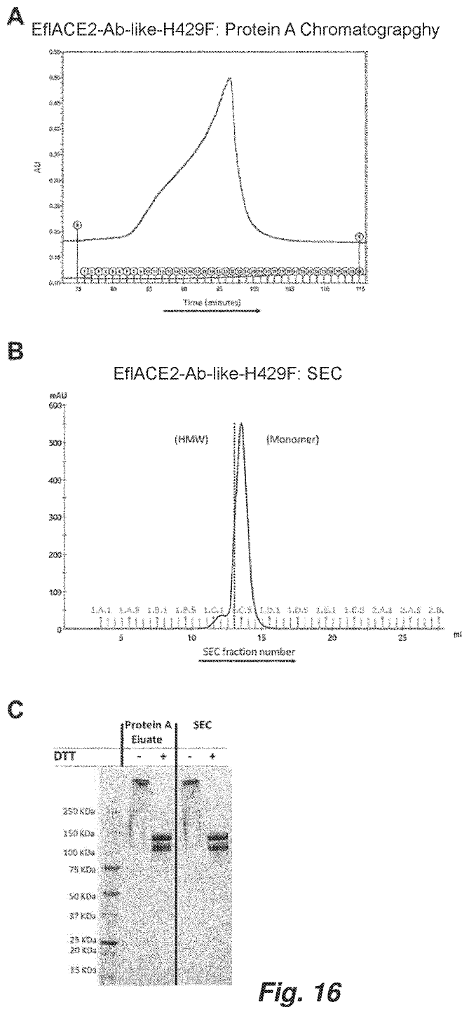

The binding of the trACE2-Fc-WT, flACE2-Fc-WT and EflACE2-Fc-WT fusion proteins to SARS-CoV-2 receptor binding domain (RBD)-Ig was evaluated by ELISA (FIG. 4A-C) and found to be similar overall (i.e. EC₅₀ 0.35 nM, 0.27 nM and 0.25 nM, respectively; FIG. 4D). The enhanced intrinsic affinity of the EflACE2-Fc, determined by BLI analysis (FIG. 3D, H, I), was less apparent with binding to this bivalent form of RBD presented as a fusion with mouse IgG1 Fc (RBD-Ig). The binding activity of the various fusion proteins with mutated Fe regions were also equivalent, excepting the flACE2-Fc-H429Y which was of slightly lower apparent affinity (EC₅₀ 0.48 nM) and the other H429Y Fc variants likewise trending to a lower apparent affinity (FIG. 4).

### Native PAGE (N-PAGE) and Gel-Shift Analysis of ACE2-Fc:RBD Interaction

Native PAGE (N-PAGE) analysis showed that ACE-2-Fc WT fusion proteins migrate as a single species; for trACE2-Fc at ˜ 264 kDa and >264 kDa for the flACE2-Fc and EflACE2-Fc proteins (FIG. 5A, 5B) reflecting the additional presence of the collectrin domain in flACE2-Fc and EflACE2-Fc proteins. Interestingly, the fusion proteins with the mutant H429Y Fc region, flACE2-Fc-H429Y (FIG. 5A, lanes Yₒₗᵢ and Yₘₙ; i.e. oligomer peak and notional monomer peak by SEC) and similarly trACE2-Fc-H429Y (FIG. 5B, lane Y−), but not the proteins with wild type (WT) or other mutated Fc regions, migrated in N-PAGE as several different species that were not apparent by denaturing SDS-PAGE (FIG. 2B), suggesting that the SEC-purified monomeric species of ACE2-Fc H429Y (comprising two copies of the fusion polypeptide self-associated through the Fc region components) (FIG. 2D) may re-form oligomeric species which were also present in the IEX-purified protein preparation (FIG. 2D) and (FIG. 5A lane Yₒₗᵢ).

Gel shift analysis by N-PAGE (FIG. 5B) showed that the trACE2-Fe, flACE2-Fc and EflACE2-Fc WT fusion proteins and the variants including mutated H429Y Fc regions, displayed high specific binding activity for CoV-2 RBD as visualised by the nearly entire shift in migration to a high molecular weight complex following interaction with CoV-2 RBD-Ig. The similar ACE2 enzymatic activity of the trACE2-Fc-WT and flACE2-Fc-WT fusion proteins (FIG. 5C) indicates that the fusion of the Fc region to the ACE2 polypeptide does not impair the carboxypeptidase activity of the fusion protein (indicating that the ACE2 conformation has been preserved).

### Oligomerisation of the ACE2-Fc H429Y Fusion Protein is pH Dependent

Oligomerisation of fusion proteins with a mutated H429Y Fe region was examined by SEC separation at pH 5.0 of the flACE2-Fc-H429Y prepared by IEX. In contrast to SEC at pH 7.4 (FIG. 2D and FIG. 6A), SEC at pH 5.0 (FIG. 6B) revealed a greater proportion of monomer Yₘₙ which N-PAGE showed was purified to homogeneity (FIG. 6C: lane 2 cf lane 1). The purified monomeric flACE2-FC—H429Yₘₙ, pH 5.0, was re-analysed by dialysis at pH 7.4 followed by both N-PAGE (FIG. 6C) and by SEC at pH 7.4 (FIG. 6D). Re-exposure to pH 7.4 yielded a mix of oligomer Yₒₗᵢ and monomer Yₘₙ species (FIG. 6C; lane 2 pH 5.0 cf lane 5 pH 7.4) indicating some exchange between these forms occurs at physiological pH. The flACE2-Fc-H429Yₘₙ prepared at pH 5.0 showed equivalent binding to RBD-Ig as the other flACE2-Fc WT fusion proteins and Fe variants (FIG. 6E). It is also clear that the prevalence of oligomers is related to the tyrosine substitution of histidine 429 as only a trace of oligomer was observed in the fusion proteins with phenylalanine-substituted H429F Fc region (not shown).

### Evaluation of Virus Neutralisation Potency

The antiviral activities of the ACE2-Fc fusion proteins was determined in a micro-neutralisation assay of SARS-CoV-2 infection of Vero cells (FIG. 7) where the EC₅₀ endpoint corresponds to neutralisation of ˜99% of the inoculum virions (Khoury D S et al., Nat Rev Immunol 20(12):727-738, 2020).

The SARS-CoV-2 neutralisation endpoint of the truncated ectodomain, trACE2 (2.70 μM), was improved ˜10-fold by fusion to the wild type Fc region of IgG1 (trACE2-Fc-WT, 283 nM) (FIG. 7). The improved potency is consistent with improved avidity of binding to SARS-CoV-2 spike RBD because of ACE2-Fc bivalency resulting from fusion of ACE2 ectodomains to the IgG Fc region and was also similar to that of flACE2-Fc-WT. The EflACE2-Fc-WT showed a further ˜20-fold improvement (11 nM) over the flACE2-Fc-WT and trACE2-Fc-WT fusion proteins and ˜200-fold more than un-fused trACE2; which is consistent with its enhanced, 35-fold increase in affinity for the SARS-CoV-2 spike protein RBD (FIG. 3D) resulting from the triple mutation of the ACE2 component of the fusion protein compared to unmodified flACE2 (e.g. FIG. 3B).

Analysis of the Fc modifications revealed several interesting differences. First, the H429Y mutation in trACE2-Fc and flACE2-Fc improved their virus neutralisation potency (FIG. 7). Secondly, it was found that the neutralisation activity (21.9 nM) of the oligomeric form of trACE2-Fc-H429Yₒₗᵢ isolated by SEC at pH 7.4 was enhanced 13-fold over the trACE2-Fc-WT. Both the oligomer, Yₒₗᵢ (FIG. 1E) and the notional monomer, Yₘₙ (FIG. 1B), forms of flACE2-Fc-H429Y (FIG. 2D) (endpoints of 10.0 and 20.9 nM respectively; FIG. 7), showed greater potency than the flACE2-Fc-WT (124 nM) fusion protein and were similar to the viral neutralisation achieved with EflACE2-Fc-WT (10.6 nM), which represents a neutralisation activity that is over 200-fold greater than that of monomeric trACE2 (FIG. 7). Thus, the Fc-H429Y mutation in the CH3 domain of the Fc region components which facilitates oligomerisation of the fusion proteins increased SARS-CoV-2 neutralisation in the trACE2-Fc-H429Y and flACE2-Fc-H429Y fusion protein formats, and also trended towards a greater level of potency when combined with the triple mutation of the ACE2 in the intrinsically higher affinity EflACE2-Fc-H429Y (4.23 nM), the latter representing a ˜600-fold overall increase in neutralisation activity over the monomeric trACE2 (FIG. 7). Accordingly, a CH3 domain with this mutation may be considered as an oligomerisation domain.

The phenylalanine substitution of histidine 429 (H429F) of the ACE2-Fc fusion proteins did not enhance neutralisation.

Interaction of ACE2-Fc Fusion Proteins with SARS-CoV-2 Spike Protein on the Cell Surface

Binding of the ACE2-Fc fusion proteins to SARS-CoV-2 spike protein on the cell surface was evaluated by flow cytometry using Ramos cells expressing CoV-2 spike protein (Ramos-S cells; Lee W S et al., medRxiv doi:10.1101/2020.12.13.20248143, 2020) (FIG. 8). All ACE2-Fc fusion proteins bound to the Ramos-S cells (FIG. 8A) with all, except those fusion proteins comprising a mutated H429Y Fc region, showing similar binding profiles in the range 1.0-3.9 nM (FIG. 8B, 8C). The trACE2-Fc-H429Yₘₙ and flACE2-Fc-H429Yₘₙ purified at pH 7.4 showed reductions in binding efficiency to EC₅₀=14 nM and 8 nM respectively, but the flACE2-Fc purified at pH 5.0 (FIG. 8C) had similar binding to that of flACE2-Fc-WT. Despite its 30-fold improved intrinsic affinity for SARS-CoV-2 RBD (FIG. 3D), EflACE2-Fc showed only modest improvement in binding to the cell-expressed spike protein compared to that of the unmodified flACE2-Fc-WT (FIG. 8C), which suggests that the bivalency arising from the Fe region fusion confers potent binding avidity for the spike protein when arrayed on the surface of the cell or for RBD-Ig, when arrayed on the surface of an ELISA plate as was also found in ELISA analysis of binding (FIG. 4).

Interaction of ACE2 Fusion Proteins with IgG FcγR

The interaction of FcγRIIa and FcγRIIIa with the ACE2-Fc fusion proteins was evaluated by flow cytometry using ACE2-Fc opsonised Ramos-S cells (Lee W S et al. 2020 supra) and dimeric recombinant soluble FcγR (Wines et al., 2016 supra). The trACE2-Fc, flACE2-Fc and EflACE2-Fc fusion proteins all bound to FcγRIIa and FcγRIIIa (FIG. 9A, 9B), however, the variant proteins comprising a mutated H429Y Fc region largely failed to bind to either of these Fc receptor types.

### FcγRIIIa Activation by ACE2 Fc Fusion Proteins

Antibody dependent cytotoxicity (ADCC) and Fc-dependent clearance of viruses are important antiviral effector mechanisms that may play a protective role during SARS-CoV-2 infection (Li D et al., bioRxiv doi: 10.1101/2020.12.31.424729, 2021; and Shafer A et al., J Exp Med 218(3):e20201993, 2021). Thus, in this experimentation, the ability of the trACE2-Fc, flACE2-Fc and EflACE2-Fc WT fusion proteins and variant proteins to activate FcγRIIIa was evaluated. It was found that Ramos-S cells opsonised with ACE2-Fc fusion proteins comprising the wild type Fc region all initiated FcγRIIIa activation (FIG. 10). However, the flACE2-Fc fusion protein induced a greater level of activation than trACE2-Fc indicating that the inclusion of the collectrin domain in the ACE2 component of the fusion protein and/or different linker sequence in the fusion to the Fe component substantially improves the potency of FcγRIIIa activation. Further, it was found that the increased affinity that the EflACE2-Fc fusion protein has for the SARS-CoV-2 spike protein did not alter the level of FcγRIIIa activation which was equivalent to that of flACE2-Fc (FIG. 10D).

Production of the trACE2-Fc fusion protein in the presence of kifunensine (van Berkel P H C et al., Biotechnol Bioeng 105(20:350-357, 2010), that is trACE2-Fc-kif, also substantially improved the modest level of FcγRIIIa activation shown by trACE2-Fc to a level comparable to that of the flACE2-Fc and EflACE2-Fc fusion proteins (FIG. 10D) and approaching that of the therapeutic anti-CD20 mAb, rituximab, on the CD20+Ramos-S cells (FIG. 10A,10 D). Kifunensine, a mannosidase inhibitor, prevents normal N-linked glycosylation, including core fucosylation and, in immunoglobulins, the lack of fucose on the heavy chain glycan at Asn297 is known to improve FcγRIIIa binding and activation (Ferrara C et al., Proc Natl Acad Sci USA 108(31):12669-12674, 2011). Thus, it was hypothesised that similar treatment of the flACE2-Fc and EflACE2-Fc fusion proteins, or amino acid residue substitution to increase affinity for FcγRIIIa (Wang et al., 2018 supra), would be likely to further improve their FcγRIII activating potency.

Modification of the trACE2-Fc, flACE2-Fc and EflACE2-Fc fusion proteins by including an H429F Fc region mutation did not affect FcγRIII activation by opsonised Ramos-S cells (FIG. 10). In contrast, the inclusion of the mutated H429Y Fc region in of all ACE2-Fc fusion proteins ablated FcγRIIIa activation of cells, which was consistent with the abovementioned near complete failure to bind to Fc receptors, especially FcγRIIIa (FIG. 9B). Thus, while enhancing virus neutralisation, the H429Y modified Fc region in the trACE2-Fe, flACE2-Fc and EflACE2-Fc fusion proteins were largely inactive in FcγR binding (FIG. 9) and consequently incapable of activating cells through FcγRIIIa (FIG. 10). The hierarchy of FcγRIIIa activation by the fusion proteins was trACE2-Fc-kif>EflACE2-Fc-H429F˜EflACE2-Fc-WT˜flACE2-Fc-H429F˜flACE2-Fc-WT>trACE2-Fc-H429F˜trACE2-Fc-WT.

### Complement Activation and Lysis of Spike Cells by ACE2 Fc Proteins

The ACE2-Fc fusion proteins comprising mutated Fe region components were examined for their capacity to fix complement components C1q and C5-C9 in an ELISA-based analysis using streptavidin immobilised RBD-biotin (FIG. 11A-F), and importantly to mediate complement-dependent killing of cells expressing SARS-CoV-2 spike protein (FIG. 11G). In the cell-free ELISA analysis, when the RBD was limiting (FIG. 11A, 111B), or the ACE2-Fc fusion protein was limiting (FIG. 11C-F), the activity of the fusion proteins comprising a mutated H429F Fc region was greatly enhanced over the counterparts comprising a wild type Fc region. Additionally, the formation of C5b-9 (FIG. 11E, 11F) which forms the membrane attack complex on cells, was equivalent for the fusion proteins comprising a mutated H429F Fc region. Further, despite the oligomerisation of the Fc-H429Y fusion proteins (FIGS. 2 and 6), a feature associated with their superior CoV-2 neutralisation activities (FIG. 7), both the trACE2-Fc-H429Y and flACE2-Fc-H429Y fusion proteins showed near equivalent or less C1q or C5-9 fixation compared to the counterparts comprising a wild type Fc region (FIG. 11A-F). Moreover, it was apparent that the trACE2-Fc fusion proteins were more potent in fixing complement C1q and C5b-9 than their flACE2-Fc counterparts (cf FIG. 11C, 11E with 11D, 11F); possibly due to the collectrin dimerisation domain of flACE2-Fe reducing the segmental flexibility of the fusion protein and thereby adversely affecting complement C1q binding. Notably however, this inferior C1q fixing activity of the flACE2-Fc format was overcome by the inclusion of the H429F Fc mutation in the flACE2-Fc-H429F (FIG. 11D) thus conferring C1q binding activity though less than that of the trACE2-Fc H429F fusion protein (FIG. 11C). The inferior activity of the flACE2-Fc format was particularly evident in the weak formation of the C5b-C9 complex by the H429Y Fc variant flACE2-Fc proteins (Yₒₗᵢ and Yₘₙ; FIG. 11F) compared to their trACE2-Fc-H429F counterparts (FIG. 11E).

Surprisingly, the fusion proteins comprising a mutated H429F Fe region, were the only highly active ACE2-Fc fusion proteins in serum complement-dependent cytotoxicity (CDC) of Ramos-S cells (FIG. 11G). In contrast, and despite the capacity of the trACE2-Fc-WT, flACE2-Fc-WT and EflACE2-Fc-WT fusion proteins, as well as the trACE2-Fe-kif fusion protein, to bind and fix C1q and C5b-9 in the ELISA assay (FIG. 11A, 11C, 11E), these fusion proteins could not induce complement-mediated cell death. The trACE2-Fc-H429Yₘₙ fusion protein was also weakly active in complement-mediated killing.

Since it is well known that the activation of the complement cascade can also lead to, for example, the phagocytosis of cells, microbes or particles opsonised with C1q (or other complement fragments such as, for example C3b or C3bi; Ricklin D et al., Immunol Rev 274(1); 33-58, 2016) which bind to specific cell surface receptors such as, for example, CR1 or CR3 (Vandendriessche S et al., Front Cell Dev Biol 9:624025, 2021) on phagocytic cells, it is considered that the observed enhancement of Fc-dependent complement lysis by the ACE2-Fc fusion proteins including an H429F mutation, will also be reflected in similar enhancements in complement-dependent phagocytosis of ACE2-Fc-coated targets (e.g. virions) through the complement receptors.

ACE2-Fc Fusion Protein Interaction with SARS-CoV-2 Variants and Related Coronaviruses

Lastly, the binding of biotinylated trACE2-Fc to RBD-WT and an array of 24 CoV-2 RBD variants was inhibited by the unlabelled decoys, trACE2-Fc (average IC₅₀ across RBD variants ±SEM; 111±3 nM), and with increasing effectiveness by flACE2-Fc (average IC₅₀=86±18 nM, p<0.0001 repeated measure one way ANOVA versus trACE2-Fc) and EflACE2-Fc (average IC₅₀=10±0.4 nM, p<0.0001) (FIG. 12). Notably, effective inhibition of trACE2-Fc binding to all individual RBD variants was observed, with IC₅₀ values within two-fold of that observed for the RBD-WT, including the S477N, E484K and other variants associated with escape from neutralising antibodies (Liu Z et al., Cell Host Microbe Epub 2021/02/04. doi: 10.1016/j.chom.2021.01.014, 2021). The fusion proteins were also effective inhibitors of binding to the spike proteins of the SARS CoV and NL63 viruses. In contrast, the inhibitory mAb S35 was ineffective in preventing trACE2-Fc binding to the CoV-2 RBD variants L455F and A475V and to the spike proteins of SARS-CoV-1 or CoV-NL63. These data indicate the universal nature of the interaction of the ACE2-Fc fusion proteins with the SARS-CoV-2 spike protein RBD and variants, as well as with the spike proteins of those related viruses that also utilise the ACE2 cell entry pathway, and underscores their potential utility for biosecurity against all coronaviruses using ACE2-mediated entry.

### CONCLUSION

It has been found that immunotherapeutic proteins comprising an angiotensin converting enzyme 2 (ACE2) polypeptide (or a fragment thereof) linked to a polypeptide comprising an Fc region component provide considerable potential for the treatment or prevention of coronavirus infection and may also be an effective antiviral agent against related, newly emergent virus species and strains. Selection of, for example, a full-length or truncated (i.e. fragment) of ACE2, and various modifications of the Fc component may also enable considerable “tuning” of the antiviral agent to modify the action(s) by which the antiviral effect is achieved. For example, by including an Fc component in the immunotherapeutic protein comprising an amino acid substitution at the position corresponding to H429 of the amino acid sequence of the human IgG1 heavy chain polypeptide (EU numbering), preferably an H429Y mutation, an oligomeric immunotherapeutic protein may be produced which shows increased virus neutralisation, and abrogated FcγR binding and activation. On the other hand, by including an Fc component in the immunotherapeutic protein comprising an H429F mutation, an antiviral effect including complement-dependent cytotoxicity (CDC) of spike protein-expressing cells (e.g. infected cells) and virions may be achieved. Further, the effect of mutations in the Fc component on the antiviral effect of the immunotherapeutic protein was found to be influenced by the particular ACE2 polypeptide (or fragment thereof) included in the immunotherapeutic protein; as the inclusion of the H429Y mutation in the Fc component enhanced virus neutralisation when the immunotherapeutic protein comprised a full-length (fl) ACE2 ectodomain fragment (i.e. wherein the fragment included the collectrin domain), while the inclusion of the H429F mutation in the Fe component enhanced complement activation most when the immunotherapeutic protein comprised a truncated (tr) ACE2 ectodomain fragment (i.e. wherein the fragment does not include the collectrin domain). Thus, these unique Fe mutations enable complementary approaches to tuning the function of the Fc region component that may aid in the development of ACE2-Fc fusion proteins by allowing the selection of desired functional profiles.

### Example 2 ACE-2-Fc (with H429F) in Combination with an Anti-SARS-CoV-2 Antibody

The effect of H429 modification on the potency of CDC killing in the context of infectious disease was evaluated using mAbs detecting the spike protein of SARS-CoV-2. In particular, the histidine 429 of CH3 was substituted with phenylalanine (i.e. H429F) in the H chain of the S2P6 monoclonal antibody which targets the S2 stem region of the SARS-CoV-2 spike (see, for example, Pinto D et al., Science 373, 1109-1116, 2021).

### Methods and Materials

### Generation of Antibodies and Antibody Constructs

The S2P6-wild type (WT) mAb used in this example comprised a SARS-CoV-2 spike protein-specific heavy chain polypeptide with the amino acid sequence shown as SEQ ID NO: 7 (see Table 4) comprising, in the order of the N-terminus to the C-terminus, the spike-specific VH domain of S2P6 mAb (defined in Pinto et al., 2021 supra) fused to CH1-hinge-CH2-CH3 domains of human IgG1, and is encoded by the codon-optimised polynucleotide sequence shown as SEQ ID NO: 8 (Table 4). The polypeptide of the anti-S2P6 anti-SARS-CoV-2 light chain comprised the amino acid sequence shown as SEQ ID NO: 9 (comprising the spike-specific VL domain of the S2P6 mAb fused to human kappa constant domain) and is encoded by the codon-optimised polynucleotide sequence shown as SEQ ID NO: 10 (Table 4).

Two additional mAbs were generated, the CC40.8-WT mAb and the CV3-25-WT mAb, and used in this example. Each mAb comprised a SARS-CoV-2 spike protein-specific heavy chain polypeptide with the amino acid sequence for either CC40.8 (shown as SEQ ID NO: 11) or for CV3-25 (shown as SEQ ID NO: 12) comprising, in the order of the N-terminus to the C-terminus, the spike-specific VH domain of either CC40.8 mAb (defined in Zhou P et al., Sci Transl Med 14(637):eabi9215, 2022) or of CV3-25 (defined in Jennewein M F et al., Cell Rep 36(2):109353, 2021) fused to CH1-hinge-CH2-CH3 domains of human IgG1, and encoded by the codon-optimised polynucleotide sequence shown for CC40.8 as SEQ ID NO: 13, or for CV3-25 as SEQ ID NO: 14 (see Table 4 for the sequences). The polypeptide of the anti-spike CC40.8 light chain or of the anti-spike CV3-25 light chain comprised the amino acid sequence shown in Table 4 for CC40.4 as SEQ ID NO: 15 and for CV3-25, shown as SEQ ID NO: 16 (comprising the spike-specific VL domain of the CC40.8 mAb or of CV3-25 fused to human kappa constant domain) and is encoded by the codon-optimised polynucleotide sequence shown for CC40.8 as SEQ ID NO: 17 and for CV3-25 as the codon-optimised DNA sequence shown as SEQ ID NO: 18.

### Synthesis of Unmodified and Mutated Heavy Chains

Antibody expression vectors which included a modified Fc sequence encoding the substitution of H429 with phenylalanine (i.e. H429F) in the H chains of the S2P6, CC40.8 and CV3-25 mAbs, were generated by standard methodologies known to those skilled in the art.

Expression and Purification of flACE2-Fc Fusion Proteins.

The flACE2-Fc-WT and flACE2-Fc-H429F fusion proteins were produced using transient transfection in Expi293F cells as described above in Example 1. Purification from supernatant by ion exchange chromatography and size exclusion chromatography (under the same conditions described in Example 1) yielded a single fusion protein “species”.

### Expression of Anti-SARS-CoV-2 Antibody Constructs

Expression of the anti-SARS-CoV-2 spike antibodies was conducted using transient transfection of Expi293F cells (Thermo Fisher Scientific) using standard methodologies. The cell cultures were harvested after transfection and centrifuged at 2500 rpm for 20-30 minutes, and the supernatant filtered with a 0.2 μm high flow filter (Sartorius AG, Göttingen, Germany) prior to purification. The presence of the expected antibody in the supernatant was confirmed by SDS-PAGE.

Purification of mAbs and Evaluation of Antigen Binding

The anti-SARS-CoV-2 mAbs were purified from supernatant of the transfected Expi293F cells by protein A affinity chromatography then size exclusion chromatography (SEC) yielding the expected single IgG peak (i.e. for the standard H₂L₂ antibody structure) confirmed by SDS-PAGE. Prior to functional analysis, the purified flACE-2-Fc-H429F or flACE2-Fc-WT fusion proteins and the purified anti-SARS-CoV-2 mAbs were tested for antigen binding on Ramos cells expressing SARS-CoV-2 spike (Ramos-S cells) by flow cytometry using an FITC-conjugated anti-hIgG-Fc secondary reagent (1/500 dilution; Chemicon®, Merck KGaA, Darmstadt, Germany).

### Complement Dependent Lysis of Cells

The potency of CDC mediated by the flACE2-Fc fusion protein or the anti-SARS-CoV-2 mAbs was measured by flow cytometry using Ramos-S cells expressing the SARS-CoV-2 spike protein in a similar manner to the CDC assay described in Example 1. Human serum, diluted 1/6.4, was used as a source of complement. The Zombie Green Fixable Viability kit (BioLegend) was used following opsonisation of Ramos-S cells with the mAbs. Additionally, CDC lysis was examined in mixtures of flACE2-H429F and anti-SARS-CoV-2 mAbs comprising H chains with an H429F modification.

### Results and Discussion

H429 Modification Confers CDC by the flACE2-Fc Fusion Protein

The flACE2-Fc-H429F and flACE2-Fc-WT fusion proteins exhibited near identical binding characteristics to SARS-CoV-2 spike protein expressed on Ramos-S cells (FIG. 13A) indicating that the H429F mutation did not appreciably affect antigen binding on cells. The flACE2-Fc-H429F and flACE2-Fc-WT fusion proteins were then tested for their capacity to mediate CDC (FIG. 13B). Despite their similar binding profiles, considerable differences in the CDC potency were observed between the two fusion proteins. That is, flACE2-Fc-WT showed poor CDC of Ramos-S cells, with the maximum kill of 19.4% occurring at the highest concentration (2.5 Ng/ml flACE2-Fc-WT) and rapid titration to background (2.5 μg/ml-0.6 μg/ml). In contrast, the flACE2-Fc-H429F fusion protein mediated a potent level of cell killing over a larger concentration range (2.5 μg/ml-0.156 μg/ml) compared to flACE2-Fc-WT, and exhibited a maximum kill of 73.8%.

H429 Modification Confers CDC by Anti-SARS-CoV-2 mAbs

The anti-SARS-CoV-2 mAbs S2P6-WT, CC40.8-WT and CV-23-WT were formatted as wild type human IgG1 and kappa light chain mAbs as described above. The H429F mutation was engineered into the H chains of each mAb to generate S2P6-H429F, CC40.8—H429F and CV3-25-H429F mAbs. The binding of the purified mAbs to SARS-CoV-2 spike protein was evaluated by flow cytometry (FIG. 13C). The S2P6-H429F mAb and S2P6-WT mAb showed near identical binding properties. Similarly, the CC40.8—H429F and CV3-25-H429F mAbs showed similar binding profiles to their respective wild type counterparts, indicating that the H429F modification of the heavy chain does not affect interaction of the mAbs with the SARS-CoV-2 spike antigen.

The CDC potency of the S2P6-H429F and CC40.8—H429F mAbs, relative to their wild type counterparts S2P6-WT and CC40.8-WT, was also determined (see FIG. 13D). It was found that the S2P6-WT mAb showed no CDC of Ramos-S cells at any concentration above the background control of lysis in the absence of antibody but in the presence of only complement (i.e. no mAb C′ only; FIG. 13D). However, the S2P6-H429F mAb showed potent killing. Similarly, the CC40.8—H429F mAb also showed readily detectable killing above background though not as great as the potency of S2P6-H429F.

Modification of the CH3 Domain Enables Fusion Proteins and mAbs of Distinct Specificity to Act Synergistically in CDC

Combinations of the ACE2-Fc fusion protein and anti-SARS-CoV-2 mAbs were investigated for potential synergy leading to greater functional potency (i.e. by determination of enhanced CDC lysis of targets) (see FIGS. 14A and 14B). In particular, cooperation in CDC between the fusion protein (which binds with the RBD of the spike protein) and anti-SARS-CoV-2 mAbs (which detect epitopes in the stem region of the spike protein), was determined using pairwise combinations of flACE2-Fc with the various anti-SARS-CoV-2 mAbs.

The combination of flACE2-Fc-WT with the S2P6-WT mAb failed to show any CDC above background. Moreover, no lysis was detected when the S2P6-WT was titrated in the presence of a fixed concentration (1 μg/ml) of flACE2-Fc-WT (S2P6-WT+flACE2-Fc-WT; FIG. 14A). In contrast, functional cooperativity was readily apparent between the flACE2-Fc-H429F fusion protein and the S2P6-H429F mAb (FIG. 14A). The S2P6-H429F mAb, when titrated alone, mediated potent CDC of Ramos-S cells in the absence of flACE2-Fc-H429F. The flACE2-Fc-H429F at the limiting concentration of 1 μg/ml, mediated detectable CDC (23.43% killing). Remarkably, the level of CDC mediated at all concentrations of S2P6-H429F in the presence of a fixed concentration (1 μg/ml) of flACE2-Fc-H429F, was considerably greater than with the S2P6-H429F mAb alone or of the flACE2-Fc-H429F alone (FIG. 14A). The cooperation between the flACE2-Fc-H429F fusion protein and the S2P6-H429F mAb was especially evident when the concentration of the mAb was limiting (0.03 μg/ml-0.125 μg/ml) shown by the arrows in FIG. 14A. The cooperation was also evaluated between flACE2-Fc-H429F and two additional anti-SARS-CoV-2 spike mAbs, namely CC40.8 and CV3-25 (FIG. 14B). CDC was determined for the mAbs alone (at 2.5 μg/ml of mAb), flACE2-Fc-H429F alone and in pairwise mixtures. Increased CDC was evident in the combination of the flACE2-Fc-H429F fusion protein and the CC40.8—H429F mAb compared to the CDC mediated by either alone. Similarly, the combination of flACE2-Fc-H429F with the CV3-25-H429F mAb mediated an increased level of CDC compared to the killing by either alone (FIG. 14B). Clearly, the surprising cooperation between the fusion protein and monoclonal antibodies can occur broadly and is not restricted to a single combination of proteins. More remarkable is that such cooperation was achieved between two proteins of distinct molecular formats (i.e. a fusion protein comprising a receptor (ACE2) fused to an Fc region component with a H429F modification synergistically cooperating with a monoclonal antibody to achieve greater functional potency).

### Example 3 Production of an Ab-Like Molecule from an ACE2-Fc Fusion Protein

In this example, an immunotherapeutic ACE2-Fc fusion protein comprising an Fc region component with an amino acid substitution at position H429 of the amino acid sequence of human IgG1 heavy chain, was produced having a structure like the standard H₂L₂ antibody structure. This immunotherapeutic protein is an example of an antibody-like (Ab-like) molecule; more particularly, an example of an Ab-like fusion protein. Molecules of this type are depicted in FIG. 15A. As shown such molecules has the H₂L2 format of an antibody but have one or more cell surface receptor polypeptide/co-receptor polypeptide (or fragment thereof) instead of the typical VL or VH domain of immunoglobulins.

### Methods and Materials

### Constructs

Expression constructs were designed and assembled using NEBuilder (New England Biolabs) for the expression of Ab-like molecules based upon the EflACE2-Fc-WT and EflACE2-Fc-H429F fusion proteins described in Example 1. The mature EflACE2-Ab-like-WT fusion protein comprised a heavy (H) chain fusion protein shown as SEQ ID NO: 19 (Table 4) comprising the entire EflACE2 ectodomain fused, in the order of the N-terminus to the C-terminus, to the CH1-hinge-CH2-CH3 domains of wild type human IgG1 (as encoded by the codon-optimised polynucleotide sequence shown as SEQ ID NO: 20), and a light (L) chain fusion protein shown as SEQ ID NO: 21 comprising the entire EflACE2 ectodomain fused to the N-terminus of a human kappa constant domain (as encoded by the codon-optimised polynucleotide sequence shown as SEQ ID NO: 22). The mature EflACE2-Ab-like-H429F fusion protein comprised a heavy (H) chain fusion protein shown as SEQ ID NO: 23 with an Fc region component including the H429F modification (as encoded by the codon-optimised polynucleotide sequence shown as SEQ ID NO: 24) and the same light (L) chain fusion protein as the EflACE2-Ab-like-WT fusion protein. Thus, the expression constructs consisted of several synthetic polynucleotide sequences that, joined together, encoded the heavy chains or light chain, within the expression plasmid pcDNA3.4 (Thermo Fisher Scientific).

### Expression and Purification

Expression of the Ab-like fusion proteins was conducted using transient transfection of Expi293F cells (Thermo Fisher Scientific) using the conditions described hereinbefore in Example 1, following which, the transfected Expi293F cells were incubated at 37° C., and then after the addition of Enhancers (Thermo Fisher Scientific), the transfected Expi293F cells were then incubated at 34° C., 125 rpm, and 8% CO₂ atmosphere for a further six days. For purification, the supernatant was first harvested by centrifugation at 3000×g for 15 minutes, and then the supernatant collected into a fresh tube and spun again for an additional 30 minutes. The supernatant was then filtered through a 0.22 μm filter. The EflACE2-Ab-like fusion proteins were purified using a 1 mL Hitrap™ Protein A column (Cytiva Life Sciences, Marlborough, MA, United States of America) equilibrated with 20 mM phosphate buffer, pH 7.0, with all steps at a flowrate of 1 ml/min. The filtered supernatant was then loaded onto the Protein A column using a BioLogic LP low-pressure chromatography system (Bio-Rad, Hercules, CA, United States of America). After loading was completed, the column was washed with 20 Column Volumes (CV) of 20 mM phosphate buffer pH 7.0. After further washing with 5CV of Buffer A—30 mM arginine pH 4.0, the bound fusion proteins were eluted using a shallow linear gradient to 35% buffer B—130 mM arginine (pH 4.0) performed over 20 CV. Fractions of 1 mL were collected, neutralised using 1M Tris-HCl pH 9.0, and concentrations determined using the 0.1% (mg/ml) extinction coefficient at 280 nm of 1.80 (derived using protparam at https://web.expasy.org/protparam/). Fractions 15, 20, 24, 25 and 27 across the elution peak were analysed using size exclusion chromatography (SEC). Because of the gentle elution conditions of this shallow arginine gradient, all of these tested fractions (i.e. 15, 20, 24, 25 and 27) showed approximately the same monomer to oligomer ratio of ˜85:15% and were free of large molecular weight aggregates. Eluted fractions containing the fusion protein (for the EFIACE2-Ab-like-H429F fusion protein, this was fractions 8-34; see FIG. 16A) were pooled and concentrated using a 30 kDa cut-off filtration device (Merck) and separated by SEC using a Superose 6 Increase 10/300 GL column (Cytiva Life Sciences). The pooled and concentrated fusion proteins eluted from the Protein A column had a similar SEC profile (shown for EF1ACE2-Ab-like-H429F in FIG. 16B) to that of the individual fractions 15, 20, 24, 25 and 27 (data not shown). SDS-PAGE analysis indicated, for both of the Ab-like fusion proteins, that both the Protein A pooled eluate and the SEC-derived monomeric fraction comprised a heavy (H) and light (L) chain on reduction, and under non-reducing conditions comprised a fully disulphide-bonded, high molecular weight species consistent with a H₂L₂ format (shown for EFlACE2-Ab-like-H429F in FIG. 16C).

### Complement Dependent Cytotoxicity (CDC) Assay

CDC potency was evaluated by flow cytometry using the Zombie Green method in a similar manner to the CDC assay described in Example 1. Binding of the EflACE2-Ab-like proteins to SARS-CoV-2 spike antigen was evaluated by flow-cytometry using Ramos-S cells as described herein in Example 1.

### Results and Discussion

In this example, the affinity enhanced full length ACE2 polypeptide (EflACE2) was used in the production of EflACE2-Ab-like fusion protein comprising a self-assembled polypeptide complex with an H₂L₂ format like that of an antibody, That is, the expressed Ab-like fusion proteins had two heavy (H) chain-like polypeptides each comprising an EflACE2 polypeptide linked to an Fc region component (i.e. the H chain) and additionally, two light (L) chain-like polypeptides each comprising an EflACE2 polypeptide linked to a constant (C) domain of a L chain.

### Production and Purification of the Ab-Like Fusion Proteins

As described above in Example 1, ACE2-Fc fusion proteins can be appropriately purified by sequential ion exchange (IEX) chromatography and size exclusion chromatography. However, in this example, for the purification of the Ab-like fusion proteins, instead of the IEX step, Protein A affinity chromatography (which is a highly specific affinity purification and is appropriate for at-scale production in an industrial setting), was used with arginine in the conditions for protein elution to suppress aggregate formation (see, for example, Arakawa T et al., Protein Expr Purif 36(2):244-248, 2004; 2004 et al., Ejima D et al., Anal Biochem 345(2):250-257, 2005; and Shukla D et al., J Phys Chem B 115(11):2645-2654, 2010).

In particular, the purification of the Ab-like fusion proteins were eluted using a low concentration of arginine, less than 130 mM, and at less than or equal to pH 5. This low arginine concentration can be achieved by, for example, standard liquid chromatography systems that can deliver a buffer gradient to the column. For example, for EflACE2-Ab-like-H429F, the purification was achieved through binding to the Protein A column under standard conditions followed by washing with five column volumes of Buffer A—30 mM arginine (pH 4.0), and elution thereafter with a linear gradient to 35% of 130 mM arginine (pH 4.0); yielding the Ab-like fusion protein in a highly pure form (as determined by SDS-PAGE) which was also largely monomeric (˜85%) as determined by SEC (i.e. relatively free of aggregates)(see FIGS. 15B and 16C; which show that the two Ab-like fusion proteins were obtained as a pure, fully disulphide-bonded H₂L₂ protein that, on reduction with dithiothreitol, generated equal amounts of EflACE2-H and EflACE2-L chains). In contrast, it was found that elution under other conditions (e.g. 100 mM sodium citrate buffer pH 3.0) may result in the elution of the ACE2-Ab-like fusion proteins (or even of the ACE2-Fc fusion proteins of Example 1) with some aggregates.

### Target Recognition and CDC Potency of Ab-Like Fusion Protein.

The CDC potency of the EflACE2-Ab-like-WT and EflACE2-Ab-like-H429F fusion proteins was determined by flow cytometry analysis (FIG. 15C). The EflACE2-Ab-like-WT failed to mediate detectable CDC killing of Ramos-S cells; indeed, the level of lysis was similar to the background lysis (5.1%) of the cells in the complement only control. In contrast, the EflACE2-Ab-like-H429F fusion protein showed that it mediated powerful CDC killing of the SARS-CoV-2 spike expressing Ramos-S cells. Interestingly, these differences in CDC potency were not attributable to differences in binding of targets by the WT and H429F forms of the Ab-like proteins since analysis by flow cytometry, of the binding of the respective proteins to Ramos-S cells expressing SARS-CoV2 spike protein, demonstrated that the EflACE2-Ab-like-WT and the EflACE2-Ab-like-H429F proteins showed comparable levels of binding to the cells (FIG. 15D).

Immunotherapeutic ACE2-Fc fusion proteins were successfully produced having a structure like the standard H₂L₂ antibody structure. Where these Ab-like fusion proteins included an H429 modification of the heavy (H) chain, an enhanced level of CDC function was achieved. While each of the heavy chain and light chain fusion proteins used to produce the Ab-like proteins comprised an ACE2 ectodomain as the cell surface receptor polypeptide/co-receptor polypeptide (or fragment thereof), it is to be appreciated that similar Ab-like fusion proteins may be produced wherein more than one cell surface receptor polypeptide/co-receptor polypeptide (or fragment thereof) is represented. For instance, as shown in FIG. 15A, right panel, the Ab-like fusion protein may comprise a total of four different cell surface receptor polypeptide/co-receptor polypeptide (or fragment thereof) (i.e. wherein each of X1, X2, X3 and X4 in the depicted molecule are different). The different cell surface receptor polypeptide/co-receptor polypeptide (or fragment thereof) may be targeted against the same virus or against different viruses or viral stains (e.g. to provide a Ab-like protein for use as tetravalent antiviral agent).

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the immunotherapeutic proteins, uses and pharmaceutical composition of the present disclosure are not restricted by the particular application(s) described. Neither are the immunotherapeutic proteins, uses and pharmaceutical composition restricted in their preferred embodiment(s) with regard to the particular elements and/or features described or depicted herein. It will also be appreciated that the immunotherapeutic proteins, uses and pharmaceutical composition of the present disclosure are not limited to the embodiment or embodiments disclosed, but are capable of numerous rearrangements, modifications and substitutions without departing from the scope of the disclosure as set forth and defined by the following claims.

TABLE 4
SEQUENCES

SEQ ID NO: 7
S2P6 IgG1 WT H chain amino acid sequence
EVQLVQSGAEVKKPGASVKVSCKASGYTFTSQYMHWVRQAPGQGLEWIGIINPSGVHTSYAQKFQGRVTLTRDTST
STLYMELSSLRSEDTAVYYCARGSPKGAFDYWGQGTLVTVSSASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSREEMTKNQVSLTCLVKGFYPSDIAV
EWESNGQPENNYKTTPPVLDSDGSFFLYSKLTVDKSRWQQGNVFSCSVMHEALHNHYTQKSLSLSPGK
SEQ ID NO: 8
S2P6 IgG1 WT H chain nucleotide sequence
GAAGTTCAGCTGGTTCAGTCTGGCGCCGAAGTGAAGAAACCTGGCGCCTCTGTGAAGGTGTCCTGCAAGGCCAGCG
GCTACACCTTTACCAGCCAGTACATGCACTGGGTCCGACAGGCTCCAGGACAGGGACTCGAGTGGATCGGCATCAT
CAATCCTAGCGGCGTGCACACCAGCTACGCCCAGAAATTCCAGGGCAGAGTGACCCTGACCAGAGACACCAGCACC
AGCACACTGTACATGGAACTGAGCAGCCTGCGGAGCGAGGATACCGCCGTGTACTATTGTGCCAGAGGCTCTCCTA
AGGGCGCCTTCGATTATTGGGGCCAGGGCACACTGGTCACCGTGTCCTCTGCTAGCACAAAAGGCCCTAGCGTCTT
TCCACTCGCCCCATCCTCCAAGAGCACCTCTGGGGGCACTGCGGCTTTGGGCTGCTTGGTGAAGGATTACTTCCCA
GAGCCTGTCACTGTCAGCTGGAATAGCGGGGCTCTGACCAGTGGAGTGCACACCTTTCCCGCCGTGTTACAGAGCA
GCGGCCTCTATAGCCTGAGCAGTGTGGTAACTGTGCCCTCGAGCAGCTTGGGTACCCAGACCTATATCTGCAATGT
CAATCATAAACCCAGTAACACAAAGGTAGACAAGAAAGTCGAACCCAAATCTTGCGACAAAACTCACACATGCCCT
CCTTGCCCCGCCCCCGAACTCCTGGGGGGACCTTCGGTTTTTCTTTTTCCACCTAAACCGAAAGACACCCTGATGA
TCAGCAGGACACCAGAAGTGACATGTGTCGTCGTGGACGTATCCCATGAGGACCCGGAGGTGAAGTTCAACTGGTA
TGTTGATGGTGTGGAAGTGCATAATGCAAAAACTAAGCCACGGGAGGAACAGTACAATAGCACATACAGAGTAGTT
AGCGTTCTGACTGTCCTGCACCAAGATTGGCTGAACGGGAAGGAATACAAGTGTAAGGTGAGCAACAAAGCCCTGC
CCGCTCCAATTGAGAAAACAATTTCTAAGGCCAAAGGACAGCCCCGGGAGCCACAGGTGTATACTCTGCCGCCTAG
CAGGGAGGAAATGACCAAGAACCAGGTGAGCCTGACCTGTCTGGTGAAAGGCTTCTACCCCAGCGATATCGCAGTT
GAGTGGGAGAGCAATGGCCAACCCGAGAACAACTACAAGACTACGCCCCCCGTGCTTGATAGCGACGGATCATTTT
TCCTGTACTCAAAACTGACCGTGGACAAAAGCAGATGGCAGCAGGGAAACGTTTTCAGTTGCTCAGTGATGCACGA
AGCGCTGCACAATCATTATACTCAGAAAAGCCTGAGCTTGAGCCCGGGAAAA
SEQ ID NO: 9
S2P6 kappa light chain amino acid sequence
EIVMMQSPGTLSLSPGERATLSCRASQSVRSNYLAWYQQKPGQAPRLLIYGASSRATGIPDRFSGSGSGTDETLTI
SRLEPEDFAVYYCQQYGSSPPRFTFGPGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVTKSFNRGEC
SEQ ID NO: 10
S2P6 kappa light chain nucleotide sequence
GAGATCGTCATGATGCAGAGCCCCGGCACACTGTCACTGTCTCCAGGCGAAAGAGCCACACTGAGCTGTAGAGCCT
CTCAGAGCGTGCGGAGCAATTACCTGGCCTGGTATCAGCAGAAGCCCGGACAGGCTCCTCGGCTGTTGATCTATGG
CGCCTCTAGCAGAGCCACTGGCATCCCCGATAGATTTTCTGGCAGCGGCTCCGGCACCGACTTCACCCTGACAATC
AGCAGACTGGAACCCGAGGACTTCGCCGTGTACTACTGTCAGCAGTACGGCAGCAGCCCTCCTCGGTTCACATTTG
GCCCTGGTACCAAGGTGGAAATCAAGCGGACAGTGGCCGCTCCTAGCGTGTTCATCTTTCCACCTAGCGACGAGCA
GCTGAAGTCTGGCACAGCCTCTGTCGTGTGCCTGCTGAACAACTTCTACCCCAGAGAAGCCAAGGTGCAGTGGAAG
GTGGACAACGCCCTGCAGAGCGGCAATAGCCAAGAGAGCGTGACCGAGCAGGACAGCAAGGACTCTACCTACAGCC
TGAGCAGCACCCTGACACTGAGCAAGGCCGACTACGAGAAGCACAAAGTGTACGCCTGCGAAGTGACCCACCAGGG
CCTTTCTAGCCCTGTGACCAAGAGCTTCAACCGGGGCGAGTGC
SEQ ID NO: 11
CC40.8 IgG1 WT H Chain amino acid sequence
QVQEVQLLESGGGLVQPGGSLRLSCAASGFTESSYVMTWARQAPGKGLEWVSAISGTGYTYYADSVKGRFTVSRDN
SKNTLFLQMSSLRAEDTAVYYCAITMAPVVWGOGTTVTVSSASTKGPSVFPLAPSSKSTSGGTAALGCLVKDYFPE
PVTVSWNSGALTSGVHTFPAVLQSSGLYSLSSVVTVPSSSLGTQTYICNVNHKPSNTKVDKKVEPKSCDKTHTCPP
CPAPELLGGPSVFLFPPKPKDTLMISRTPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPPSREEMTKNQVSLTCLVKGFYPSDIAVE
WESNGQPENNYKTTPPVLDSDGSFFLYSKLTVDKSRWQQGNVFSCSVMHEALHNHYTQKSLSLSPGK
SEQ ID NO: 12
CV3-25 WT H chain amino acid sequence
QVQLVESGAEVKKPGESLKISCKGSGYTFTRYWIGWVRQMPGKGLEWMGIIYPGDSDTRYSPSFQGHVTISADKSI
STAYLQWNSLKASDTAMYYCARLPQYCSNGVCQRWFDPWGQGTLVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVMHEALHNHYTQKSLSLSPGK
SEQ ID NO: 13
CC40.8 IgG1 WT H chain nucleotide sequence
CAAGTGCAGGAAGTGCAGCTGCTGGAATCTGGCGGAGGACTGGTTCAACCAGGCGGCTCTCTGAGACTGTCTTGTG
CCGCCAGCGGCTTCACCTTCAGCAGCTACGTTATGACCTGGGCCAGACAGGCCCCTGGCAAAGGACTTGAATGGGT
GTCCGCCATCAGCGGCACAGGCTACACCTACTACGCCGACTCTGTGAAGGGCAGATTCACCGTGTCCAGAGACAAC
AGCAAGAATACCCTGTTCCTGCAGATGAGCAGCCTGAGAGCCGAGGATACCGCCGTGTACTACTGCGCCATTACAA
TGGCCCCTGTCGTGTGGGGCCAGGGCACAACAGTGACAGTCAGCAGCGCTAGCACAAAAGGCCCTAGCGTCTTTCC
ACTCGCCCCATCCTCCAAGAGCACCTCTGGGGGCACTGCGGCTTTGGGCTGCTTGGTGAAGGATTACTTCCCAGAG
CCTGTCACTGTCAGCTGGAATAGCGGGGCTCTGACCAGTGGAGTGCACACCTTTCCCGCCGTGTTACAGAGCAGCG
GCCTCTATAGCCTGAGCAGTGTGGTAACTGTGCCCTCGAGCAGCTTGGGTACCCAGACCTATATCTGCAATGTCAA
TCATAAACCCAGTAACACAAAGGTAGACAAGAAAGTCGAACCCAAATCTTGCGACAAAACTCACACATGCCCTCCT
TGCCCCGCCCCCGAACTCCTGGGGGGACCTTCGGTTTTTCTTTTTCCACCTAAACCGAAAGACACCCTGATGATCA
GCAGGACACCAGAAGTGACATGTGTCGTCGTGGACGTATCCCATGAGGACCCGGAGGTGAAGTTCAACTGGTATGT
TGATGGTGTGGAAGTGCATAATGCAAAAACTAAGCCACGGGAGGAACAGTACAATAGCACATACAGAGTAGTTAGC
GTTCTGACTGTCCTGCACCAAGATTGGCTGAACGGGAAGGAATACAAGTGTAAGGTGAGCAACAAAGCCCTGCCCG
CTCCAATTGAGAAAACAATTTCTAAGGCCAAAGGACAGCCCCGGGAGCCACAGGTGTATACTCTGCCGCCTAGCAG
GGAGGAAATGACCAAGAACCAGGTGAGCCTGACCTGTCTGGTGAAAGGCTTCTACCCCAGCGATATCGCAGTTGAG
TGGGAGAGCAATGGCCAACCCGAGAACAACTACAAGACTACGCCCCCCGTGCTTGATAGCGACGGATCATTTTTCC
TGTACTCAAAACTGACCGTGGACAAAAGCAGATGGCAGCAGGGAAACGTTTTCAGTTGCTCAGTGATGCACGAAGC
GCTGCACAATCATTATACTCAGAAAAGCCTGAGCTTGAGCCCGGGAAAA
SEQ ID NO: 14
CV3-25 WT H chain nucleotide sequence
CAAGTGCAGCTGGTTGAATCTGGCGCCGAAGTGAAGAAGCCTGGCGAGAGCCTGAAGATCAGCTGCAAAGGCAGCG
GCTACACCTTCACCAGATACTGGATCGGCTGGGTCCGACAGATGCCTGGCAAAGGCCTTGAGTGGATGGGCATCAT
CTACCCCGGCGACAGCGATACCAGATACAGCCCTAGCTTTCAGGGCCACGTGACCATCAGCGCCGACAAGTCTATC
AGCACCGCCTACCTGCAGTGGAACAGCCTGAAGGCCAGCGACACCGCCATGTACTACTGTGCCAGACTGCCCCAGT
ACTGCAGCAATGGCGTTTGCCAGCGTTGGTTCGATCCTTGGGGACAGGGCACACTGGTCACCGTTAGCAGCGCTAG
CACAAAAGGCCCTAGCGTCTTTCCACTCGCCCCATCCTCCAAGAGCACCTCTGGGGGCACTGCGGCTTTGGGCTGC
TTGGTGAAGGATTACTTCCCAGAGCCTGTCACTGTCAGCTGGAATAGCGGGGCTCTGACCAGTGGAGTGCACACCT
TTCCCGCCGTGTTACAGAGCAGCGGCCTCTATAGCCTGAGCAGTGTGGTAACTGTGCCCTCGAGCAGCTTGGGTAC
CCAGACCTATATCTGCAATGTCAATCATAAACCCAGTAACACAAAGGTAGACAAGAAAGTCGAACCCAAATCTTGC
GACAAAACTCACACATGCCCTCCTTGCCCCGCCCCCGAACTCCTGGGGGGACCTTCGGTTTTTCTTTTTCCACCTA
AACCGAAAGACACCCTGATGATCAGCAGGACACCAGAAGTGACATGTGTCGTCGTGGACGTATCCCATGAGGACCC
GGAGGTGAAGTTCAACTGGTATGTTGATGGTGTGGAAGTGCATAATGCAAAAACTAAGCCACGGGAGGAACAGTAC
AATAGCACATACAGAGTAGTTAGCGTTCTGACTGTCCTGCACCAAGATTGGCTGAACGGGAAGGAATACAAGTGTA
AGGTGAGCAACAAAGCCCTGCCCGCTCCAATTGAGAAAACAATTTCTAAGGCCAAAGGACAGCCCCGGGAGCCACA
GGTGTATACTCTGCCGCCTAGCAGGGAGGAAATGACCAAGAACCAGGTGAGCCTGACCTGTCTGGTGAAAGGCTTC
TACCCCAGCGATATCGCAGTTGAGTGGGAGAGCAATGGCCAACCCGAGAACAACTACAAGACTACGCCCCCCGTGC
TTGATAGCGACGGATCATTTTTCCTGTACTCAAAACTGACCGTGGACAAAAGCAGATGGCAGCAGGGAAACGTTTT
CAGTTGCTCAGTGATGCACGAAGCGCTGCACAATCATTATACTCAGAAAAGCCTGAGCTTGAGCCCGGGAAAA
SEQ ID NO: 15 CC40.8 L chain amino acid sequence
SYELTQPPSVSVSPGQTARITCSGDALPKRYAYWYQQKSGQAPILVIYEDKKRPSGIPERLSGSKSGTVATLTISG
AQVEDEADYYCYSTDSSGNHAVFGGGTQLTVLGQPKAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID NO: 16 CV3-25 L chain amino acid sequence
EIVLTQSPSSVSASVGDRVTITCRASQGISSWLAWYQQKPGKAPKLLIYAASSLQSGVPSRFSGSGSGTDFTLTIS
SLQPEDFATYYCQQGNSFPYTFGQGTNLEIKRTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNRGEC
SEQ ID NO: 17 CC40.8 L chain nucleotide sequence
AGCTATGAACTGACACAGCCTCCAAGCGTGTCCGTGTCTCCTGGACAGACCGCCAGAATCACATGTAGCGGAGATG
CCCTGCCTAAGAGATACGCCTACTGGTATCAGCAGAAGTCCGGACAGGCCCCTATCCTGGTCATCTACGAGGACAA
GAAGCGGCCCTCTGGCATCCCTGAGAGACTGAGCGGAAGCAAGTCTGGCACAGTGGCCACACTGACAATCTCTGGC
GCCCAGGTGGAAGATGAGGCCGACTACTACTGCTACAGCACCGACAGCTCTGGCAACCACGCTGTGTTTGGCGGAG
GTACCCAACTGACCGTGCTGGGCCAACCTAAAGCCGCTCCTAGCGTGTTCATCTTTCCACCTAGCGACGAGCAGCT
GAAGTCTGGCACAGCCTCTGTCGTGTGCCTGCTGAACAACTTCTACCCCAGAGAAGCCAAGGTGCAGTGGAAGGTG
GACAACGCCCTGCAGAGCGGCAATAGCCAAGAGAGCGTGACCGAGCAGGACAGCAAGGACTCTACCTACAGCCTGA
GCAGCACCCTGACACTGAGCAAGGCCGACTACGAGAAGCACAAAGTGTACGCCTGCGAAGTGACCCACCAGGGCCT
TTCTAGCCCTGTGACCAAGAGCTTCAACCGGGGCGAGTGC
SEQ ID NO: 18 CV3-25 L chain nucleotide sequence
GAAATTGTGCTGACACAGAGCCCCAGCAGCGTGTCAGCCTCTGTGGGAGACAGAGTGACCATCACCTGTAGAGCCA
GCCAGGGCATCTCTTCTTGGCTGGCCTGGTATCAGCAGAAGCCTGGCAAGGCCCCTAAGCTGCTGATCTATGCCGC
TAGCTCTCTGCAGTCTGGCGTGCCCAGCAGATTTTCTGGCAGCGGCTCTGGCACCGACTTCACCCTGACCATATCT
AGCCTGCAGCCTGAGGACTTCGCCACCTACTATTGCCAGCAGGGCAACAGCTTCCCCTACACCTTTGGCCAGGGTA
CCAACCTGGAAATCAAGCGGACAGTGGCCGCTCCTAGCGTGTTCATCTTTCCACCTAGCGACGAGCAGCTGAAGTC
TGGCACAGCCTCTGTCGTGTGCCTGCTGAACAACTTCTACCCCAGAGAAGCCAAGGTGCAGTGGAAGGTGGACAAC
GCCCTGCAGAGCGGCAATAGCCAAGAGAGCGTGACCGAGCAGGACAGCAAGGACTCTACCTACAGCCTGAGCAGCA
CCCTGACACTGAGCAAGGCCGACTACGAGAAGCACAAAGTGTACGCCTGCGAAGTGACCCACCAGGGCCTTTCTAG
CCCTGTGACCAAGAGCTTCAACCGGGGCGAGTGC
SEQ ID NO: 19
EflACE2-Ab-like-WT H chain amino acid sequence
ETGSTIEEQAKYFLDKFNHEAEDLFYQSSLASWNYNTNITEENVQNMNNAGDKWSAFLKEQSTTAQMYPLQEIQNL
TVKLQLQALQQNGSSVLSEDKSKRINTILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDYSRGQLIEDVEHTFEEIKPLYEHLHAY
VRAKLMNAYPSYISPIGCLPAHLLGDMWGRFWTNLYSLTVPFGQKPNIDVTDAMVDQAWDAQRIFKEAEKFFVSVG
LPNMTQGFWEYSMLTDPGNVQKAVCHPTAWDLGKGDFRILMCTKVTMDDFLTAHHEMGHIQYDMAYAAQPFLLRNG
ANEGFHEAVGEIMSLSAATPKHLKSIGLLSPDFQEDNETEINFLLKQALTIVGTLPFTYMLEKWRWMVEKGEIPKD
QWMKKWWEMKREIVGVVEPVPHDETYCDPASLFHVSNDYSFIRYYTRTLYQFQFQEALCQAAKHEGPLHKCDISNS
TEAGQKLFNMLRLGKSEPWTLALENVVGAKNMNVRPLLNYFEPLFTWLKDQNKNSFVGWSTDWSPYADQSIKVRIS
LKSALGDRAYEWNDNEMYLERSSVAYAMRQYFLKVKNQMILFGEEDVRVANLKPRISFNFFVTAPKNVSDIIPRTE
VEKAIRMSRSRINDAFRLNDNSLEFLGIQPTLGPPNQPPVSGGGGSGGGGSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMHEALHNHYTQKSLSLSPG
K
SEQ ID NO: 20
EflACE2-Ab-like-WT H chain nucleotide sequence
GAAACCGGTTCCACCATTGAGGAACAGGCCAAGTACTTTTTGGACAAGTTTAACCACGAAGCCGAAGACCTGTTCT
ATCAAAGTTCACTTGCTTCTTGGAATTATAACACCAATATTACTGAAGAGAATGTCCAAAACATGaACAACGCCGG
CGACAAGTGGAGCGCCTTCCTGAAAGAGCAGAGCACAACAGCCCAGATGTACCCTCTGCAAGAGATCCAGAACCTG
ACCGTGAAGCTCCAGCTGCAGGCCCTCCAGCAGAATGGAAGCTCTGTGCTGAGCGAGGACAAGAGCAAGCGGCTGA
ACACCATCCTGAATACCATGAGCACCATCTACAGCACCGGCAAAGTGTGCAACCCCGACAATCCCCAAGAGTGCCT
GCTGCTGGAACCCGGCCTGAATGAGATCATGGCCAACAGCCTGGACTACAACGAGAGACTGTGGGCCTGGGAGTCT
TGGAGAAGCGAAGTGGGAAAGCAGCTGCGGCCCCTGTACGAGGAATACGTGGTGCTGAAGAACGAGATGGCCAGAG
CCAACCACTACGAGGACTACGGCGACTATTGGAGAGGCGACTACGAAGTGAATGGCGTGGACGGCTACGACTACAG
CAGAGGCCAGCTGATCGAGGACGTGGAACACACCTTCGAGGAAATCAAGCCTCTGTACGAGCATCTGCACGCCTAC
GTGCGGGCCAAGCTGATGAATGCTTACCCCAGCTACATCAGCCCCATCGGCTGTCTGCCTGCTCATCTGCTGGGAG
ACATGTGGGGCAGATTCTGGACCAACCTGTACAGCCTGACAGTGCCCTTCGGCCAGAAACCTAACATCGACGTGAC
CGACGCTATGGTGGATCAGGCTTGGGATGCCCAGCGGATCTTCAAAGAGGCCGAGAAGTTCTTCGTGTCCGTGGGC
CTGCCTAATATGACCCAAGGCTTCTGGGAGTACTCCATGCTGACAGACCCCGGCAACGTGCAGAAAGCCGTGTGTC
ATCCTACCGCCTGGGATCTCGGCAAGGGCGACTTCAGAATCCTGATGTGCACCAAAGTGACGATGGACGACTTCCT
GACAGCCCACCACGAGATGGGCCACATCCAGTACGATATGGCCTACGCCGCTCAGCCCTTCCTGCTGAGAAATGGC
GCCAATGAGGGCTTCCATGAGGCCGTGGGAGAGATCATGAGCCTGTCTGCCGCCACACCTAAGCACCTGAAGTCTA
TCGGACTGCTGAGCCCCGACTTCCAAGAGGACAACGAGACAGAGATCAACTTCCTGCTCAAGCAGGCCCTGACCAT
CGTGGGCACACTGCCCTTTACCTACATGCTGGAAAAGTGGCGGTGGATGGTCTTTAAGGGCGAGATCCCCAAGGAC
CAGTGGATGAAGAAATGGTGGGAGATGAAGCGCGAGATCGTGGGCGTTGTGGAACCTGTGCCTCACGACGAGACAT
ACTGCGATCCTGCCAGCCTGTTTCACGTGTCCAACGACTACTCCTTCATCCGGTACTACACCCGGACACTGTACCA
GTTCCAGTTTCAAGAGGCTCTGTGCCAGGCCGCCAAGCACGAAGGACCTCTGCACAAGTGCGACATCAGCAACTCT
ACAGAGGCCGGACAGAAACTGTTCAACATGCTGCGGCTGGGCAAGAGCGAGCCTTGGACACTGGCTCTGGAAAATG
TCGTGGGCGCCAAGAACATGAATGTGCGGCCACTGCTGAACTACTTCGAGCCCCTGTTCACCTGGCTGAAGGACCA
GAACAAGAACAGCTTCGTCGGCTGGTCGACCGATTGGAGCCCTTATGCCGATCAGAGCATCAAAGTGCGGATCAGC
CTGAAGTCTGCCCTGGGCGATAGAGCCTACGAGTGGAACGACAACGAGATGTACCTGTTCCGGTCCAGCGTGGCAT
ATGCTATGCGGCAGTACTTCCTGAAAGTGAAGAACCAGATGATCCTGTTCGGCGAAGAGGACGTGCGCGTGGCCAA
TCTGAAGCCCAGAATCAGCTTCAACTTCTTCGTGACAGCCCCTAAGAACGTGTCCGATATCATCCCCAGAACCGAG
GTGGAAAAGGCCATCAGAATGAGCAGAAGCCGGATCAACGACGCCTTCCGGCTGAACGACAATAGCCTGGAATTTC
TGGGCATCCAGCCTACACTGGGCCCTCCAAATCAGCCTCCTGTTAGCGGAGGAGGAGGATCTGGAGGAGGAGGATC
CGCTAGCACAAAAGGCCCTAGCGTCTTTCCACTCGCCCCATCCTCCAAGAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAGCTT
GGGTACCCAGACCTATATCTGCAATGTCAATCATAAACCCAGTAACACAAAGGTAGACAAGAAAGTCGAACCCAAA
TCTTGCGACAAAACTCACACATGCCCTCCTTGCCCCGCCCCCGAGCTCCTGGGGGGACCTTCGGTTTTTCTTTTTC
CACCTAAACCGAAAGACACCCTGATGATCAGCAGGACACCAGAAGTGACATGTGTCGTCGTGGACGTATCCCATGA
GGACCCGGAGGTGAAGTTCAACTGGTATGTTGATGGTGTGGAAGTGCATAATGCAAAAACTAAGCCACGGGAGGAA
CAGTACAATAGCACATACAGAGTAGTTAGCGTTCTGACTGTCCTGCACCAAGATTGGCTGAACGGGAAGGAATACA
AGTGTAAGGTGAGCAACAAAGCCCTGCCCGCTCCAATTGAGAAAACAATTTCTAAGGCCAAAGGACAGCCCCGGGA
GCCACAGGTGTATACTCTGCCGCCTAGCAGGGAGGAAATGACCAAGAACCAGGTGAGCCTGACCTGTCTGGTGAAA
GGCTTCTACCCCAGCGATATCGCAGTTGAGTGGGAGAGCAATGGCCAACCCGAGAACAACTACAAGACTACGCCCC
CCGTGCTTGATAGCGACGGATCATTTTTCCTGTACTCAAAACTGACCGTGGACAAAAGCAGATGGCAGCAGGGAAA
CGTTTTCAGTTGCTCAGTGATGCACGAAGCGCTGCACAATCATTATACTCAGAAAAGCCTGAGCTTGAGCCCGGGA
AAA
SEQ ID NO: 21
EflACE2 light chain amino acid sequence
ETGSTIEEQAKYFLDKFNHEAEDLFYQSSLASWNYNTNITEENVQNMNNAGDKWSAFLKEQSTTAQMYPLQEIQNL
TVKLQLQALQQNGSSVLSEDKSKRINTILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDYSRGQLIEDVEHTFEEIKPLYEHLHAY
VRAKLMNAYPSYISPIGCLPAHLLGDMWGRFWTNLYSLTVPFGQKPNIDVTDAMVDQAWDAQRIFKEAEKFFVSVG
LPNMTQGFWEYSMLTDPGNVQKAVCHPTAWDLGKGDFRILMCTKVTMDDFLTAHHEMGHIQYDMAYAAQPFLLRNG
ANEGFHEAVGEIMSLSAATPKHLKSIGLLSPDFQEDNETEINFLLKQALTIVGTLPFTYMLEKWRWMVFKGEIPKD
QWMKKWWEMKREIVGVVEPVPHDETYCDPASLFHVSNDYSFIRYYTRTLYQFQFQEALCQAAKHEGPLHKCDISNS
TEAGQKLENMLRLGKSEPWTLALENVVGAKNMNVRPLLNYFEPLFTWLKDQNKNSFVGWSTDWSPYADQSIKVRIS
LKSALGDRAYEWNDNEMYLFRSSVAYAMRQYFLKVKNQMILFGEEDVRVANLKPRISENFFVTAPKNVSDIIPRTE
VEKAIRMSRSRINDAFRLNDNSLEFLGIQPTLGPPNQPPVSGGGGSGGGGSRTVAAPSVFIFPPSDEQLKSGTASV
VCLLNNFYPREAKVQWKVDNALQSGNSQESVTEQDSKDSTYSLSSTLTLSKADYEKHKVYACEVTHQGLSSPVTKS
FNRGEC
SEQ ID NO: 22
EflACE2 light chain nucleotide sequence
GAAACCGGTTCCACCATTGAGGAACAGGCCAAGTACTTTTTGGACAAGTTTAACCACGAAGCCGAAGACCTGTTCT
ATCAAAGTTCACTTGCTTCTTGGAATTATAACACCAATATTACTGAAGAGAATGTCCAAAACATGAACAACGCCGG
CGACAAGTGGAGCGCCTTCCTGAAAGAGCAGAGCACAACAGCCCAGATGTACCCTCTGCAAGAGATCCAGAACCTG
ACCGTGAAGCTCCAGCTGCAGGCCCTCCAGCAGAATGGAAGCTCTGTGCTGAGCGAGGACAAGAGCAAGCGGCTGA
ACACCATCCTGAATACCATGAGCACCATCTACAGCACCGGCAAAGTGTGCAACCCCGACAATCCCCAAGAGTGCCT
GCTGCTGGAACCCGGCCTGAATGAGATCATGGCCAACAGCCTGGACTACAACGAGAGACTGTGGGCCTGGGAGTCT
TGGAGAAGCGAAGTGGGAAAGCAGCTGCGGCCCCTGTACGAGGAATACGTGGTGCTGAAGAACGAGATGGCCAGAG
CCAACCACTACGAGGACTACGGCGACTATTGGAGAGGCGACTACGAAGTGAATGGCGTGGACGGCTACGACTACAG
CAGAGGCCAGCTGATCGAGGACGTGGAACACACCTTCGAGGAAATCAAGCCTCTGTACGAGCATCTGCACGCCTAC
GTGCGGGCCAAGCTGATGAATGCTTACCCCAGCTACATCAGCCCCATCGGCTGTCTGCCTGCTCATCTGCTGGGAG
ACATGTGGGGCAGATTCTGGACCAACCTGTACAGCCTGACAGTGCCCTTCGGCCAGAAACCTAACATCGACGTGAC
CGACGCTATGGTGGATCAGGCTTGGGATGCCCAGCGGATCTTCAAAGAGGCCGAGAAGTTCTTCGTGTCCGTGGGC
CTGCCTAATATGACCCAAGGCTTCTGGGAGTACTCCATGCTGACAGACCCCGGCAACGTGCAGAAAGCCGTGTGTC
ATCCTACCGCCTGGGATCTCGGCAAGGGCGACTTCAGAATCCTGATGTGCACCAAAGTGACGATGGACGACTTCCT
GACAGCCCACCACGAGATGGGCCACATCCAGTACGATATGGCCTACGCCGCTCAGCCCTTCCTGCTGAGAAATGGC
GCCAATGAGGGCTTCCATGAGGCCGTGGGAGAGATCATGAGCCTGTCTGCCGCCACACCTAAGCACCTGAAGTCTA
TCGGACTGCTGAGCCCCGACTTCCAAGAGGACAACGAGACAGAGATCAACTTCCTGCTCAAGCAGGCCCTGACCAT
CGTGGGCACACTGCCCTTTACCTACATGCTGGAAAAGTGGCGGTGGATGGTCTTTAAGGGCGAGATCCCCAAGGAC
CAGTGGATGAAGAAATGGTGGGAGATGAAGCGCGAGATCGTGGGCGTTGTGGAACCTGTGCCTCACGACGAGACAT
ACTGCGATCCTGCCAGCCTGTTTCACGTGTCCAACGACTACTCCTTCATCCGGTACTACACCCGGACACTGTACCA
GTTCCAGTTTCAAGAGGCTCTGTGCCAGGCCGCCAAGCACGAAGGACCTCTGCACAAGTGCGACATCAGCAACTCT
ACAGAGGCCGGACAGAAACTGTTCAACATGCTGCGGCTGGGCAAGAGCGAGCCTTGGACACTGGCTCTGGAAAATG
TCGTGGGCGCCAAGAACATGAATGTGCGGCCACTGCTGAACTACTTCGAGCCCCTGTTCACCTGGCTGAAGGACCA
GAACAAGAACAGCTTCGTCGGCTGGTCGACCGATTGGAGCCCTTATGCCGATCAGAGCATCAAAGTGCGGATCAGC
CTGAAGTCTGCCCTGGGCGATAGAGCCTACGAGTGGAACGACAACGAGATGTACCTGTTCCGGTCCAGCGTGGCAT
ATGCTATGCGGCAGTACTTCCTGAAAGTGAAGAACCAGATGATCCTGTTCGGCGAAGAGGACGTGCGCGTGGCCAA
TCTGAAGCCCAGAATCAGCTTCAACTTCTTCGTGACAGCCCCTAAGAACGTGTCCGATATCATCCCCAGAACCGAG
GTGGAAAAGGCCATCAGAATGAGCAGAAGCCGGATCAACGACGCCTTCCGGCTGAACGACAATAGCCTGGAATTTC
TGGGCATCCAGCCTACACTGGGCCCTCCAAATCAGCCTCCTGTTAGCGGAGGAGGAGGATCTGGAGGAGGAGGCTC
GAGGACAGTGGCCGCTCCTAGCGTGTTCATCTTTCCACCTAGCGACGAGCAGCTGAAGTCTGGCACAGCCTCTGTC
GTGTGCCTGCTGAACAACTTCTACCCCAGAGAAGCCAAGGTGCAGTGGAAGGTGGACAACGCCCTGCAGAGCGGCA
ATAGCCAAGAGAGCGTGACCGAGCAGGACAGCAAGGACTCTACCTACAGCCTGAGCAGCACCCTGACACTGAGCAA
GGCCGACTACGAGAAGCACAAAGTGTACGCCTGCGAAGTGACCCACCAGGGCCTTTCTAGCCCTGTGACCAAGAGC
TTCAACCGGGGCGAGTGC
SEQ ID NO: 23
EflACE2-Ab-like-H429F H chain amino acid sequence
ETGSTIEEQAKYFLDKFNHEAEDLFYQSSLASWNYNTNITEENVQNMNNAGDKWSAFLKEQSTTAQMYPLQEIQNL
TVKLQLQALQQNGSSVLSEDKSKRLNTILNTMSTIYSTGKVCNPDNPQECLLLEPGLNEIMANSLDYNERLWAWES
WRSEVGKQLRPLYEEYVVLKNEMARANHYEDYGDYWRGDYEVNGVDGYDYSRGQLIEDVEHTFEEIKPLYEHLHAY
VRAKLMNAYPSYISPIGCLPAHLLGDMWGRFWTNLYSLTVPFGQKPNIDVTDAMVDQAWDAQRIFKEAEKFFVSVG
LPNMTQGFWEYSMLTDPGNVQKAVCHPTAWDLGKGDFRILMCTKVTMDDFLTAHHEMGHIQYDMAYAAQPFLLRNG
ANEGFHEAVGEIMSLSAATPKHLKSIGLLSPDFQEDNETEINFLLKQALTIVGTLPFTYMLEKWRWMVFKGEIPKD
QWMKKWWEMKREIVGVVEPVPHDETYCDPASLFHVSNDYSFIRYYTRTLYQFQFQEALCQAAKHEGPLHKCDISNS
TEAGQKLENMLRLGKSEPWTLALENVVGAKNMNVRPLLNYFEPLFTWLKDQNKNSFVGWSTDWSPYADQSIKVRIS
LKSALGDRAYEWNDNEMYLERSSVAYAMRQYFLKVKNQMILFGEEDVRVANLKPRISENFFVTAPKNVSDIIPRTE
VEKAIRMSRSRINDAFRLNDNSLEFLGIQPTLGPPNQPPVSGGGGSGGGGSASTKGPSVFPLAPSSKSTSGGTAAL
GCLVKDYFPEPVTVSWNSGALTSGVHTFPAVLQSSGLYSLSSVVTVPSSSLGTQTYICNVNHKPSNTKVDKKVEPK
SCDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSREEMTKNQVSLTCLVK
GFYPSDIAVEWESNGQPENNYKTTPPVLDSDGSFFLYSKLTVDKSRWQQGNVFSCSVMFEALHNHYTQKSLSLSPG
K
SEQ ID NO: 24 EflACE2-Ab-like-H429F H chain nucleotide sequence
GAAACCGGTTCCACCATTGAGGAACAGGCCAAGTACTTTTTGGACAAGTTTAACCACGAAGCCGAAGACCTGTTCT
ATCAAAGTTCACTTGCTTCTTGGAATTATAACACCAATATTACTGAAGAGAATGTCCAAAACATGaACAACGCCGG
CGACAAGTGGAGCGCCTTCCTGAAAGAGCAGAGCACAACAGCCCAGATGTACCCTCTGCAAGAGATCCAGAACCTG
ACCGTGAAGCTCCAGCTGCAGGCCCTCCAGCAGAATGGAAGCTCTGTGCTGAGCGAGGACAAGAGCAAGCGGCTGA
ACACCATCCTGAATACCATGAGCACCATCTACAGCACCGGCAAAGTGTGCAACCCCGACAATCCCCAAGAGTGCCT
GCTGCTGGAACCCGGCCTGAATGAGATCATGGCCAACAGCCTGGACTACAACGAGAGACTGTGGGCCTGGGAGTCT
TGGAGAAGCGAAGTGGGAAAGCAGCTGCGGCCCCTGTACGAGGAATACGTGGTGCTGAAGAACGAGATGGCCAGAG
CCAACCACTACGAGGACTACGGCGACTATTGGAGAGGCGACTACGAAGTGAATGGCGTGGACGGCTACGACTACAG
CAGAGGCCAGCTGATCGAGGACGTGGAACACACCTTCGAGGAAATCAAGCCTCTGTACGAGCATCTGCACGCCTAC
GTGCGGGCCAAGCTGATGAATGCTTACCCCAGCTACATCAGCCCCATCGGCTGTCTGCCTGCTCATCTGCTGGGAG
ACATGTGGGGCAGATTCTGGACCAACCTGTACAGCCTGACAGTGCCCTTCGGCCAGAAACCTAACATCGACGTGAC
CGACGCTATGGTGGATCAGGCTTGGGATGCCCAGCGGATCTTCAAAGAGGCCGAGAAGTTCTTCGTGTCCGTGGGC
CTGCCTAATATGACCCAAGGCTTCTGGGAGTACTCCATGCTGACAGACCCCGGCAACGTGCAGAAAGCCGTGTGTC
ATCCTACCGCCTGGGATCTCGGCAAGGGCGACTTCAGAATCCTGATGTGCACCAAAGTGACGATGGACGACTTCCT
GACAGCCCACCACGAGATGGGCCACATCCAGTACGATATGGCCTACGCCGCTCAGCCCTTCCTGCTGAGAAATGGC
GCCAATGAGGGCTTCCATGAGGCCGTGGGAGAGATCATGAGCCTGTCTGCCGCCACACCTAAGCACCTGAAGTCTA
TCGGACTGCTGAGCCCCGACTTCCAAGAGGACAACGAGACAGAGATCAACTTCCTGCTCAAGCAGGCCCTGACCAT
CGTGGGCACACTGCCCTTTACCTACATGCTGGAAAAGTGGCGGTGGATGGTCTTTAAGGGCGAGATCCCCAAGGAC
CAGTGGATGAAGAAATGGTGGGAGATGAAGCGCGAGATCGTGGGCGTTGTGGAACCTGTGCCTCACGACGAGACAT
ACTGCGATCCTGCCAGCCTGTTTCACGTGTCCAACGACTACTCCTTCATCCGGTACTACACCCGGACACTGTACCA
GTTCCAGTTTCAAGAGGCTCTGTGCCAGGCCGCCAAGCACGAAGGACCTCTGCACAAGTGCGACATCAGCAACTCT
ACAGAGGCCGGACAGAAACTGTTCAACATGCTGCGGCTGGGCAAGAGCGAGCCTTGGACACTGGCTCTGGAAAATG
TCGTGGGCGCCAAGAACATGAATGTGCGGCCACTGCTGAACTACTTCGAGCCCCTGTTCACCTGGCTGAAGGACCA
GAACAAGAACAGCTTCGTCGGCTGGTCGACCGATTGGAGCCCTTATGCCGATCAGAGCATCAAAGTGCGGATCAGC
CTGAAGTCTGCCCTGGGCGATAGAGCCTACGAGTGGAACGACAACGAGATGTACCTGTTCCGGTCCAGCGTGGCAT
ATGCTATGCGGCAGTACTTCCTGAAAGTGAAGAACCAGATGATCCTGTTCGGCGAAGAGGACGTGCGCGTGGCCAA
TCTGAAGCCCAGAATCAGCTTCAACTTCTTCGTGACAGCCCCTAAGAACGTGTCCGATATCATCCCCAGAACCGAG
GTGGAAAAGGCCATCAGAATGAGCAGAAGCCGGATCAACGACGCCTTCCGGCTGAACGACAATAGCCTGGAATTTC
TGGGCATCCAGCCTACACTGGGCCCTCCAAATCAGCCTCCTGTTAGCGGAGGAGGAGGATCTGGAGGAGGAGGATC
CGCTAGCACAAAAGGCCCTAGCGTCTTTCCACTCGCCCCATCCTCCAAGAGCACCTCTGGGGGCACTGCGGCTTTG
GGCTGCTTGGTGAAGGATTACTTCCCAGAGCCTGTCACTGTCAGCTGGAATAGCGGGGCTCTGACCAGTGGAGTGC
ACACCTTTCCCGCCGTGTTACAGAGCAGCGGCCTCTATAGCCTGAGCAGTGTGGTAACTGTGCCCTCGAGCAGCTT
GGGTACCCAGACCTATATCTGCAATGTCAATCATAAACCCAGTAACACAAAGGTAGACAAGAAAGTCGAACCCAAA
TCTTGCGACAAAACTCACACATGCCCTCCTTGCCCCGCCCCCGAGCTCCTGGGGGGACCTTCGGTTTTTCTTTTTC
CACCTAAACCGAAAGACACCCTGATGATCAGCAGGACACCAGAAGTGACATGTGTCGTCGTGGACGTATCCCATGA
GGACCCGGAGGTGAAGTTCAACTGGTATGTTGATGGTGTGGAAGTGCATAATGCAAAAACTAAGCCACGGGAGGAA
CAGTACAATAGCACATACAGAGTAGTTAGCGTTCTGACTGTCCTGCACCAAGATTGGCTGAACGGGAAGGAATACA
AGTGTAAGGTGAGCAACAAAGCCCTGCCCGCTCCAATTGAGAAAACAATTTCTAAGGCCAAAGGACAGCCCCGGGA
GCCACAGGTGTATACTCTGCCGCCTAGCAGGGAGGAAATGACCAAGAACCAGGTGAGCCTGACCTGTCTGGTGAAA
GGCTTCTACCCCAGCGATATCGCAGTTGAGTGGGAGAGCAATGGCCAACCCGAGAACAACTACAAGACTACGCCCC
CCGTGCTTGATAGCGACGGATCATTTTTCCTGTACTCAAAACTGACCGTGGACAAAAGCAGATGGCAGCAGGGAAA
CGTTTTCAGTTGCTCAGTGATGTTCGAAGCGCTGCACAATCATTATACTCAGAAAAGCCTGAGCTTGAGCCCGGGA
AAA

1. An immunotherapeutic protein comprising an angiotensin converting enzyme 2 (ACE2) polypeptide or a fragment thereof linked to a polypeptide comprising an Fe region component, wherein said immunotherapeutic protein is capable of binding to a coronavirus spike protein (S protein).

2. The immunotherapeutic protein of claim 1 comprising a fragment of ACE2 comprising all or a portion of the ACE2 ectodomain.

3. The immunotherapeutic protein of claim 2, wherein the portion of the ACE2 ectodomain includes a collectrin domain.

4. The immunotherapeutic protein of claim 2, wherein the portion of the ACE2 ectodomain excludes a collectrin domain.

5. The immunotherapeutic protein of claim 2 or 3, wherein the portion of the ACE2 ectodomain comprises the amino acid sequence of amino acids 19 to 740 of SEQ ID NO:1 or an amino acid sequence showing 98% sequence identity to the amino acid sequence of amino acids 19 to 740 of SEQ ID NO:1.

6. The immunotherapeutic protein of claim 2 or 4, wherein the portion of the ACE2 ectodomain comprises the amino acid sequence of amino acids 19 to 615 of SEQ ID NO:1 or an amino acid sequence showing ≥98% sequence identity to the amino acid sequence of amino acids 19 to 615 of SEQ ID NO:1.

7. The immunotherapeutic protein of any one of claims 1 to 6, wherein ACE2 polypeptide or a fragment thereof comprises a triple mutation of T27Y, L79T and N330Y.

8. An immunotherapeutic protein comprising a cell surface receptor polypeptide or co-receptor polypeptide or fragment thereof, wherein said cell surface receptor polypeptide is a cellular entry receptor for the entry of a virus into a host cell, linked to a polypeptide comprising an Fc region component, wherein said immunotherapeutic protein is capable of binding to a structural protein of said virus.

9. The immunotherapeutic protein of claim 8 comprising a fragment of the cell surface receptor polypeptide comprising all or a portion of an ectodomain of the cell surface receptor.

10. The immunotherapeutic protein of claim 8 or 9, wherein the cell surface receptor polypeptide is selected from Hsp70, hepatitis A virus cellular receptor 1 (HAVCR1/TIM-1), cluster of differentiation 155 (CD155), intracellular adhesion molecule 1 (ICAM-1), Insulin-like growth factor-1 receptor (IGF1R), glucose transporter 1 (GLUT1), cluster of differentiation 4 receptor (CD4 receptor) and dipeptidyl peptidase 4 (DPP4), and fragments thereof.

11. The immunotherapeutic protein of claim 8 comprising a co-receptor polypeptide selected from C—X—C chemokine receptor type 4 (CXCR4), C—C chemokine receptor type 5 (CCR5), nucleolin and occludin, or fragments thereof.

12. The immunotherapeutic protein of any one of claims 1 to 11 wherein the Fe region component comprises at least a CH3 domain (or at least a CH4 domain).

13. The immunotherapeutic protein of any one of claims 1 to 11, wherein the Fc region component comprises a full-length Fc region polypeptide comprising the constant heavy domain 2 (CH2), constant heavy domain 3 (CH3) and hinge sequences.

14. The immunotherapeutic protein of any one of claims 1 to 13, wherein the Fc region component comprises modified glycosylation.

15. The immunotherapeutic protein of any one of claims 1 to 14, wherein the Fc region component is derived from an IgG1 heavy chain polypeptide.

16. The immunotherapeutic protein of any one of claims 1 to 15, wherein the immunotherapeutic protein is provided in the form of a fusion protein.

17. The immunotherapeutic protein of any one of claims 1 to 16, wherein the immunotherapeutic protein is oligomeric.

18. The immunotherapeutic protein of claim 17, wherein the Fc region component comprises an amino acid substitution at the position corresponding to H429 of the amino acid sequence of the human IgG1 heavy chain polypeptide (EU numbering).

19. The immunotherapeutic protein of claim 18, wherein the amino acid substitution is H429X¹, where X¹ is selected from tyrosine (H429Y), methionine (11429M), isoleucine (H429I), leucine (H429L), tryptophan (H429W) and valine (H429V).

20. The immunotherapeutic protein of claim 19, wherein the Fc region component comprises an H429Y amino acid substitution.

21. The immunotherapeutic protein of claim 1, wherein the immunotherapeutic protein comprises an ACE2 polypeptide (or a fragment thereof) linked to a polypeptide comprising an Fc region component, wherein said Fc region component comprises an amino acid substitution at the position corresponding to H429 of the amino acid sequence of the human IgG1 heavy chain polypeptide (EU numbering).

22. The immunotherapeutic protein of claim 21, wherein the amino acid substitution is H429X¹, where X¹ is selected from tyrosine (H429Y), methionine (H429M), isoleucine (H429I), leucine (H429L), tryptophan (H429W) and valine (H429V).

23. The immunotherapeutic protein of claim 22, wherein the Fc region component comprises an H429Y amino acid substitution.

24. The immunotherapeutic protein of claim 21, wherein the amino acid substitution is H429X², where X² is selected from phenylalanine (H429F), glutamate (H429E), glutamine (H429Q) and serine (H429S).

25. The immunotherapeutic protein of claim 24, wherein the Fc region component comprises an H429F amino acid substitution.

26. The immunotherapeutic protein of any one of claims 1 to 7, wherein the immunotherapeutic protein is an antibody-like molecule in an H₂ or H₂L₂ format.

27. The immunotherapeutic protein of claim 25, wherein the immunotherapeutic protein has an H₂L₂ format and comprises two fusion proteins each comprising an immunoglobulin heavy chain polypeptide which comprises an Fc region component comprising at least a constant heavy chain domain 3 (CH3) domain (or at least a constant heavy domain 4 (CH4) domain) and including an amino acid substitution at a position corresponding to H429 of the amino acid sequence of human IgG1 heavy chain (Eu numbering) and said ACE2 polypeptide linked thereto, and two further fusion proteins each comprising an immunoglobulin light chain polypeptide and said ACE2 polypeptide linked thereto.

28. The immunotherapeutic protein of any one of claims 8 to 11, wherein the immunotherapeutic protein is an antibody-like molecule in an H₂ or H₂L₂ format.

29. The immunotherapeutic protein of claim 28, wherein the immunotherapeutic protein has an H₂L₂ format and comprises two fusion proteins each comprising an immunoglobulin heavy chain polypeptide which comprises an Fc region component comprising at least a constant heavy chain domain 3 (CH3) domain (or at least a constant heavy domain 4 (CH4) domain) and including an amino acid substitution at a position corresponding to H429 of the amino acid sequence of human IgG1 heavy chain (Eu numbering) and a cell surface receptor polypeptide or co-receptor polypeptide or fragment thereof linked thereto, and two further fusion proteins each comprising an immunoglobulin light chain polypeptide and a cell surface receptor polypeptide or co-receptor polypeptide or fragment thereof linked thereto.

30. The immunotherapeutic protein of claim 29, wherein the cell surface receptor polypeptide or co-receptor polypeptide or fragment thereof of each of the total of four fusion proteins may be the same or different.

31. An expression construct comprising a polynucleotide sequence encoding the immunotherapeutic protein of any one of claims 1 to 30, or a host cell comprising said expression construct for the expression of the immunotherapeutic protein of any one of claims 1 to 30.

32. A method for the treatment and/or the prevention of a viral infection in a subject, comprising administering to the subject an effective amount of the immunotherapeutic protein of any one of claims 1 to 30.

33. The method of claim 32, further comprising administering an antibody directed against a virus causing the viral infection.

34. The method of claim 33, wherein the antibody comprises an Fe region component comprising an amino acid substitution at the position corresponding to H429 of the amino acid sequence of the human IgG1 heavy chain polypeptide (EU numbering).

35. The method of claim 34, wherein the amino acid substitution is H429X², where X² is selected from phenylalanine (H429F), glutamate (H429E), glutamine (H429Q) and serine (H429S).

36. The method of any one of claims 32 to 34, wherein the antibody is an anti-SARS-CoV-2 antibody, and the immunotherapeutic protein comprises a fusion protein comprising an ACE2 polypeptide or a fragment thereof linked to a polypeptide comprising an Fe region component which comprises an amino acid substitution at the position corresponding to H429 of the amino acid sequence of the human IgG1 heavy chain polypeptide (EU numbering).

37. The method of claim 36, wherein the Fc region component of the immunotherapeutic protein comprises an H429F amino acid substitution.

38. Use of the immunotherapeutic protein of any one of claims 1 to 30, for the treatment and/or the prevention of a viral infection in a subject.

39. Use of an immunotherapeutic protein of any one of claims 1 to 30, in the manufacture of a medicament for the treatment and/or the prevention of a viral infection in a subject.

40. A pharmaceutical composition or medicament comprising an immunotherapeutic protein of any one of claims 1 to 30, and a pharmaceutically acceptable carrier, diluent and/or excipient.

41. A method of neutralising virus in a subject with a viral infection, comprising administering to the subject an effective amount of an immunotherapeutic protein comprising an angiotensin converting enzyme 2 (ACE2) polypeptide or a fragment thereof linked to a polypeptide comprising an Fc region component, wherein said immunotherapeutic protein is capable of binding to a coronavirus spike protein (S protein) to inhibit viral infection of a host cell via an ACE2 cell surface receptor of said host cell, and wherein said immunotherapeutic protein is a soluble oligomer at physiological pH.

42. The method of claim 41, wherein said immunotherapeutic protein is hexameric at physiological pH.

43. The method of claim 41 or 42, wherein the Fc region component comprises one or more mutation enabling self-association of monomers into an oligomeric form at physiological pH.

44. The method of claim 43, wherein the one or more mutation is an amino acid substitution at the position corresponding to H429 of the amino acid sequence of the human IgG1 heavy chain polypeptide (EU numbering).

45. The method of claim 44, wherein the amino acid substitution is H429X¹, where X¹ is selected from tyrosine (H429Y), methionine (H429M), isoleucine (H429I), leucine (H429L), tryptophan (H429W) and valine (H429V).

46. The method of claim 45, wherein the Fc region component comprises an H429Y amino acid substitution.

47. The method of any one of claims 41 to 46, wherein the ACE2 polypeptide or a fragment thereof comprises a triple amino acid substitution of T27Y, L79T and N330Y.

48. The method of any one of claims 41 to 46, wherein the ACE2 polypeptide or a fragment thereof comprises an amino acid substitution selected from the group consisting of D30E, K31F, N33D, H341, H34S and E35Q.

49. The method of any one of claims 41 to 48, wherein the immunotherapeutic protein shows abrogated FcγR binding and activation.

50. A method of eliciting complement-dependent cytotoxicity to treat a viral infection in a subject, comprising administering to the subject an effective amount of an immunotherapeutic protein comprising an angiotensin converting enzyme 2 (ACE2) polypeptide or a fragment thereof linked to a polypeptide comprising an Fc region component, wherein said immunotherapeutic protein is capable of binding to a coronavirus spike protein (S protein) to inhibit viral infection of a host cell via an ACE2 cell surface receptor of said host cell, and wherein said immunotherapeutic protein form oligomers upon binding to a spike protein of a coronavirus to achieve enhanced capability for complement activation and thereby complement-dependent cytotoxicity (CDC) of cells infected with coronavirus or undergoing infection with coronavirus.

51. The method of claim 50, further comprising administering an antibody directed against coronavirus.

52. The method of claim 50 or 51, wherein the immunotherapeutic protein comprises a polypeptide comprising an Fc region component comprising an amino acid mutation at a position(s) corresponding to H429 and, optionally, K447 of the amino acid sequence of the human IgG1 heavy chain polypeptide (EU numbering).

53. The method of claim 52, wherein the amino acid mutation is H429X², where X² is selected from phenylalanine (H429F), glutamate (H429E), glutamine (H429Q) and serine (H429S), and optionally, K447X³, where X³ is selected from null (K447del), and glutamate (K447E).

54. The method of claim 53, wherein the amino acid mutation is H429F and, optionally, K447del (EU numbering).

55. The method of any one of claims 50 to 54, wherein the ACE2 polypeptide or a fragment thereof comprises a portion of the ACE2 ectodomain which excludes a collectrin domain.

56. A method of producing an immunotherapeutic protein of any one of claims 1 to 30, comprising culturing a host cell comprising a construct encoding said protein under conditions suitable for the expression of said protein, and recovering the protein from culture supernatant under conditions of:
(i) mildly acidic pH to recover immunotherapeutic protein in a monomeric form; or
(ii) substantially neutral pH to recover immunotherapeutic protein in an oligomeric form.

57. A method of producing an immunotherapeutic protein of any one of claims 1 to 30, comprising culturing a host cell comprising a construct encoding said protein under conditions suitable for the expression of said protein, and recovering the protein from culture supernatant using a method comprising affinity chromatography using an elution buffer comprising a concentration of arginine of less than 130 mM and at less than or equal to pH 5.0.

58. The method of claim 56, wherein the recovery of expressed immunotherapeutic protein comprises recovery by size exclusion chromatography (SEC).


SEQUENCE LISTING (24 sequences)

SEQ ID NO: 1 - protein, 955 aa, synthetic construct
ETGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 832 aa, synthetic construct
ETGSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGSGSGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 5 aa, Homo sapiens
KGDFR

SEQ ID NO: 4 - protein, 5 aa, synthetic construct
GGGGS

SEQ ID NO: 5 - protein, 31 aa, Homo sapiens
MGILPSPGMPALLSLVSLLSVLLMGCVAETG

SEQ ID NO: 6 - protein, 7 aa, synthetic construct
GSGSGSG

SEQ ID NO: 7 - protein, 448 aa, synthetic construct
EVQLVQSGAEVKKPGASVKVSCKASGYTFTSQYMHWVRQAPGQGLEWIGIINPSGVHTSYAQKFQGRVTLTRDTSTSTLYMELSSLRSEDTAVYYCARGSPKGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 1344 bp, synthetic construct
  source 1..1344: mol_type = other DNA
gaagttcagctggttcagtctggcgccgaagtgaagaaacctggcgcctctgtgaaggtgtcctgcaaggccagcggctacacctttaccagccagtacatgcactgggtccgacaggctccaggacagggactcgagtggatcggcatcatcaatcctagcggcgtgcacaccagctacgcccagaaattccagggcagagtgaccctgaccagagacaccagcaccagcacactgtacatggaactgagcagcctgcggagcgaggataccgccgtgtactattgtgccagaggctctcctaagggcgccttcgattattggggccagggcacactggtcaccgtgtcctctgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 9 - protein, 217 aa, synthetic construct
EIVMMQSPGTLSLSPGERATLSCRASQSVRSNYLAWYQQKPGQAPRLLIYGASSRATGIPDRFSGSGSGTDFTLTISRLEPEDFAVYYCQQYGSSPPRFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - DNA, 651 bp, synthetic construct
  source 1..651: mol_type = other DNA
gagatcgtcatgatgcagagccccggcacactgtcactgtctccaggcgaaagagccacactgagctgtagagcctctcagagcgtgcggagcaattacctggcctggtatcagcagaagcccggacaggctcctcggctgttgatctatggcgcctctagcagagccactggcatccccgatagattttctggcagcggctccggcaccgacttcaccctgacaatcagcagactggaacccgaggacttcgccgtgtactactgtcagcagtacggcagcagccctcctcggttcacatttggccctggtaccaaggtggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 11 - protein, 447 aa, synthetic construct
QVQEVQLLESGGGLVQPGGSLRLSCAASGFTFSSYVMTWARQAPGKGLEWVSAISGTGYTYYADSVKGRFTVSRDNSKNTLFLQMSSLRAEDTAVYYCAITMAPVVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 455 aa, synthetic construct
QVQLVESGAEVKKPGESLKISCKGSGYTFTRYWIGWVRQMPGKGLEWMGIIYPGDSDTRYSPSFQGHVTISADKSISTAYLQWNSLKASDTAMYYCARLPQYCSNGVCQRWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1341 bp, synthetic construct
  source 1..1341: mol_type = other DNA
caagtgcaggaagtgcagctgctggaatctggcggaggactggttcaaccaggcggctctctgagactgtcttgtgccgccagcggcttcaccttcagcagctacgttatgacctgggccagacaggcccctggcaaaggacttgaatgggtgtccgccatcagcggcacaggctacacctactacgccgactctgtgaagggcagattcaccgtgtccagagacaacagcaagaataccctgttcctgcagatgagcagcctgagagccgaggataccgccgtgtactactgcgccattacaatggcccctgtcgtgtggggccagggcacaacagtgacagtcagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 14 - DNA, 1365 bp, synthetic construct
  source 1..1365: mol_type = other DNA
caagtgcagctggttgaatctggcgccgaagtgaagaagcctggcgagagcctgaagatcagctgcaaaggcagcggctacaccttcaccagatactggatcggctgggtccgacagatgcctggcaaaggccttgagtggatgggcatcatctaccccggcgacagcgataccagatacagccctagctttcagggccacgtgaccatcagcgccgacaagtctatcagcaccgcctacctgcagtggaacagcctgaaggccagcgacaccgccatgtactactgtgccagactgccccagtactgcagcaatggcgtttgccagcgttggttcgatccttggggacagggcacactggtcaccgttagcagcgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgaactcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 15 - protein, 216 aa, synthetic construct
SYELTQPPSVSVSPGQTARITCSGDALPKRYAYWYQQKSGQAPILVIYEDKKRPSGIPERLSGSKSGTVATLTISGAQVEDEADYYCYSTDSSGNHAVFGGGTQLTVLGQPKAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 214 aa, synthetic construct
EIVLTQSPSSVSASVGDRVTITCRASQGISSWLAWYQQKPGKAPKLLIYAASSLQSGVPSRFSGSGSGTDFTLTISSLQPEDFATYYCQQGNSFPYTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - DNA, 648 bp, synthetic construct
  source 1..648: mol_type = other DNA
agctatgaactgacacagcctccaagcgtgtccgtgtctcctggacagaccgccagaatcacatgtagcggagatgccctgcctaagagatacgcctactggtatcagcagaagtccggacaggcccctatcctggtcatctacgaggacaagaagcggccctctggcatccctgagagactgagcggaagcaagtctggcacagtggccacactgacaatctctggcgcccaggtggaagatgaggccgactactactgctacagcaccgacagctctggcaaccacgctgtgtttggcggaggtacccaactgaccgtgctgggccaacctaaagccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 18 - DNA, 642 bp, synthetic construct
  source 1..642: mol_type = other DNA
gaaattgtgctgacacagagccccagcagcgtgtcagcctctgtgggagacagagtgaccatcacctgtagagccagccagggcatctcttcttggctggcctggtatcagcagaagcctggcaaggcccctaagctgctgatctatgccgctagctctctgcagtctggcgtgcccagcagattttctggcagcggctctggcaccgacttcaccctgaccatatctagcctgcagcctgaggacttcgccacctactattgccagcagggcaacagcttcccctacacctttggccagggtaccaacctggaaatcaagcggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 19 - protein, 1065 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 3195 bp, synthetic construct
  source 1..3195: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggatccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgagctcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgcacgaagcgctgcacaatcattatactcagaaaagcctgagcttgagcccgggaaaa

SEQ ID NO: 21 - protein, 842 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - DNA, 2526 bp, synthetic construct
  source 1..2526: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggctcgaggacagtggccgctcctagcgtgttcatctttccacctagcgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgagtgc

SEQ ID NO: 23 - protein, 1065 aa, synthetic construct
ETGSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDRAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSGGGGSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMFEALHNHYTQKSLSLSPGK

SEQ ID NO: 24 - DNA, 3195 bp, synthetic construct
  source 1..3195: mol_type = other DNA
gaaaccggttccaccattgaggaacaggccaagtactttttggacaagtttaaccacgaagccgaagacctgttctatcaaagttcacttgcttcttggaattataacaccaatattactgaagagaatgtccaaaacatgaacaacgccggcgacaagtggagcgccttcctgaaagagcagagcacaacagcccagatgtaccctctgcaagagatccagaacctgaccgtgaagctccagctgcaggccctccagcagaatggaagctctgtgctgagcgaggacaagagcaagcggctgaacaccatcctgaataccatgagcaccatctacagcaccggcaaagtgtgcaaccccgacaatccccaagagtgcctgctgctggaacccggcctgaatgagatcatggccaacagcctggactacaacgagagactgtgggcctgggagtcttggagaagcgaagtgggaaagcagctgcggcccctgtacgaggaatacgtggtgctgaagaacgagatggccagagccaaccactacgaggactacggcgactattggagaggcgactacgaagtgaatggcgtggacggctacgactacagcagaggccagctgatcgaggacgtggaacacaccttcgaggaaatcaagcctctgtacgagcatctgcacgcctacgtgcgggccaagctgatgaatgcttaccccagctacatcagccccatcggctgtctgcctgctcatctgctgggagacatgtggggcagattctggaccaacctgtacagcctgacagtgcccttcggccagaaacctaacatcgacgtgaccgacgctatggtggatcaggcttgggatgcccagcggatcttcaaagaggccgagaagttcttcgtgtccgtgggcctgcctaatatgacccaaggcttctgggagtactccatgctgacagaccccggcaacgtgcagaaagccgtgtgtcatcctaccgcctgggatctcggcaagggcgacttcagaatcctgatgtgcaccaaagtgacgatggacgacttcctgacagcccaccacgagatgggccacatccagtacgatatggcctacgccgctcagcccttcctgctgagaaatggcgccaatgagggcttccatgaggccgtgggagagatcatgagcctgtctgccgccacacctaagcacctgaagtctatcggactgctgagccccgacttccaagaggacaacgagacagagatcaacttcctgctcaagcaggccctgaccatcgtgggcacactgccctttacctacatgctggaaaagtggcggtggatggtctttaagggcgagatccccaaggaccagtggatgaagaaatggtgggagatgaagcgcgagatcgtgggcgttgtggaacctgtgcctcacgacgagacatactgcgatcctgccagcctgtttcacgtgtccaacgactactccttcatccggtactacacccggacactgtaccagttccagtttcaagaggctctgtgccaggccgccaagcacgaaggacctctgcacaagtgcgacatcagcaactctacagaggccggacagaaactgttcaacatgctgcggctgggcaagagcgagccttggacactggctctggaaaatgtcgtgggcgccaagaacatgaatgtgcggccactgctgaactacttcgagcccctgttcacctggctgaaggaccagaacaagaacagcttcgtcggctggtcgaccgattggagcccttatgccgatcagagcatcaaagtgcggatcagcctgaagtctgccctgggcgatagagcctacgagtggaacgacaacgagatgtacctgttccggtccagcgtggcatatgctatgcggcagtacttcctgaaagtgaagaaccagatgatcctgttcggcgaagaggacgtgcgcgtggccaatctgaagcccagaatcagcttcaacttcttcgtgacagcccctaagaacgtgtccgatatcatccccagaaccgaggtggaaaaggccatcagaatgagcagaagccggatcaacgacgccttccggctgaacgacaatagcctggaatttctgggcatccagcctacactgggccctccaaatcagcctcctgttagcggaggaggaggatctggaggaggaggatccgctagcacaaaaggccctagcgtctttccactcgccccatcctccaagagcacctctgggggcactgcggctttgggctgcttggtgaaggattacttcccagagcctgtcactgtcagctggaatagcggggctctgaccagtggagtgcacacctttcccgccgtgttacagagcagcggcctctatagcctgagcagtgtggtaactgtgccctcgagcagcttgggtacccagacctatatctgcaatgtcaatcataaacccagtaacacaaaggtagacaagaaagtcgaacccaaatcttgcgacaaaactcacacatgccctccttgccccgcccccgagctcctggggggaccttcggtttttctttttccacctaaaccgaaagacaccctgatgatcagcaggacaccagaagtgacatgtgtcgtcgtggacgtatcccatgaggacccggaggtgaagttcaactggtatgttgatggtgtggaagtgcataatgcaaaaactaagccacgggaggaacagtacaatagcacatacagagtagttagcgttctgactgtcctgcaccaagattggctgaacgggaaggaatacaagtgtaaggtgagcaacaaagccctgcccgctccaattgagaaaacaatttctaaggccaaaggacagccccgggagccacaggtgtatactctgccgcctagcagggaggaaatgaccaagaaccaggtgagcctgacctgtctggtgaaaggcttctaccccagcgatatcgcagttgagtgggagagcaatggccaacccgagaacaactacaagactacgccccccgtgcttgatagcgacggatcatttttcctgtactcaaaactgaccgtggacaaaagcagatggcagcagggaaacgttttcagttgctcagtgatgttcgaagcgctgcacaatcattatactcagaaaagcctgagcttgagcccgggaaaa